(19) United States
(12) Patent Application Publication
(10) Pub. No.: US 2026/0191927 A1
(43) Pub. Date: Jul. 9, 2026

(54) FORMULATION OF THERAPEUTIC AGENTS FOR IMPROVED PERMEATION

(71) Applicant: Vivtex Corporation, Boston, MA (US)

(72) Inventors: Thomas VON ERLACH, Mashpee, MA (US); James L. WRIGHT, Lexington, MA (US); Edgardo RIVERA-DELGADO, Cambridge, MA (US)

(21) Appl. No.: 19/368,009

(22) Filed: Oct. 24, 2025

Related U.S. Application Data
(63) Continuation of application No. PCT/US2024/026334, filed on Apr. 25, 2024.
(60) Provisional application No. 63/498,410, filed on Apr. 26, 2023.

(51) Int. Cl. A61K 38/14; A61K 31/57; A61K 31/7088; A61K 38/23; A61K 38/26; A61K 38/28

(52) U.S. Cl. CPC A61K 38/14; A61K 31/57; A61K 31/7088; A61K 38/23; A61K 38/26; A61K 38/28

(57) ABSTRACT

The present disclosure provides compositions and methods for enhanced delivery of a therapeutic agent to a subject, wherein the composition comprises the therapeutic agent, at least one bile acid or salt thereof, and optionally one or more co-excipients.

### CROSS-REFERENCE TO RELATED APPLICATIONS

This application claims the benefit of U.S. Provisional Application Ser. No. 63/498,410 filed Apr. 26, 2023, the entire contents of which are incorporated herein by reference.

### BACKGROUND

Routes of drug administration not requiring an injection (e.g., an intravenous, intramuscular, or subcutaneous injection) are often preferable due the convenience of drug administration, cost-effectiveness, and ease of large-scale manufacturing of non-injectable dosage forms (e.g., oral dosage forms). Additionally, in the case of at least oral formulations, compliance of patients is generally higher (see, e.g., Khan, K., et al. (2008) J. Behav. Med. 31 (3), 213-224). However, the effect of therapeutic agents administered via routes that require passage across a physiological barrier (e.g., an intestinal barrier) can be limited by their bioavailability (see, e.g., Martinez, M. N., et al. J. Clin. Pharmacol. 2002, 42, 620-643; Waring, M. J., et al. Nat. Rev. Drug Discov. 2015, 14, 475-486; Ward, K. W. In Reducing Drug Attrition; Topics in Medicinal Chemistry; Springer: Berlin, Germany, 2012). For example, oral bioavailability is dependent, at least in part, on physicochemical properties of the therapeutic agent such as solubility and intestinal permeability. Indeed, poor drug absorption in the small intestine presents a significant challenge in the development of oral drug formulations (see, e.g., Goldberg, et al (2003) Nat. Rev. Drug Discov. 2:289; Ensigna, et al (2012) Adv Drug Deliv Rev 64:557). Thus, there is a need for formulations that improve gastrointestinal (GI) permeation and bioavailability of therapeutic agents.

### SUMMARY OF THE DISCLOSURE

In some aspects, the present disclosure provides a composition comprising (i) a compound comprising a polypeptide having a molecular weight of about 1 kDa to about 250 kDa; and (ii) an amount of at least one bile acid or salt thereof at an effective concentration greater than a critical micelle concentration (CMC) of the bile salt.

In some aspects, the present disclosure provides a composition comprising (i) a compound comprising a nucleic acid; and (ii) an amount of at least one bile acid or salt thereof at an effective concentration greater than a CMC of the bile salt.

In some aspects, the disclosure provides a composition comprising (i) a class III or class IV compound according to the Biopharmaceutical Classification System (BCS); and (ii) an amount of at least one bile acid or salt thereof at an effective concentration greater than a CMC of the bile acid or salt thereof.

In some embodiments of any of the foregoing or related aspects, the composition comprises a pharmaceutically acceptable salt of the bile acid. In some embodiments, the bile acid or salt thereof comprises 3, 4, or 5 ring structures. In some embodiments, the bile acid or salt thereof comprises a steroid nucleus. In some embodiments, the steroid nucleus comprises one, two, or three hydroxy groups. In some embodiments, position 3 of the steroid nucleus comprises a hydroxy group. In some embodiments, position 7, position 12, or both of the steroid nucleus comprise a hydroxy group. In some embodiments, position 17 of the steroid nucleus comprises a carboxylate side chain. In some embodiments, position 17 of the steroid nucleus comprises an ester or amino side chain. In some embodiments, the amino side chain is selected from taurine and glycine. In some embodiments, the bile acid or salt thereof is selected from sodium taurocholate (STC), sodium glycocholate (GCA), sodium cholate (CHA), 3-[(3-Cholamidopropyl)dimethylammonio]-1-propanesulfonate (CHAPS), sodium taurodeoxycholate (TDCA), sodium glycodeoxycholate (GDCA), sodium deoxycholate (DCA), sodium taurochenodeoxycholate (TCDCA), sodium glycochenodeoxycholate (GCDCA), and sodium chenodeoxycholate (CDCA). In some embodiments, the bile acid or salt thereof has a CMC of less than about 30 mg/mL, about 20 mg/mL, about 15 mg/mL, or about 5 mg/mL. In some embodiments, the CMC is measured by a method selected from potentiometry, spectrophotometry, and light scattering.

In some aspects, the disclosure provides a composition comprising (i) a compound comprising a polypeptide having a molecular weight of about 1 kDa to about 250 kDa; and (ii) an amount of at least one bile acid or salt thereof at an effective concentration greater than a CMC of the bile salt, wherein the at least one bile acid or salt thereof is selected from glycocholic acid, taurocholic acid, and pharmaceutically acceptable salts thereof.

In some aspects, the disclosure provides a composition comprising (i) a compound comprising a nucleic acid; and (ii) an amount of at least one bile acid or salt thereof at an effective concentration greater than a critical micelle concentration (CMC) of the bile salt, wherein the at least one bile acid or salt thereof is selected from glycocholic acid, taurocholic acid, and pharmaceutically acceptable salts thereof.

In some aspects, the disclosure provides a composition comprising (i) a class III or class IV compound according to the Biopharmaceutical Classification System (BCS); and (ii) an amount of at least one bile acid or salt thereof at an effective concentration greater than a CMC of the bile salt, wherein the at least one bile acid or salt thereof is selected from glycocholic acid, taurocholic acid, and pharmaceutically acceptable salts thereof.

In some embodiments of any of the foregoing or related aspects, the composition comprises glycocholic acid or a pharmaceutically acceptable salt thereof. In some embodiments, the composition comprises taurocholic acid or a pharmaceutically acceptable salt thereof.

In some embodiments of any of the foregoing or related aspects, the amount of the at least one bile acid or salt thereof is effective to increase GI permeability of the polypeptide relative to GI permeability of the polypeptide alone. In some embodiments, the amount of the at least one bile acid or salt thereof is effective to increase intestinal permeation of the polypeptide relative to intestinal permeation of the polypeptide alone. In some embodiments, the polypeptide has a molecular weight greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the polypeptide has a molecular weight of about 1 kDa to about 5 kDa, about 2 kDa to about 5 kDa, about 3 kDa to about 5 kDa, about 1 kDa to about 10 kDa, about 2 kDa to about 10 kDa, about 5 kDa to about 10 kDa, about 5 kDa to about 20 kDa, about 5 kDa to about 50 kDa, about 10 kDa to about 50 kDa, about 10 kDa to about 100 kDa, about 10 kDa to about 150 kDa, about 50 kDa to about 150 kDa, about 100 kDa to about 150 kDa, about 50 kDa to about 200 kDa, about 100 kDa to about 200 kDa, about 100 kDa to about 250 kDa, about 150 kDa to about 250 kDa, or about 200 kDa to about 250 kDa. In some embodiments, the polypeptide is selected from an enzyme, an antibody or antigen binding fragment thereof, an antibacterial agent, a hormone, a growth factor, a chemokine, a cell signaling factor, and a cytokine. In some embodiments, the polypeptide is an enzyme. In some embodiments, the polypeptide is a hormone. In some embodiments, the polypeptide is an antibody or an antigen binding fragment thereof. In some embodiments, the polypeptide is a growth factor. In some embodiments, the polypeptide is a chemokine. In some embodiments, the polypeptide is a cytokine. In some embodiments, the polypeptide is a cell signaling factor. In some embodiments, the polypeptide is a recombinant protein. In some embodiments, the polypeptide is a therapeutic peptide.

In some embodiments of any of the foregoing or related aspects, the amount of the at least one bile acid or salt thereof is effective to increase GI permeability of the nucleic acid relative to GI permeability of the nucleic acid alone. In some embodiments, the amount of the at least one bile acid or salt thereof is effective to increase intestinal permeation of the nucleic acid relative to intestinal permeation of the nucleic acid alone. In some embodiments, the nucleic acid comprises ribonucleosides, deoxyribonucleosides, or a combination thereof. In some embodiments, the nucleic acid is selected from an immunostimulatory oligonucleotide, an mRNA, a plasmid DNA, and an RNA interference oligonucleotide. In some embodiments, the nucleic acid is an immunostimulatory oligonucleotide. In some embodiments, the nucleic acid is an mRNA. In some embodiments, the nucleic acid is a plasmid DNA. In some embodiments, the nucleic acid is an RNA interference oligonucleotide. In some embodiments, the RNA interference oligonucleotide is selected from an siRNA, a shRNA, a miRNA, and an antisense oligonucleotide. In some embodiments, the antisense oligonucleotide is a gapmer. In some embodiments, the nucleic acid comprises one or more modified nucleosides. In some embodiments, the modification is selected from a modified sugar moiety, a modified internucleoside linkage, a modified nucleobase, and a combination thereof. In some embodiments, the composition comprises a viral vector comprising the nucleic acid.

In some embodiments of any of the foregoing or related aspects, the amount of the at least one bile acid or salt thereof is effective to increase GI permeability of the class III or class IV compound relative to GI permeability of the compound alone. In some embodiments, the amount of the at least one bile acid or salt thereof is effective to increase intestinal permeation of the class III or class IV compound relative to intestinal permeation of the compound alone. In some embodiments, the composition comprises a class III compound according to the BCS. In some embodiments, the composition comprises a class IV compound according to the BCS. In some embodiments, the compound has a molecular weight greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the compound has a molecular weight of about 0.1 to about 1 kDa. In some embodiments, the compound is selected from a polypeptide, a nucleic acid, an oligosaccharide, a small molecule, a lipid, and a combination thereof.

In some embodiments of any of the foregoing or related aspects, the effective concentration of the at least one bile acid or salt thereof is about 2-fold to about 200-fold greater than the CMC. In some embodiments, the effective concentration of the at least one bile acid or salt thereof is at least about 3-fold, about 4-fold, about 5-fold, about 6-fold, about 7-fold, about 8-fold, about 9-fold, or about 10-fold greater than the CMC. In some embodiments, the effective concentration is about 15-fold, about 20-fold, about 25-fold, about 30-fold, about 35-fold, about 40-fold, about 45-fold, about 50-fold, about 60-fold, about 70-fold, about 80-fold, about 90-fold, about 100-fold, about 110-fold, about 120-fold, about 130-fold, about 140-fold, about 150-fold, about 160-fold, about 170-fold, about 180-fold, about 190-fold, or about 200-fold greater than the CMC. In some embodiments, the effective concentration of the at least one bile acid or salt thereof is about 32 mg/mL to 160 mg/mL. In some embodiments, the effective concentration of the at least one bile acid or salt thereof is at least about 35 mg/ml, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 90 mg/mL, about 95 mg/mL, or about 100 mg/mL. In some embodiments, the amount of the at least one bile acid or salt thereof is effective to increase GI permeability of the compound relative to GI permeability of the compound alone.

In some embodiments, the amount of the at least one bile acid or salt thereof is effective to increase intestinal permeability of the compound relative to intestinal permeability of the compound alone. In some embodiments, the composition comprises an effective amount of the compound. In some embodiments, the composition comprises one, two, three, or four bile acids or salts thereof.

In some embodiments of any of the foregoing or related aspects, the composition further comprises an amount of at least one co-excipient.

In some embodiments, the at least one co-excipient is selected from a chelator, a polymer, a dendrimer, a nanoparticle, a lipid, an alkyl acid, and a combination thereof. In some embodiments, the amount of the at least one co-excipient is effective to increase permeation of the compound relative to intestinal permeation of a control composition, optionally wherein the control composition is formulated without the at least one co-excipient. In some embodiments, the at least one co-excipient is a chelator. In some embodiments, the chelator is selected from sodium ethyleneglycol-bis(P-aminoethyl)-N,N,N′,N′-tetraacetate (EGTA), sodium ethylenedinitrilotetraacetate (EDTA), sodium glutamate (AAE), and a combination thereof. In some embodiments, the composition comprises an amount of the chelator at a concentration of about 5 mg/mL to about 100 mg/mL.

In some embodiments, the at least one co-excipient is a polymer. In some embodiments, the polymer is a homopolymer or a copolymer, optionally wherein the copolymer is selected from a block copolymer, a random copolymer, a graft copolymer, and an alternate copolymer. In some embodiments, the polymer comprises poly(methacrylate), poly(ethyl acrylate), poly(ethylene glycol), hyaluronic acid, polysaccharide, chitosan, arginine, poly(2-hydroxyethylmethacrylate), poly(N-isopropyl acrylamide), poly(ethylenimine), poly(N-(2-hydroxypropyl) methacrylamide), poly(glycolic acid), poly(lactic acid), poly(lactic-co-glycolic acid), poly(caprolactone), poly(orthoester), poly(anhydride), poly(amide), poly(ester amide), poly(phosphoester), poly(alky cyanoacrylate), or a combination thereof. In some embodiments, the polymer has a molecular weight of about 0.5 kDa to about 100 kDa. In some embodiments, the polymer is branched, optionally wherein the polymer comprises a star, H-shaped, pom-pom, or comb-shaped architecture. In some embodiments, the polymer is selected from Eudragit E PO (EPO); Eudragit RL 100 (RL100); Poly(ethylene glycol) 1 kDa (PEG-1 kDa); Branched Poly(ethyleneimine) 800 Da (PEI:b0.8); Kollidon SR (KOLSR); Poly(methacrylic acid) 5 kDa (PMA:5); Poly(aspartic acid) 2 kDa (POLD:2); Poly(2-ethyloxazoline) 25 kDa (POXZ:25); Poly(glutamic acid) 50 kDa (POLE: 50); Branched Poly(ethyleneimine)-co-Poly(ethylene glycol) 500 Da (bPEIPPEG:0.5); Vivacoat (VIVA). In some embodiments, the composition comprises an amount of the polymer at a concentration of about 5 mg/mL to about 150 mg/mL.

In some embodiments, the at least one co-excipient is a dendrimer. In some embodiments, the dendrimer comprises a poly(amidoamine), poly(propyleneimine), polyamide, polyether, polyester, or phosphorous-based architecture. In some embodiments, the dendrimer is a generation 1 (G1), G2, G3, G4, G5, G6, G7, G8, G9, G10, or G11 dendrimer. In some embodiments, the dendrimer is selected from carboxylate terminal poly(amidoamine) dendrimer generation 3.5 (DENDAC3.5) and amine terminal poly(amidoamine) dendrimer generation 4 (DENDAMV4). In some embodiments, the composition comprises an amount of the dendrimer at a concentration of about 0.1 mg/mL to about 50 mg/mL.

In some embodiments, the at least one co-excipient is a microscopic particle. In some embodiments, the microscopic particle is a microparticle or a nanoparticle. In some embodiments, the microscopic particle is selected from an inorganic particle, a carbon particle, a ceramic particle, a metal particle, a polymeric particle, a liposome, a lipid nanoparticle, and a combination thereof. In some embodiments, the microscopic particle comprises a metal oxide, a metal salt, a mineral colloid, an inorganic salt, and a combination thereof. In some embodiments, the microscopic particle is a carbon particle, optionally a fullerene or carbon nanotube. In some embodiments, the microscopic particle is selected from calcium phosphate tribasic microparticles (CaPO₄); calcium phosphate dibasic microparticles (CaHPO₄); 5 μm spherical hydroxyapatite microparticles (HAP:5U); 2.5 μm spherical hydroxyapatite microparticles (HAP:2.5U); zinc oxide microparticles (ZNO:r10n); hydroxyapatite rod shaped nanoparticles 40 nm diameter (HAP:r40n); carbon black nanoparticles 13 nm diameter (CB:13n). In some embodiments, the amount of the microscopic particle is at an effective concentration of about 0.1 mg/mL to about 100 mg/mL.

In some embodiments of any of the foregoing or related aspects, the amount (e.g., an amount in milligrams (mg)) of the at least one bile acid or salt thereof is substantially equivalent to the effective concentration (e.g., an effective concentration in units of weight per volume, e.g., mg/mL) multiplied by a dilution factor (e.g., a dilution factor in a volume, e.g., mL). In some embodiments, the effective concentration of the at least one bile acid or salt thereof is determined using a method to measure GI (e.g., intestinal) permeability described herein, e.g., a GI-ORIS screening assay. In some embodiments, the amount (e.g., an amount in milligrams (mg)) of the at least co-excipient is substantially equivalent to the effective concentration (e.g., an effective concentration in units of weight per volume, e.g., mg/mL) multiplied by a dilution factor (e.g., a dilution factor in a volume, e.g., mL). In some embodiments, the effective concentration of the at least one co-excipient is determined using a method to measure GI (e.g., intestinal) permeability described herein, e.g., a GI-ORIS screening assay. In some embodiments, the dilution factor is substantially equivalent to a liquid volume of the GI tract. In some embodiments, the dilution factor is substantially equivalent to a liquid volume of the GI tract in which the contents of the composition are released following administration. In some embodiments, the dilution factor is substantially equivalent to the liquid volume of the stomach, the liquid volume of the small intestine, or the sum of both. In some embodiments, the dilution factor (mL) is about 2 mL to about 30 mL. In some embodiments, the dilution factor (mL) is about 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL. In some embodiments, the composition is formulated for immediate release, wherein the dilution factor is less than about 30 mL, less than about 25 mL, less than about 20 mL, or less than about 15 mL. In some embodiments, the composition is formulated for immediate release, wherein the dilution factor is about 10 mL to about 20 mL, or about 10 mL, 11 mL, 12 mL, 13 mL, 14 mL, 15 mL, 16 mL, 17 mL, 18 mL, 19 mL, or 20 mL. In some embodiments, the composition is formulated for delayed release, wherein the dilution factor is less than about 15 mL, less than about 10 mL, or less than about 5 mL. In some embodiments, the composition is formulated for delayed release, wherein the dilution factor is about 2 mL to about 15 mL, or about 2 mL, 3 mL, 4 mL, 5 mL, 6 mL, 7 mL, 8 mL, 9 mL, 10 mL, 11 mL, 12 mL, 13 mL, 14 mL, 15 mL. In some embodiments, the amount of the at least one bile acid or salt thereof is about 60 mg to about 1,200 mg. In some embodiments, the amount of the at least co-excipient thereof is about 0.1 mg to about 600 mg.

In some aspects, the disclosure provides a dosage form comprising a composition described herein. In some embodiments, the amount of the at least one bile acid or salt thereof is sufficient to achieve the effective concentration at a site of absorption in the GI tract. In some embodiments, the dosage form is a solid dosage form. In some embodiments, the dosage form is a liquid dosage form. In some embodiments, the solid dosage form is formulated for immediate release. In some embodiments, the solid dosage form is formulated for delayed release. In some embodiments, the solid dosage form comprises an enteric coating. In some embodiments, the solid dosage form is formulated to release its contents in the stomach following oral administration. In some embodiments, the solid dosage form is formulated to release its contents in the small intestine following oral administration.

In some aspects, the disclosure provides a pharmaceutical composition comprising a composition described herein or a dosage form described herein, and a pharmaceutically acceptable carrier. In some embodiments, the pharmaceutical composition is formulated for enteral administration. In some embodiments, the pharmaceutical composition is formulated for rectal administration. In some embodiments, the pharmaceutical composition is formulated for gastric or intraduodenal administration. In some embodiments, the pharmaceutical composition is formulated for oral administration. In some embodiments, the pharmaceutical composition is formulated for sublingual or buccal administration. In some embodiments, the pharmaceutical composition is formulated for ocular or nasal administration. In some embodiments, the pharmaceutical composition is formulated for topical administration.

In some aspects, the disclosure provides a method of enhancing GI permeability of a compound, the method comprising administering a composition described herein, a dosage form described herein, or a pharmaceutical composition described herein to GI tissue. In some embodiments, the GI tissue comprises stomach tissue, intestinal tissue, rectal tissue, or a combination thereof. In some embodiments, the GI permeability of the compound is increased relative to GI permeability of a control composition. In some embodiments, the control composition comprises the compound alone. In some embodiments, GI permeability of the compound is increased by at least about 1.1-fold, about 1.2-fold, about 1.3-fold, about 1.4-fold, about 1.5-fold, about 2-fold, about 3-fold, about 4-fold, about 5-fold, about 6-fold, about 7-fold, about 8-fold, about 9-fold, or about 10-fold relative to GI permeability of the control composition. In some embodiments, the GI permeability of the compound is increased by about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 150-fold, about 200-fold, about 250-fold, or about 300-fold relative to GI permeability of the control composition.

In some aspects, the disclosure provides a method of enhancing intestinal permeability of a compound, the method comprising administering a composition described herein, a dosage form described herein, or a pharmaceutical composition described herein to the intestinal tissue. In some embodiments, the intestinal permeability of the compound is increased relative to intestinal permeability of a control composition. In some embodiments, the control composition comprises the compound alone. In some embodiments, intestinal permeability of the compound is increased by at least about 1.1-fold, about 1.2-fold, about 1.3-fold, about 1.4-fold, about 1.5-fold, about 2-fold, about 3-fold, about 4-fold, about 5-fold, about 6-fold, about 7-fold, about 8-fold, about 9-fold, or about 10-fold relative to intestinal permeability of the control composition.

In some embodiments, the intestinal permeability of the compound is increased by about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 150-fold, about 200-fold, about 250-fold, or about 300-fold relative to intestinal permeability of the control composition.

In some embodiments of any of the foregoing or related aspects, GI permeability (e.g., intestinal permeability) is measured in vivo or in vitro using a transwell permeability assay, a Ussing chamber assay, or a GI Tract-Organ Robotic Interface System (GI-ORIS)-based permeability assay.

In some aspects, the disclosure provides a method of increasing bioavailability of a compound in a subject, the method comprising administering a composition described herein, a dosage form described herein, or a pharmaceutical composition described herein to the GI tract of the subject, wherein the composition, the dosage form, or the pharmaceutical composition comprises an effective dose of the compound.

In some embodiments of any of the foregoing or related aspects, the administering is in vivo. In some embodiments, the administering is enteral. In some embodiments, the administering is rectal, intraduodenal, or oral. In some embodiments, the administering is sublingual or buccal.

In some aspects, the disclosure provides a method of increasing bioavailability of a compound in a subject, the method comprising orally administering a composition described herein, a dosage form described herein, or a pharmaceutical composition described herein to the subject, wherein the composition, the dosage form, or the pharmaceutical composition comprises an effective dose of the compound.

In some embodiments of any of the foregoing or related aspects, the bioavailability of the compound is increased relative to bioavailability of a control composition. In some embodiments, the control composition comprises the compound alone. In some embodiments, the plasma maximum peak of the compound is increased relative to the plasma maximum peak of a control composition. In some embodiments, the control composition comprises the compound alone.

In some aspects, the disclosure provides a method of preventing or treating a disease or disorder in a subject, comprising administering to the subject a composition described herein, a dosage form described herein, or a pharmaceutical composition described herein to the subject, wherein the composition, the dosage form, or the pharmaceutical composition comprises an effective dose of the compound. In some embodiments, the administration is enteral. In some embodiments, the administration is rectal. In some embodiments, the administration is intraduodenal. In some embodiments, the administration is oral. In some embodiments, the administration is sublingual or buccal. In some embodiments, the administration is topical. In some embodiments, the administration is dermal. In some embodiments, the administration is nasal. In some embodiments, the administration is ocular.

In some aspects, the disclosure provides a method of preventing or treating a disease or disorder in a subject, comprising orally administering to the subject a composition described herein, a dosage form described herein, or a pharmaceutical composition described herein to the subject, wherein the composition, the dosage form, or the pharmaceutical composition comprises an effective dose of the compound.

In some aspects, the disclosure provides use of a composition described herein, a dosage form described herein, or a pharmaceutical composition described herein for increasing bioavailability of a compound in a subject.

In some aspects, the disclosure provides use of a composition described herein, a dosage form described herein, or a pharmaceutical composition described herein in the manufacture of a medicament for increasing bioavailability of a compound in a subject.

In some aspects, the disclosure provides use of a composition described herein, a dosage form described herein, or a pharmaceutical composition described herein for preventing or treating a disease or disorder in a subject

In some aspects, the disclosure provides use of a composition described herein, a dosage form described herein, or a pharmaceutical composition described herein in the manufacture of a medicament for preventing or treating a disease or disorder in a subject

In some aspects, the disclosure provides a kit comprising a container comprising a composition described herein, a dosage form described herein, or a pharmaceutical composition described herein, and a package insert comprising instructions for administering the composition or pharmaceutical composition for increasing bioavailability of a compound in a subject from the GI tract.

In some aspects, the disclosure provides a kit comprising a container comprising a composition described herein, a dosage form described herein, or a pharmaceutical composition described herein, and a package insert comprising instructions for orally administering the composition or pharmaceutical composition for increasing bioavailability of a compound in a subject.

In some aspects, the disclosure provides a kit comprising a container comprising a composition described herein, a dosage form described herein, or a pharmaceutical composition described herein, and a package insert comprising instructions for administering the composition or pharmaceutical composition for preventing or treating a disease or disorder in a subject.

In some aspects, the disclosure provides a kit comprising a container comprising a composition described herein, a dosage form described herein, or a pharmaceutical composition described herein, and a package insert comprising instructions for orally administering the composition or pharmaceutical composition for preventing or treating a disease or disorder in a subject.

### BRIEF DESCRIPTION OF THE FIGURES

FIGS. 1A-1D provide bar graphs showing the enhancement in intestinal permeability measured in vitro using the GI Tract-Organ Robotic Interface System (GI-ORIS) for human calcitonin (FIG. 1A), human insulin (FIG. 1B), semaglutide (FIG. 1C), or vancomycin (FIG. 1D) formulated with a bile acid or a combination of bile acids identified in Table 3. Enhancement in peptide permeability is expressed as fold change increase in permeability relative to unformulated buffer control (bars represent the average of 6 replicates and error bars represent the standard deviation).

FIGS. 2A-2B respectively provide a beeswarm plot and bar plot quantifying permeability measured by GI-ORIS for vancomycin formulated with sodium glycocholate (GCA) at different concentrations (formulations corresponding to the formulation IDs in FIG. 2B are provided in Table 4). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability at the lowest concentration for which a maximal effect was observed (GCA at 128 mg/mL).

FIGS. 3A-3B respectively provide a beeswarm plot and bar plot quantifying permeability measured by GI-ORIS for vancomycin formulated with sodium taurocholate (STC) at different concentrations (formulations corresponding to the formulation IDs in FIG. 3B are provided in Table 5). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability at the lowest concentration for which a maximal effect was observed (STC at 128 mg/mL).

FIGS. 4A-4B respectively provide a beeswarm plot and bar plot quantifying permeability measured by GI-ORIS for vancomycin formulated with GCA and a chelator (formulations corresponding to the formulation IDs in FIG. 4B are provided in Table 6). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with GCA alone at 128 mg/mL).

FIGS. 5A-5B respectively provide a beeswarm plot and bar plot quantifying permeability measured by GI-ORIS for vancomycin formulated with STC and a chelator (formulations corresponding to the formulation IDs in FIG. 5B are provided in Table 7). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with STC alone at 128 mg/mL).

FIGS. 6A-6B respectively provide a beeswarm plot and bar plot quantifying permeability measured by GI-ORIS for vancomycin formulated with GCA and a polymer (formulations corresponding to the formulation IDs in FIG. 6B are provided in Table 8). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with GCA alone at 128 mg/mL).

FIGS. 7A-7B respectively provide a beeswarm plot and bar plot quantifying permeability measured by GI-ORIS for vancomycin formulated with STC and a polymer (formulations corresponding to the formulation IDs in FIG. 7B are provided in Table 9). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with STC alone at 128 mg/mL).

FIGS. 8A-8B respectively provide a beeswarm plot and bar plot quantifying permeability measured by GI-ORIS for vancomycin formulated with GCA and a dendrimer (formulations corresponding to the formulation IDs in FIG. 8B are provided in Table 10). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with GCA alone at 128 mg/mL).

FIGS. 9A-9B respectively provide a beeswarm plot and bar plot quantifying permeability measured by GI-ORIS for vancomycin formulated with GCA and a nanoparticle (formulations corresponding to the formulation IDs in FIG. 9B are provided in Table 11). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with GCA alone at 128 mg/mL).

FIGS. 10A-10B respectively provide a beeswarm plot and bar plot quantifying permeability measured by GI-ORIS for vancomycin formulated with STC and a nanoparticle (formulations corresponding to the formulation IDs in FIG. 10B are provided in Table 12). Enhancement in permeability for vancomycin is expressed as the average of the logarithm of inter-experiment scaled vancomycin permeability, with each circle or bar corresponding to an average of all experimental data for a given formulation over 1-5 experiments (n=6-30) (darker shading indicates increased vancomycin permeability and the dashed line indicates vancomycin permeability formulated with STC alone at 128 mg/mL).

FIG. 11 provides a two-dimensional heat map quantifying permeability measured by G1-ORIS for vancomycin formulated with bile salt alone (GCA or STC) or bile salt (GCA or STC) combined with a bioceramic nanoparticle (hydroxyapatite microparticles, zinc oxide microparticles), each at different concentrations. Values are average logarithm of inter-experiment scaled vancomycin permeability, aggregated over 2-4 experiments (n=12-24). Cells are shaded lighter with increasing vancomycin permeability.

FIG. 12A provides a two-dimensional heat map quantifying permeability measured by GI-ORIS for vancomycin formulated with bile salt alone (GCA or STC) or bile salt (GCA or STC) combined with a chelating agent (egtazic acid or glutamic acid), each at different concentrations. Values are average logarithm of inter-experiment scaled Vancomycin permeability, aggregated over 2-4 experiments (n=12-24). Cells are shaded lighter with increasing vancomycin permeability.

FIG. 12B provides a two-dimensional heat map quantifying permeability measured by GI-ORIS for human insulin formulated with bile salt alone (GCA or STC) or bile salt (GCA or STC) combined with a chelating agent (egtazic acid or glutamic acid), each at different concentrations. Values are average fold change enhancement in permeability relative to unformulated buffer control (n=6). Cells are shaded lighter with increasing insulin permeability.

FIG. 12C provides a two-dimensional heat map quantifying permeability measured by GI-ORIS for semaglutide formulated with bile salt alone (GCA or STC) or bile salt (GCA or STC) combined with a chelating agent (egtazic acid or glutamic acid), each at different concentrations. Values are average percent permeability of semaglutide (n=6). Cells are shaded lighter with increasing semaglutide permeability.

FIG. 12D provides a two-dimensional heat map quantifying permeability measured by GI-ORIS for human calcitonin formulated with bile salt alone (STC) or bile salt (STC) combined with a chelating agent (egtazic acid), each at multiple concentrations. Values are average percent permeability of calcitonin (n=6). Cells are shaded lighter with increasing calcitonin permeability.

FIG. 13 provides a bar graph quantifying permeability measured by GI-ORIS for vancomycin formulated with STC and a chelator (formulations corresponding to the formulation IDs are provided in Table 13). Values are average logarithm of inter-experiment scaled vancomycin permeability, aggregated over 1 experiment (n=6).

FIG. 14 provides a bar graph quantifying in vivo bioavailability of semaglutide following an intraduodenal injection in rats of a benchmark formulation of semaglutide with sodium caprate or a formulation of semaglutide with a bile acid (STC) and additional co-excipient (formulations corresponding to the formulation IDs are identified in Table 14). Control rats received an intravenous injection of the formulation. Bioavailability is shown as the fraction of plasma peptide concentration following intraduodenal administration compared to plasma peptide concentration following intravenous administration (% F) (** indicates a p value less than 0.01 compared to the benchmark formulation).

FIG. 15 provides a bar graph quantifying in vivo bioavailability of vancomycin following an intraduodenal injection in rats of unformulated vancomycin in buffer-control or vancomycin formulated with a bile acid (GCA or STC) and additional co-excipient (formulations corresponding to the formulation IDs are identified in Table 15). Control rats received an intravenous injection of the formulation. Bioavailability is shown as the fraction of plasma peptide concentration following intraduodenal administration compared to plasma peptide concentration following intravenous administration (% F) (* indicates a p value less than 0.05 in comparison to the unformulated vancomycin buffer control).

FIG. 16 provides a plot quantifying permeability measured by GI-ORIS for an antisense oligonucleotide formulated with a bile acid alone or bile acid and additional co-excipient (formulations corresponding to the formulation IDs are identified in Table 18). Values are the percent permeability for individual replicates in a single GI-ORIS screening experiment.

FIG. 17 provides a plot of oral bioavailability in dogs for a solid dosage formulation of vancomycin and intestinal permeability measured by GI-ORIS for a corresponding liquid formulation. A bivariate linear regression fit to the data indicated a highly significant correlation upon applying a dilution factor of 15 mL.

FIG. 18 provides a plot quantifying dose-normalized area under the curve (AUC) of semaglutide in plasma following oral administration to dogs of immediate release solid dosage forms containing semaglutide and the indicated excipients. Positive control animals received oral administration of Rybelsus (a commercially available oral semaglutide immediate release solid dosage form).

FIG. 19 provides a plot quantifying bioavailability of vancomycin following oral administration in dogs of immediate release (uncoated) or delayed release (enteric coated) solid dosage forms containing vancomycin and the indicated excipients. Comparison is made to bioavailability of an orally administered vancomycin solid dosage form without excipients (“non-formulated”). Bioavailability is shown as the fraction of vancomycin concentration following oral administration compared to plasma vancomycin concentration following intravenous administration.

### DETAILED DESCRIPTION

The present disclosure is based, at least in part, on the discovery of formulations for effective delivery of therapeutic agents to the gastrointestinal (GI) tract. As described herein, an automated high-throughput screening assay based on a GI Tract-Organ Robotic Interface System (GI-ORIS) was used to evaluate formulations for improving GI permeation of therapeutic agents, e.g., therapeutic agents characterized by poor intestinal permeation, wherein the formulations comprised at least one bile acid or salt thereof and optionally one or more co-excipients. GI-ORIS has several advantages for screening compositions for intestinal permeability of a therapeutic agent contained therein. First, GI-ORIS has been shown to have a higher predictive capacity for human oral drug absorption as compared to the traditional in vitro monolayer assays that use a tumor-derived cancer cell line from the colon or intestine (e.g., Caco-2 cell-based system) (see, e.g., von Erlach et al (2020) Nat Biomed Eng 4:544). Second, and without being bound by theory, the GI-ORIS cellular architecture remains substantially viable in the presence of elevated concentrations of a bile acid or salt thereof (e.g., concentrations exceeding the critical micellar concentration (CMC) of the bile acid or salt thereof). In contrast, traditional cell-based in vitro screening systems (e.g., Caco-2 cell-based system) are considered susceptible to cytotoxicity in the presence of such concentrations. As demonstrated herein, these features of GI-ORIS enabled the evaluation of compositions effective for improving GI (e.g., intestinal and/or stomach) permeation of a therapeutic agent, wherein the compositions comprised the therapeutic agent and at least one bile acid or salt thereof at a concentration greater than the CMC. It was also discovered that such compositions further comprising one or more co-excipients (e.g., a chelating agent) provided increased GI (e.g., intestinal and/or stomach) permeation. For example, as described herein, compositions of the disclosure were found to increase intestinal permeation by at least 2-fold compared to GI permeation of a control composition (e.g., a composition comprising the therapeutic agent without the at least one bile acid or salt thereof and the optional one or more co-excipients). Moreover, it was found that compositions of the disclosure characterized by GI-ORIS as having enhanced GI permeation were effective for increasing systemic bioavailability of the therapeutic agent following in vivo administration to the GI tract.

Accordingly, in some embodiments, the disclosure provides compositions and methods for improving delivery of therapeutic agents to the GI tract, including those that are characterized as having poor intestinal permeation. In some embodiments, the disclosure provides compositions and methods for improving delivery of therapeutic agents across a skin barrier or mucosal barrier. In some embodiments, the disclosure provides compositions and methods for improving delivery of therapeutic agents by routes of administration that do not require direct injection to a bodily tissue and/or the circulatory system (e.g., via routes of administration that do not require injection via parenteral routes, such as intramuscular, subcutaneous, or intravenous injection). In some embodiments, the disclosure provides compositions and methods for improving delivery of therapeutic agents by enteral administration (e.g., oral, duodenal, or rectal administration). In some embodiments, the disclosure provides compositions and methods for improving delivery of therapeutic agents by oral, buccal, or sublingual administration. In some embodiments, the disclosure provides compositions and methods for improving delivery of therapeutic agents by nasal or ocular administration.

In some embodiments, the disclosure provides a composition comprising a therapeutic agent, at least one bile acid or salt thereof, and optionally one or more co-excipients, wherein the composition is effective for increasing gastrointestinal (GI) permeability (e.g., intestinal permeability and/or stomach permeability) of the therapeutic agent following enteral administration (e.g., oral, duodenal, or rectal administration) to a subject. In some embodiments, the GI permeability of the therapeutic agent is increased compared to GI permeability of a control composition comprising the therapeutic agent, but lacking the at least one bile acid or salt thereof and the optional one or more co-excipients. In some embodiments, the intestinal permeability of the therapeutic agent is increased compared to intestinal permeability of the control composition. In some embodiments, the stomach permeability of the therapeutic agent is increased compared to stomach permeability of the control composition. In some embodiments, the administration is oral. In some embodiments, the administration is duodenal (e.g., via a feeding tube). In some embodiments, the administration is rectal.

In some embodiments, the disclosure provides a composition comprising a therapeutic agent, at least one bile acid or salt thereof, and optionally one or more co-excipients, wherein the composition is effective for increasing permeability across a skin barrier of the therapeutic agent following dermal or topical administration to a subject. In some embodiments, the permeability of the therapeutic agent across the skin barrier is increased compared to permeability of a control composition comprising the therapeutic agent, but lacking the at least one bile acid or salt thereof and the optional one or more co-excipients.

In some embodiments, the disclosure provides a composition comprising a therapeutic agent, at least one bile acid or salt thereof, and optionally one or more co-excipients, wherein the composition is effective for increasing permeability across a mucosal barrier of the therapeutic agent following nasal or ocular administration to a subject. In some embodiments, the administration is nasal (e.g., via inhalation). In some embodiments, the administration is ocular. In some embodiments, the disclosure provides a composition comprising a therapeutic agent, at least one bile acid or salt thereof, and optionally one or more co-excipients, wherein the composition is effective for increasing permeability across a mucosal barrier of the therapeutic agent following buccal or sublingual administration to a subject. In some embodiments, the permeability of the therapeutic agent across the mucosal barrier is increased compared to permeability of a control composition comprising the therapeutic agent, but lacking the at least one bile acid or salt thereof and the optional one or more co-excipients.

In some embodiments, the composition is formulated as a solid dosage form or a liquid dosage form. In some embodiments, the composition is formulated as a solid dosage form or a liquid dosage form for enteral administration (e.g., oral, duodenal, or rectal administration). In some embodiments, the solid dosage form is an immediate release formulation. In some embodiments, the solid dosage form is a delayed release formulation. In some embodiments, the solid dosage form comprises an enteric coating for delayed release. Methods for formulating a dosage form for enteral administration are known in the art and further described herein. In some embodiments, the dosage form comprises an amount of the at least one bile acid or salt thereof sufficient to achieve an effective concentration of the at least one bile acid or salt thereof at the site of absorption (e.g., the GI tract). For example, in some embodiments, the amount is sufficient to achieve an effective concentration at the site of absorption despite dilution that occurs in vivo between administration and transport to the site of absorption. In some embodiments, the effective concentration is greater than the critical micelle concentration (CMC) of the at least one bile acid or salt thereof. In some embodiments, the effective concentration of the at least one bile acid or salt thereof is determined using a method to measure GI (e.g., intestinal) permeability described herein, e.g., a GI-ORIS screening assay.

In some embodiments, the disclosure provides methods to increase GI (e.g., intestinal and/or stomach) permeability of a therapeutic agent following enteral administration (e.g., oral, duodenal, or rectal administration) to a subject, the method comprising formulating a dosage form (e.g., a solid dosage form or a liquid dosage form) comprising a composition of the disclosure, wherein the composition comprises one or more doses of the therapeutic agent, an amount of at least one bile acid or salt thereof sufficient to achieve an effective concentration of the at least one bile acid or salt thereof at the site of absorption (e.g., wherein the effective concentration is determined using a method described herein to measure GI permeability such as GI-ORIS), and optionally an amount of one or more co-excipients, and administering the dosage form enterally (e.g., via oral, duodenal, or rectal administration) to the subject, wherein GI (e.g., intestinal and/or stomach) permeability of the therapeutic agent is increased compared to GI permeability of a dosage form comprising a control composition (e.g., a composition lacking the at least one bile acid or salt thereof and the optional one or more co-excipients). In some embodiments, increased GI (e.g., intestinal and/or stomach) permeability of the therapeutic agent is effective for increasing its bioavailability following enteral administration. Without being bound by theory, one or more benefits of increasing the bioavailability of the therapeutic agent are that (i) a reduced dose is required to achieve a minimum effective concentration threshold, thereby mitigating or reducing the risk of undesirable effects associated with higher doses of the therapeutic agent (e.g., toxicity, adverse symptoms); (ii) avoiding a requirement to administer the therapeutic agent by needle injection, thereby improving patient compliance and adherence; and/or (iii) avoiding the need for hospitalization, such as in the case of intravenous injection.

### Compositions of the Disclosure for Enhanced Permeability

The present disclosure provides compositions comprising a therapeutic agent, at least one bile acid or salt thereof, and optionally one or more co-excipient. In some embodiments, the disclosure provides compositions comprising a therapeutic agent and at least one bile acid or salt thereof. In some embodiments, the disclosure provides compositions comprising a therapeutic agent, at least one bile acid or salt thereof, and one or more co-excipients.

In some embodiments, the composition comprises one or more doses of the therapeutic agent. In some embodiments, the composition comprises one bile acid or salt thereof. In some embodiments, the composition comprises more than one bile acid or salt thereof. In some embodiments, the composition comprises two bile acids or salts thereof. In some embodiments, the composition comprises three bile acids or salts thereof. In some embodiments, the composition comprises four bile acids or salts thereof. In some embodiments, the composition comprises five bile acids or salts thereof.

In some embodiments, the at least one bile acid or salt thereof, and optionally the one or more co-excipients, enhances the GI (e.g., intestinal and/or stomach) permeation of the therapeutic agent in a subject (e.g., a human subject). In some embodiments, the at least one bile acid or salt thereof, and optionally the one or more co-excipients, enhances the intestinal permeation of the therapeutic agent in a subject (e.g., a human subject). In some embodiments, the at least one bile acid or salt thereof enhances the GI (e.g., intestinal and/or stomach) permeation of the therapeutic agent in a subject (e.g., a human subject). In some embodiments, the at least one bile acid or salt thereof enhances the intestinal permeation of the therapeutic agent in a subject (e.g., a human subject). In some embodiments, the at least one bile acid or salt thereof, and the one or more co-excipients, enhances the GI (e.g., intestinal and/or stomach) permeation of the therapeutic agent in a subject (e.g., a human subject). In some embodiments, the at least one bile acid or salt thereof, and the one or more co-excipients, enhances the intestinal permeation of the therapeutic agent in a subject (e.g., a human subject). In some embodiments, the GI (e.g., intestinal and/or stomach) permeation is enhanced compared to the GI (e.g., intestinal and/or stomach) permeation of a control composition, wherein the control composition lacks the at least one bile acid or salt thereof and the optional one or more co-excipients. In some embodiments, the intestinal permeation is enhanced compared to the intestinal permeation of a control composition, wherein the control composition lacks the at least one bile acid or salt thereof and the optional one or more co-excipients.

### Therapeutic Agents

In some embodiments, a therapeutic agent of a composition described herein is characterized as having poor permeability across one or more physiological barriers. In some embodiments, the therapeutic agent is a macromolecule (e.g., a therapeutic agent having a molecular weight greater than about 1 kDa) with poor permeability across one or more physiological barriers. In some embodiments, the therapeutic agent is a polypeptide (e.g., a polypeptide having a molecular weight of about 1 kDa to about 250 kDa) with poor permeability across one or more physiological barriers. In some embodiments, the therapeutic agent is a nucleic acid with poor permeability across one or more physiological barriers. In some embodiments, the therapeutic agent is a compound having a molecular weight less than 1 kDa with poor permeability across one or more physiological barriers. In some embodiments, the physiological barrier comprises a gastrointestinal barrier (e.g., a stomach and/or intestinal barrier). In some embodiments, the physiological barrier comprises skin. In some embodiments, the physiological barrier comprises a mucosal barrier, a cellular lining (e.g., an epithelial cell lining), and/or a chemical lining (e.g., a lining comprising digestive secretions, antimicrobial peptides, and/or cellular secretions). In some embodiments, a therapeutic agent of a composition described herein is characterized as having poor GI (e.g., stomach and/or intestinal) permeability. In some embodiments, the therapeutic agent is characterized as having poor GI permeability due to size (e.g., a molecular weight greater than 1 kDa), charge, hydrophobicity, or a combination thereof. In some embodiments, the therapeutic agent is characterized as having poor GI permeability according to the Biopharmaceutical Classification System (BCS) system.

In some embodiments, the therapeutic agent is a macromolecule (e.g., a therapeutic agent having a molecular weight greater than about 1 kDa) with poor GI (e.g., stomach and/or intestinal) permeability. In some embodiments, the therapeutic agent is a polypeptide (e.g., a polypeptide having a molecular weight of about 1 kDa to about 250 kDa) with poor GI (e.g., stomach and/or intestinal) permeability. In some embodiments, the therapeutic agent is a nucleic acid with poor GI (e.g., stomach and/or intestinal) permeability. In some embodiments, the therapeutic agent is a small molecule (e.g., a therapeutic agent having a molecular weight of less than about 1 kDa, e.g., a BSC class III or class IV compound) with poor GI (e.g., stomach and/or intestinal) permeability.

As used herein, the term “therapeutic agent” includes any compound having a therapeutic effect, including conventional drugs and analogs thereof, appropriate for administration to a subject (e.g., a human subject). In some embodiments, the therapeutic agent is a compound having an in vivo pharmacological activity, e.g., to mediate diagnosis, cure, mitigation, treatment, or prevention of a disease or disorder in a subject or to affect the structure or function of the body in the subject. In some embodiments, the therapeutic agent comprises a compound poorly absorbed via the enteral (e.g., oral, duodenal, or rectal) route including hydrophilic or macromolecular drugs, such as polypeptides, nucleic acids, oligosaccharides, polysaccharides, lipids, or hormones. In some embodiments, the therapeutic agent comprises a compound with poor permeation across the lining of the intestine and/or stomach. In some embodiments, the therapeutic agent comprises a compound with poor permeation across the skin. In some embodiments, the therapeutic agent comprises a compound with poor permeation across a mucosal barrier.

### Polypepides

In some embodiments, the therapeutic agent comprises a polypeptide. In some embodiments, the therapeutic agent is a polypeptide. In some embodiments, the polypeptide has a molecular weight of greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the polypeptide has a molecular weight of at least about 1 kDa and up to about 300 kDa. In some embodiments, the polypeptide has a molecular weight of at least about 10 kDa and up to about 300 kDa. In some embodiments, the polypeptide has a molecular weight of at least about 50 kDa and up to about 300 kDa. In some embodiments, the polypeptide has a molecular weight of at least about 100 kDa and up to about 300 kDa. In some embodiments, the polypeptide has a molecular weight of at least about 1 kDa and up to about 200 kDa. In some embodiments, the polypeptide has a molecular weight of at least about 10 kDa and up to about 200 kDa. In some embodiments, the polypeptide has a molecular weight of at least about 50 kDa and up to about 200 kDa. In some embodiments, the polypeptide has a molecular weight of at least about 100 kDa and up to about 200 kDa. In some embodiments, the polypeptide has a molecular weight of at least about 1 kDa and up to about 100 kDa. In some embodiments, the polypeptide has a molecular weight of at least about 10 kDa and up to about 100 kDa. In some embodiments, the polypeptide has a molecular weight of at least about 50 kDa and up to about 100 kDa. In some embodiments, the polypeptide has a molecular weight of greater than about 0.1 kDa and up to about 1 kDa.

In some embodiments, the polypeptide is naturally-occurring. In some embodiments, the polypeptide is synthetic. In some embodiments, the polypeptide comprises an engineered variant of a protein (e.g., a recombinant protein) or fragment thereof.

In some embodiments, the polypeptide comprises an enzyme. In some embodiments, the polypeptide comprises an antibody or antigen binding fragment thereof. In some embodiments, the polypeptide comprises a hormone. In some embodiments, the polypeptide comprises an immunoglobulin. In some embodiments, the polypeptide comprises a growth factor. In some embodiments, the polypeptide comprises a cell signaling ligand. In some embodiments, the polypeptide comprises a cytokine. In some embodiments, the polypeptide comprises a chemokine. In some embodiments, the polypeptide comprises an antibacterial agent.

In some embodiments, the polypeptide comprises one or more modifications. In some embodiments, the one or more modifications are selected from any post-translational modification known in the art. Non-limiting examples of post-translational modifications include glycosylation, acylation, prenylation, lipoylation, phosphorylation, ubiquitinylation, S-nitrosylation, methylation, N-acetylation, lipidation, S-glutationylation, succinylation, sulfation, glycation, carbamylation, carbonylation, biotinylation, oxidation, pegylation, sumoylation, racemization, iodination, hydroxylation, malonylation, propionylation, butyrylation, carboxylation.

### Nucleic Acids

In some embodiments, the therapeutic agent comprises a nucleic acid. In some embodiments, the therapeutic agent is a nucleic acid. In some embodiments, the nucleic acid has a molecular weight of greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the nucleic acid has a molecular weight of about 0.1 to about 1 kDa.

In some embodiments, the nucleic acid comprises DNA. In some embodiments, the nucleic acid comprises plasmid DNA.

In some embodiments, the nucleic acid comprises RNA. In some embodiments, the RNA is a single-strand. In some embodiments, the single-strand comprises a duplex. In some embodiments, the single-strand comprises an RNA secondary structure (e.g., a hairpin loop, a stem-loop). In some embodiments, the RNA is a double-strand RNA. In some embodiments, the double-strand comprises a duplex and one or more overhangs. In some embodiments, the double-strand comprises a duplex and one or more bulges.

In some embodiments, the RNA comprises a small hairpin RNA (shRNA) molecule. In some embodiments, the RNA comprises a small interfering RNA (siRNA) molecule. molecule. In some embodiments, the RNA comprises a microRNA (miRNA) molecule. In some embodiments, the RNA comprises a messenger RNA (mRNA) molecule. In some embodiments, the RNA comprises a transfer RNA (tRNA) molecule. In some embodiments, the RNA comprises a small nuclear RNA (snRNA) molecule. In some embodiments, the RNA comprises a non-coding RNA (ncRNA) molecule.

In some embodiments, the nucleic acid comprises an oligonucleotide. As used herein, the term “oligonucleotide” includes RNA agents and DNA agents, as well as chimeric oligonucleotides that comprise both RNA and DNA elements (e.g., gapmers). Moreover, the term “oligonucleotide” includes compounds comprising naturally-occurring nucleotides, non-naturally-occurring nucleotides (e.g., nucleotide analogues) or a combination of naturally-occurring and non-naturally-occurring nucleotides. In one embodiment, the oligonucleotide is an RNA agent (i.e., an oligonucleotide whose sugar-phosphate backbone comprises ribose, or a chemical analogue thereof). In one embodiment, the oligonucleotide is a DNA agent (i.e., an oligonucleotide whose sugar-phosphate backbone comprises deoxyribose, or a chemical analogue thereof). In one embodiment, the oligonucleotide is a modified RNA agent, a non-limiting example of which is a locked nucleic acid (LNA)-containing RNA oligonucleotide. In some embodiments, the RNA agent comprises a single-stranded RNA, double-stranded RNA (dsRNA) or a molecule that is a partially double-stranded RNA, i.e., has a portion that is double-stranded and a portion that is single-stranded. In some embodiments, the RNA molecule is a circular RNA molecule or a linear RNA molecule. Such oligonucleotides are well established in the art.

Non-limiting examples of RNA agents include messenger RNAs (mRNAs) (e.g., encoding a protein of interest), modified mRNAs (mmRNAs) that include at least one chemical modification as compared to naturally-occuring RNA, mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA) and locked nucleic acids (LNAs). Such RNA agents are well established in the art.

In some embodiments, the DNA agent comprises a double-stranded DNA, single-stranded DNA (ssDNA), or a molecule that is a partially double-stranded DNA, i.e., has a portion that is double-stranded and a portion that is single-stranded. In some embodiments, the DNA molecule is triple-stranded or is partially triple-stranded, i.e., has a portion that is triple stranded and a portion that is double stranded. In some embodiments, the DNA molecule is a circular DNA molecule or a linear DNA molecule. Such oligonucleotides are well established in the art.

In one embodiment, the oligonucleotide is an antisense oligonucleotide, e.g., an antisense RNA. In some embodiments, the antisense RNAs (asRNAs), also referred to in the art as antisense transcripts, is a naturally-occurring or synthetically produced single-stranded RNA molecule complementary to a protein-coding messenger RNA (mRNA) with which it hybridizes and thereby blocks the translation of the mRNA into a protein. Antisense transcripts are classified into short (less than 200 nucleotides) and long (greater than 200 nucleotides) non-coding RNAs (ncRNAs). The primary natural function of asRNAs is in regulating gene expression and synthetic versions have been used widely as research tools for gene knockdown and for therapeutic applications. Antisense RNAs and their functions have been described in the art (see e.g., Weiss et al. (1999) Cell. Molec. Life Sci. 55:334-358; Wahlstedt (2013) Nat. Rev. Drug Disc. 12:433-446; Pelechano and Steinmetz (2013) Nat. Rev. Genet. 14:880-893). Accordingly, in some embodiments, a composition of the disclosure comprises an agent for antisense therapy. In one embodiment, the agent for antisense therapy is an RNA agent or chimeric oligonucleotide (e.g., gapmer) comprising at least one modification as compared to naturally-occurring ribonucleic acids, such as at least one chemical analogue of a naturally-occurring ribonucleic acid. In some embodiments, the modification of the RNA agent, as compared to naturally-occurring ribonucleic acids, comprises incorporation of at least one locked nucleic acid.

In some embodiments, the oligonucleotide comprises one or more locked nucleic acids. Locked nucleic acids, also referred to as inaccessible RNA, are modified RNA nucleotide molecules in which the ribose moiety of the LNA is modified with an extra bridge connecting the 2′ oxygen and the 4′ carbon. This bridge “locks” the ribose in the 3′-endo (North) conformation. LNA nucleotides can be mixed with DNA or RNA residues in an oligonucleotide whenever desired and hybridize with DNA or RNA according to Watson-Crick base-pairing rules. The locked ribose conformation enhances base stacking and backbone pre-organization. This significantly increases the hybridization properties (e.g., melting temperature) of oligonucleotides containing LNA nucleotides. LNA molecules, and their properties, have been described in the art (see e.g., Obika et al. (1997) Tetrahedron Lett. 38:8735-8738; Koshkin et al. (1998) Tetrahedron 54:3607-3630; Elmen et al. (2005) Nucl. Acids Res. 33:439-447).

In some embodiments, the antisense RNA is a gapmer. Gapmers are chimeric antisense oligonucleotides that contain a central block of deoxynucleotide monomers sufficiently long to induce RNAase H cleavage. Such gapmers are well established in the art. In some embodiments, the gapmer is a locked nucleic acid (LNA)-containing gapmer. The use of LNA-containing gapmer antisense oligonucleotides for antisense therapy is well established in the art (see e.g., Wahlestedt et al. (2000) Proc. Natl. Acad. Sci. USA 97:5633-5638; Kurreck et al. (2002) Nucl. Acids Res. 30:1911-1918; Fluiter et al. (2009) Mol. Biosyst. 5:838-843; Pendergraff et al. (2017) Mol. Therap. Nucl. Acids 8:158-168).

### BCS

The Biopharmaceutical Classification System (BCS) categorizes therapeutic agents (e.g., oral dosage forms of therapeutic agents) into different classes (BCS I-IV) based upon aqueous solubility and intestinal permeability, parameters that determine the fraction of the therapeutic agent absorbed following oral intake (see, e.g., Amidon, G. L. Pharm. Res. 12, 413-420 (1995); Benet, L. Z., AAPS J. 13, 519-547 (2011); Dahan, A., et al. Mol. Pharm. 9, 1847-1851 (2012); Lobenberg, R. et al. Eur. J. Pharm. Biopharm. Off. J. 50, 3-12 (2000)).

Characteristics of therapeutic agents (e.g., oral dosage forms of therapeutic agents) under BCS are known in the art (see, e.g., Khan G M. The Science (2001) 1:350-54; Lobenberg R, et al. Eur J Pharm Biopharm (2000) 50:3-12; Draft Guidance for Industry, “Waiver of in vivo Bioavailability and Bioequivalence Studies for Immediate Release Solid Oral Dosage Forms containing certain Active Moieties/Active Ingredients based on a Biopharmaceutic Classification System” CDER/FDA (2017)). A class I therapeutic agent according to BCS is characterized by high aqueous solubility and high intestinal permeability. A class II therapeutic agent according to BCS is characterized by low aqueous solubility and high intestinal permeability. A class III therapeutic agent according to BCS is characterized by high aqueous solubility and low intestinal permeability. A class IV therapeutic agent according to BCS is characterized by low aqueous solubility and low intestinal permeability.

The BCS comprises a class boundary to define aqueous solubility of a therapeutic agent. Aqueous solubility refers to the process by which a compound enters aqueous solution via mass transfer from a solid surface into the liquid phase (see Sharma, J. L. Dictionary of Chemistry. New Delhi India: CBS Publishers and Distributors; 1997. p. 558-60.). The degree of solubility is the concentration of the solute in a saturated solution at a given temperature. The rate of dissolution is the amount of solid substance that goes into solution per unit time under standard conditions of temperature, pH, solvent composition, and constant solid surface area. Under the BCS, a therapeutic agent has “high aqueous solubility” when the highest dose strength is soluble (e.g., at least 90% or more of the dose is soluble) in about 250 mL or less of aqueous media within the a pH range of about 1 to about 7.5 at about 37° C. (e.g., ±1° C., ±2° C., :3° C., ±4° C., ±5° C., ±6° C., ±7° C., ±8° C., ±9° C., or ±10° C.) (see, e.g., Draft Guidance for Industry, “Waiver of in vivo Bioavailability and Bioequivalence Studies for Immediate Release Solid Oral Dosage Forms containing certain Active Moieties/Active Ingredients based on a Biopharmaceutic Classification System”. CDER/FDA (2017)). The volume estimate of 250 mL for (i) is derived from typical bioequivalence study protocols that prescribe administration of a drug product to fasting human volunteers with a glass (approximately 8 ounces) of water. In some embodiments, the evaluation is performed by exposing the highest dose strength of the therapeutic agent to about 250 mL of an aqueous medium (e.g., water or aqueous buffer) and an assay to measure solubility is performed after equilibrium is established. The pH solubility profile of the drug is determined at about 37° C. (e.g., ±1° C., ±2° C., ±3° C., ±4° C., ±5° C., ±6° C., ±7° C., ±8° C., ±9° C., or ±10° C.) in the aqueous medium, with pH in the range of about 1 to about 7.5. In some embodiments, the aqueous medium is one described in the art (see, e.g., British Pharmacopoeia Electronic version: Appendix I D. Buffer Solutions. London: The Stationary Office; 2003.). Methods to measure aqueous solubility are known in the art and include the shake-flask or titration method or analysis by a validated stability-indicating assay. In some embodiments, a solubility indicating assay is selected that distinguishes the therapeutic agent from its degradation products.

The BCS comprises a class boundary to define intestinal permeability of a therapeutic agent. As further described herein, the GI tract is characterized by an intestinal barrier that prevents loss of water and electrolytes and entry of antigens or microorganisms into systemic circulation, while allowing exchange of compounds between the external environment and the host and absorption of nutrients from the diet. The intestinal wall comprises four layers: the mucosa, the submucosa, the muscularis, and the serosa. The outermost layer of the GI tract is the serosa, which is a smooth membrane comprising a thin layer of cells that secrete serous fluid and a thin layer of connective tissue. The next layer is the muscularis, which comprises two muscle layers that contribute to gut movement (peristalsis). The next layer is the submucosa which comprises a layer of dense irregular connective tissue or loose connective tissue that supports the mucosa and joints it to the underlying smooth muscle. The innermost layer that faces the lumen of the GI tract is the mucosa. The mucosa comprises the physical barrier of the GI tract (e.g., including the vascular endothelium, the epithelial cell lining, and the mucus layer) and a layer of secreted digestive substances, immune molecules, and cell products (e.g., inflammatory mediators, antimicrobial peptides, and cytokines). As used herein, the term “intestinal permeability” refers to the capacity of a compound to pass from the lumen of the GI tract to the submucosa, e.g., by entering or crossing the epithelium or the mucus layer respectively. According to the BCS, a therapeutic agent is considered to have “high intestinal permeability” if the systemic bioavailability is determined to be at least about 85% of an administered dose, e.g., based on a mass balance determination as described herein or in comparison to an intravenous reference dose.

Methods to measure intestinal permeability are known in the art. In some embodiments, permeability is measured using a mass balance study. For example, a radiolabeled drug substance is administered to one or more subjects (e.g., one or more human subjects), and the extent of absorption of the drug substance into systemic circulation is measured (e.g., by detection of the quantity of the radiolabeled drug present in a tissue sample such as blood or urine). In some embodiments, permeability is measured based on the extent of absorption (e.g., fraction of dose absorbed) of a therapeutic agent in a subject (e.g., a human subject). Methods to measure the extent of absorption are known in the art and include absolute bioavailability studies and intestinal perfusion studies (e.g., in vivo intestinal perfusion studies in humans, in vivo or in situ perfusion studies in animals). In some embodiments, permeability is measured in vitro using excised human or animal intestinal tissue or use of epithelial cell monolayers. Suitable methods are described in the Examples section herein. For example, in some embodiments, permeability is measured using an in vitro tissue explant according to GI-ORIS, as described in US Patent Pub. No. 2019/0064153, which is herein incorporated by reference. In another example, the Caco2 cell lines is derived from human colon carcinoma and used for permeability determination. Initial screening can also be carried out using parallel artificial membrane permeability analysis (PAMPA), which is carried out on microplates. It measures the permeation of compounds through a phospholipid-coated filter medium that mimics intestinal cell structures. In determining the permeability of a drug, reference standards of known permeability can be employed in the absorption assay.

In some embodiment, the therapeutic agent is characterized as an immediate release (IR) drug according to the BCS. In some embodiments, the IR drug is characterized by having rapid or very rapid dissolution. According to BCS, a therapeutic agent is considered to have “rapid dissolution” when an average of 85% or more of the labeled amount of drug substance dissolves within 30 minutes, using United States Pharmacopeia (USP) Apparatus 1 at 100 rpm or Apparatus 2 at 50 rpm in a volume of 500 mL or less of an aqueous dissolution media and “very rapid dissolution” when an average of 85% or more of the labeled amount of drug substance dissolves within 15 minutes under foregoing conditions. In some embodiments, the aqueous dissolution media is 0.1 N HCl. In some embodiments, the aqueous dissolution media is simulated gastric fluid (SGF) without enzymes. In some embodiments, the aqueous dissolution media is a pH 4.5 buffer. In some embodiments, the aqueous dissolution media is a pH 6.8 buffer. In some embodiments, the aqueous dissolution media is simulated intestinal fluid (SIF) without enzymes. In some embodiments, the aqueous dissolution media comprises SGF and surfactants. In some embodiments, the aqueous dissolution media comprises milk with 3.5% fat. In some embodiments, the aqueous dissolution media comprises low volume SIF. In some embodiments, the aqueous dissolution media comprises high volume SIF.

In some embodiments, the therapeutic agent is characterized as class III according to BCS. In some embodiments, the class III therapeutic agent has a molecular weight of greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the class III therapeutic agent has a molecular weight of about 0.1 to about 1 kDa. In some embodiments, the class III therapeutic agent comprises a polypeptide, a nucleic acid, an oligosaccharide, a small molecule, a lipid, or a combination thereof. In some embodiments, a Class III therapeutic agent is a biologic characterized by good water solubility and poor GI permeability, such as proteins, peptides, polysaccharides, nucleic acids, nucleic acid oligomers and viruses. Examples of Class III therapeutic agents include, but are not limited to, acyclovir, neomycin B, captopril, atenolol, valproic acid, stavudine, salbutamol, methotrexate, lamivudine, ergometrine, ciprogloxacin, amiloride and caspofungin.

In some embodiments, the therapeutic agent is characterized as class IV according to BCS. In some embodiments, the class IV therapeutic agent has a molecular weight of greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the class IV therapeutic agent has a molecular weight of about 0.1 to about 1 kDa. In some embodiments, the class IV therapeutic agent comprises a polypeptide, a nucleic acid, an oligosaccharide, a small molecule, a lipid, or a combination thereof. In some embodiments, a Class IV therapeutic agent is a lipophilic compound with poor GI permeability. Examples of Class IV drugs include, but are not limited to, nalidixic acid, clorothiazide, tobramycin, cyclosporin, allopurinol, acetazolamide, doxycyclin, dapsone, sulfamethoxazole, tacrolimus, paclitaxel, doxorubicin, blymicine, and griseofulvin.

Additional non-limiting examples of BCS Class III and Class IV therapeutic agents include abacavir sulfate, acetylsalicylic acid, amoxicillin, atropine sulfate, azathioprine, benznidazole, chloramphenicol, cimetidine, codein phosphate, colchicine, cyclophosphamide, dapsone, dexamethasone, didanosine, diethylcarbamazine citrate, digoxin, ethambutol hydrochloride, ethosuximide, fluconazole, folic acid, furosemide, griseofulvin, hydralazine hydrochloride, hydrochlorothiazide, isoniazid, methyldopa, methoclopramide hydrochloride, methronidazole, nicotinamide, nifurtimox, nitrofurantoin, nystatin, paracetamol, penicillamine, penicillin V potassium, phenobarbital, primaquine phosphate, propylthiouracil, pyrazinamide, pyridostigmine bromide, pyridoxine hydrochloride, pyrimethamine, sulfate, sulfadiazine, theophylline, trimethoprim, and zidovudine.

In some embodiments, the therapeutic agent (e.g., BCS Class III or IV therapeutic agent) comprises a polypeptide, a nucleic acid, an oligosaccharide, a small molecule, a lipid, and a combination thereof (e.g., a complex or conjugate). In some embodiments, the therapeutic agent comprises a complex or conjugate of a protein and nucleic acid. In some embodiments, the therapeutic agent comprises a complex or conjugate of a protein and oligosaccharide. In some embodiments, the therapeutic agent comprises a complex or conjugate of a protein and small molecule. In some embodiments, the therapeutic agent comprises a complex or conjugate of a nucleic acid and a lipid. In some embodiments, the therapeutic agent comprises a complex or conjugate of a nucleic acid and a small molecule. In some embodiments, the therapeutic agent comprises a complex or conjugate of a protein and small molecule.

In some embodiments, the therapeutic agent (e.g., BCS Class III or IV therapeutic agent) has a molecular weight of greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the therapeutic agent has a molecular weight of about 0.1 to about 1 kDa.

### Additional Therapeutic Agents

In some embodiments, the therapeutic agent (e.g., BCS Class TIT or IV therapeutic agent) comprises an oligosaccharide. In some embodiments, the therapeutic agent is an oligosaccharide. In some embodiments, the therapeutic agent (e.g., BCS Class III or IV therapeutic agent) comprises a lipid.

In some embodiments, the therapeutic agent (e.g., BCS Class III or IV therapeutic agent) comprises a small molecule. In some embodiments, the small molecule has a molecular weight of less than 1,000 g/mol. As used herein, the units “g/mol” are used synonymously with Daltons (Da). In some embodiments, the small molecule has a molecular weight of at least about 100 g/mol and up to about 1,000 g/mol. In some embodiments, the small molecule has a molecular weight (in g/mol) of less than or equal to about: 150, 200, 250, 300, 350, 400, 500, 600, 700, 800, 900, 1000, or ranges including and/or spanning the aforementioned values. In some embodiments, the small molecule is chemically synthesized (e.g., synthetic) by one or more chemical reactions.

In some embodiments, the small molecule is in a dosage form that is crystalline. In some embodiments, the small molecule is in a dosage form that is amorphous. As used herein, “amorphous” refers to a solid form of a molecule and/or ion that is not crystalline. As used herein, the term “small molecule” encompasses all forms thereof including optically pure enantiomers or mixtures, racemic or otherwise, of enantiomers as well as derivative forms such as, for example, salts, acids, esters and the like.

### Bile Acid or Salt Thereof

Bile acids or salts thereof are ionic amphiphilic compounds generally comprising a steroid skeleton, a carbon side-chain (e.g., a side chain comprising 4-8 carbon atoms), and the hydroxyl groups. The steroid skeleton comprises four rings labeled from left to right as ring A, B, C, and D. The D ring is smaller by one carbon the A, B, and C rings. The “beta” hydroxyl groups are positioned either up (i.e., conventionally drawn as a solid line) and the “alpha” hydroxyl groups are down (i.e., conventionally drawn by a dashed line).

Bile acids are often derived from cholesterol in the liver (see, e.g., Chiang J Y. J Hepatol. 2004; 40:539-551; Chiang J Y L. Front Biosci. 1998; 3:D176-D193; Russell D W. Annu Rev Biochem. 2003; 72:1370174). For example, the synthesis of bile acids or salts thereof is the major route for elimination of cholesterol from the body. Cholic acid (CA) and chenodeoxycholic acid (CDCA) are the major primary bile acids synthesized in human livers. CA and CDCA are conjugated with taurine or glycine for secretion in the bile. Bile salts form mixed micelles with phospholipids and cholesterol and are secreted into the GI tract to facilitate digestion and nutrient absorption.

In some embodiments, a bile acid or salt thereof of the disclosure is converted from cholesterol. The structure of cholesterol is depicted by Compound A. The structure has a beta-hydroxyl group at position 3 and the 4 steroid rings are flat. The human liver has two major bile acid biosynthetic pathways (see, e.g., Chiang, et al (2013) Compr Physiol 3:1191; Pellicoro, et al (2007) Alinent Pharmacol Ther 26 (Suppl 2) 149-160) that are the neutral bile acid pathway (also known as the “classic pathway”) and the acidic pathway. These pathways convert cholesterol to CA or CDCA by a combination of (i) hydroxylation of the steroid nucleus (e.g., installation of an alpha hydroxyl group at position 7); and (ii) oxidative cleavage of the side chain at position 17 of the steroid nucleus. The neutral bile acid pathway converts cholesterol to CA or CDCA by a series of enzymatic steps comprising (i) then (ii). The acidic pathway by a series of enzymatic steps comprising (ii) then (i). In the conversion, the junction between rings A and B is altered, resulting in conversion of the hydroxyl group at position 3 to the alpha orientation. The bile acid is further amidated at the carboxyl group with glycine or taurine.

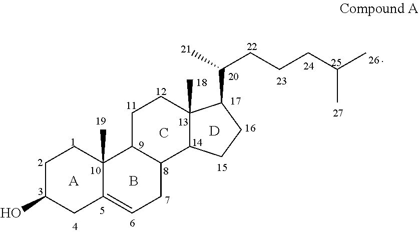

In some embodiments, a bile acid or salt thereof of the disclosure is converted from cholesterol by hydroxylation, saturation of the double bond at C5-C6, epimerization of the 3-hydroxyl group, oxidative cleavage of a 3-carbon unit from the side chain (i.e., C25-C27), or a combination thereof. In some embodiments, the bile acid or salt thereof is converted from cholesterol by one or more enzymes in the neutral bile acid pathway. In some embodiments, the bile acid or salt thereof is converted from cholesterol by one or more enzymes in the acidic bile acid pathway. In some embodiments, the bile acid or salt thereof comprises a carboxylate at position 24. In some embodiments, the bile acid or salt thereof comprises an ester at position 24. In some embodiments, the bile acid or salt thereof comprises an amide at position 24. In some embodiments, the bile acid or salt thereof is amidated with taurine at position 24. In some embodiments, the bile acid or salt thereof is amidated with glycine at position 24.

In some embodiments, a bile acid of the disclosure comprises a steroid nucleus. As used herein, a “steroid nucleus” refers to a perhydrocyclopentanophenanthrene nucleus shared by steroids, e.g., a compound of Formula (I) as further described herein. As shown below, the steroid nucleus comprises four rings: three six carbon rings (A, B and C), and one five carbon ring (D).

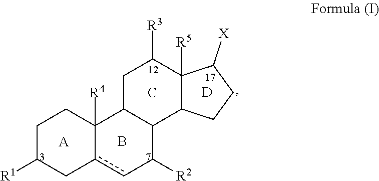

wherein: [symbol] R¹, R², R³, R⁴, R⁵, and X are further defined below.

In some embodiments, the bile acid comprises a steroid nucleus. In some embodiments, the bile acid comprises a steroid nucleus having one, two, or three hydroxy groups. In some embodiments, the bile acid comprises a steroid nucleus having one hydroxy group. In some embodiments, the one hydroxy group is beta or alpha. In some embodiments, the one hydroxy group is at any open position of the steroid nucleus (e.g., position 3, 7, or 12). In some embodiments, the one hydroxyl group is at position 3 of the steroid nucleus. In some embodiments, the one hydroxyl group at position 3 is beta. In some embodiments, the one hydroxyl group is at position 7 of the steroid nucleus. In some embodiments, the one hydroxyl group is at position 12 of the steroid nucleus.

In some embodiments, the bile acid comprises a steroid nucleus having first and a second hydroxy groups. In some embodiments, the first and the second hydroxy groups are at any open position of the steroid nucleus (e.g., position 3, 7, and/or 12). In some embodiments, the first and the second hydroxy groups are each independently alpha or beta. In some embodiments, the first hydroxy group is at position 3 and the second hydroxy group is at position 7 of the steroid nucleus. In some embodiments, the first hydroxy group at position 3 and the second hydroxy group at position 7 are both beta. In some embodiments, the first hydroxy group is at position 3 and the second hydroxy group is at position 12 of the steroid nucleus. In some embodiments, the first hydroxy group at position 3 and the second hydroxy group at position 12 are both beta. In some embodiments, the first hydroxy group is at position 7 and the second hydroxy group is at position 12 of the steroid nucleus. In some embodiments, the first hydroxy group at position 7 and the second hydroxy group at position 12 are both beta.

In some embodiments, the bile acid comprises a steroid nucleus having a first, second, and third hydroxy groups. In some embodiments, the first, second, and third hydroxy groups are at any open position of the steroid nucleus (e.g., position 3, 7, and/or 12). In some embodiments, the first, second, and third hydroxy groups are each independently alpha or beta. In some embodiment, the first, second, and third hydroxy groups are at positions 3, 7, and 12 respectively of the steroid nucleus. In some embodiment, the first, second, and third hydroxy groups at positions 3, 7, and 12 are each beta.

In some embodiments, the bile acid comprises a side chain at position 17 of the steroid nucleus. In some embodiments, the side chain comprises a carboxylate. In some embodiments, the side chain comprises an ester. In some embodiments, the side chain comprises an amide. In some embodiments, the side chain comprises a conjugate to any naturally occurring or synthetic amino acid known in the art. In some embodiments, the side chain comprises a conjugate to taurine. In some embodiments, the side chain comprises a conjugate to glycine.

In some embodiments, the composition comprises a pharmaceutically acceptable salt of the bile acid. In some embodiments, the composition comprises a sodium salt of the bile acid. In some embodiments, the composition comprises a potassium salt of the bile acid.

In some embodiments, a composition of the disclosure comprises one or more trihydroxy conjugated bile acid or salt thereof. Exemplary trihydroxy conjugated bile acids include, but are not limited to, glycocholate (GC), taurocholate (TC), glycohyocholate (GHC), taurohyocholate (THC), tauro-α-muricholate (T-α-MC), 3-[(3-Cholamidopropyl) dimethylammonio]-1-propanesulfonate (CHAPS), and tauro-β-muricholate (T-β-MC). In some embodiments, the composition comprises the trihydroxy conjugated bile acid. In some embodiments, the composition comprises a pharmaceutically acceptable salt of the trihydroxy conjugated bile acid. In some embodiments, the composition comprises a sodium salt of the trihydroxy conjugated bile acid. In some embodiments, the composition comprises a potassium salt of the trihydroxy conjugated bile acid.

In some embodiments, a composition of the disclosure comprises one or more dihydroxy conjugated bile acid or salt thereof. Exemplary dihydroxy conjugated bile acids include, but are not limited to, tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxycholate (GDC), and glycoursodeoxycholate (GUDC). In some embodiments, the composition comprises the dihydroxy conjugated bile acid. In some embodiments, the composition comprises a pharmaceutically acceptable salt of the dihydroxy conjugated bile acid. In some embodiments, the composition comprises a sodium salt of the dihydroxy conjugated bile acid. In some embodiments, the composition comprises a potassium salt of the dihydroxy conjugated bile acid.

In some embodiments, a composition of the disclosure comprises one or more unconjugated bile acid or salt thereof. Exemplary unconjugated bile acids include, but are not limited to, deoxycholate (DC), cholate, and chenodeoxycholate (CDC). In some embodiments, the composition comprises the unconjugated bile acid. In some embodiments, the composition comprises a pharmaceutically acceptable salt of the unconjugated bile acid. In some embodiments, the composition comprises a sodium salt of the unconjugated bile acid. In some embodiments, the composition comprises a potassium salt of the unconjugated bile acid.

### Exemplary Bile Acids or Salts Thereof

In some embodiments, the bile acid or salt thereof is a compound of Formula (I):

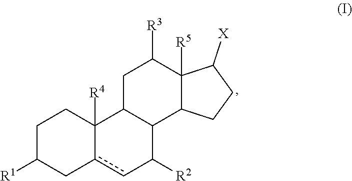

or a pharmaceutically acceptable salt, solvate, clathrate, hydrate, stereoisomer, or tautomer thereof, wherein:
   [symbol] denotes a single or double bond;
   R¹, R², and R³ each independently are H, halogen, —CN, —NO₂, —OH, —NH₂, —O(C₁-C₆ alkyl), —NH(C₁-C₆ alkyl), or —NH(C₁-C₆ alkyl)₂;
   R⁴ and R⁵ each independently are H, halogen, —CN, —NO₂, —OH, —NH₂, —O(C₁-C₆ alkyl), —NH(C₁-C₆ alkyl), —NH(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more oxo, halogen, —CN, —NO₂, —OH, or —NH₂;
   X is —(C₁-C₁₂ alkylene)-R^{X}, —(C₂-C₁₂ alkenylene)-R^{X}, or —(C₂-C₁₂ alkynylene)-R^{X}, wherein the C₁-C₁₂ alkylene, C₂-C₁₂ alkenylene or C₂-C₁₂ alkynylene is optionally substituted with one or more oxo, halogen, —CN, —NO₂, —OH, or —NH₂;
   R^{X} is —C(═O)R^{Xa}, —C(═O)OR^{Xa}, —OC(═O)R^{Xa}, —N(R^{Xa})C(═O)(R^{Xa}), —C(═O)N(R^{Xa})₂, —OS(═O)₂R^{Xa}, or —S(═O)₂OR^{Xa};
   each R^{Xa} independently is H, C₁-C₁₂ alkyl, C₂-C₁₂ alkenyl, or C₂-C₁₂ alkynyl, wherein the C₁-C₁₂ alkyl, C₂-C₁₂ alkenyl, or C₂-C₁₂ alkynyl is optionally substituted with one or more oxo, halogen, —CN, —NO₂, —OH, —NH₂, —C(═O)R^{Xb}, —C(═O)OR^{b}, —OC(═O)R^{Xb}, —N(R^{Xb})C(═O)(R^{Xb}), —N⁺(R^{Xb})₃, —C(═O)N(R^{Xb})₂, —OS(═O)₂R^{Xb}, or —S(═O)₂OR^{Xb}; and
   each R^{Xb} independently is H, C₁-C₁₂ alkyl, C₂-C₁₂ alkenyl, or C₂-C₁₂ alkynyl, wherein the C₁-C₁₂ alkyl, C₂-C₁₂ alkenyl, or C₂-C₁₂ alkynyl is optionally substituted with one or more oxo, halogen, —CN, —NO₂, —OH, —NH₂, —C(═O)OH, or —S(═O)₂OH.

### (i) Variables R¹, R², and R³

In some embodiments, R¹ is H.

In some embodiments, R¹ is halogen, —CN, —NO₂, —OH, —NH₂, —O(C₁-C₆ alkyl), —NH(C₁-C₆ alkyl), or —NH(C₁-C₆ alkyl)₂.

In some embodiments, R¹ is halogen (e.g., F, Cl, or Br). In some embodiments, R¹ is —CN. In some embodiments, R¹ is —NO₂. In some embodiments, R¹ is —OH. In some embodiments, R¹ is —NH₂. In some embodiments, R¹ is —O(C₁-C₆ alkyl). In some embodiments, R¹ is —NH(C₁-C₆ alkyl). In some embodiments, R¹ is —NH(C₁-C₆ alkyl)₂.

In some embodiments, R² is H.

In some embodiments, R² is halogen, —CN, —NO₂, —OH, —NH₂, —O(C₁-C₆ alkyl), —NH(C₁-C₆ alkyl), or —NH(C₁-C₆ alkyl)₂.

In some embodiments, R² is halogen (e.g., F, Cl, or Br). In some embodiments, R² is —CN. In some embodiments, R² is —NO₂. In some embodiments, R² is —OH. In some embodiments, R² is —NH₂. In some embodiments, R² is —O(C₁-C₆ alkyl). In some embodiments, R² is —NH(C₁-C₆ alkyl). In some embodiments, R² is —NH(C₁-C₆ alkyl)₂.

In some embodiments, R³ is H.

In some embodiments, R³ is halogen, —CN, —NO₂, —OH, —NH₂, —O(C₁-C₆ alkyl), —NH(C₁-C₆ alkyl), or —NH(C₁-C₆ alkyl)₂.

In some embodiments, R³ is halogen (e.g., F, Cl, or Br). In some embodiments, R³ is —CN. In some embodiments, R³ is —NO₂. In some embodiments, R³ is —OH. In some embodiments, R³ is —NH₂. In some embodiments, R³ is —O(C₁-C₆ alkyl). In some embodiments, R³ is —NH(C₁-C₆ alkyl). In some embodiments, R³ is —NH(C₁-C₆ alkyl)₂.

In some embodiments, at least one of R¹, R², and R³ is —OH. In some embodiments, R¹ is —OH. In some embodiments, R² is —OH. In some embodiments, R³ is —OH.

In some embodiments, one of R¹, R², and R³ is —OH. In some embodiments, R¹ is —OH, and R² and R³ each independently are H, halogen, —CN, —NO₂, —NH₂, —O(C₁-C₆ alkyl), —NH(C₁-C₆ alkyl), —NH(C₁-C₆ alkyl)₂. In some embodiments, R² is —OH, and R¹ and R³ each independently are H, halogen, —CN, —NO₂, —NH₂, —O(C₁-C₆ alkyl), —NH(C₁-C₆ alkyl), —NH(C₁-C₆ alkyl)₂. In some embodiments, R³ is —OH, and R¹ and R² each independently are H, halogen, —CN, —NO₂, —NH₂, —O(C₁-C₆ alkyl), —NH(C₁-C₆ alkyl), —NH(C₁-C₆ alkyl)₂.

In some embodiments, at least two of R¹, R², and R³ are —OH. In some embodiments, R¹ and R² each are —OH. In some embodiments, R² and R³ each are —OH. In some embodiments, R¹ and R³ each are —OH.

In some embodiments, two of R¹, R², and R³ are —OH. In some embodiments, R¹ and R² each are —OH, and R³ is H, halogen, —CN, —NO₂, —NH₂, —O(C₁-C₆ alkyl), —NH(C₁-C₆ alkyl), —NH(C₁-C₆ alkyl)₂. In some embodiments, R¹ and R³ each are —OH, and R² is H, halogen, —CN, —NO₂, —NH₂, —O(C₁-C₆ alkyl), —NH(C₁-C₆ alkyl), —NH(C₁-C₆ alkyl)₂. In some embodiments, R² and R³ each are —OH, and R¹ is H, halogen, —CN, —NO₂, —NH₂, —O(C₁-C₆ alkyl), —NH(C₁-C₆ alkyl), —NH(C₁-C₆ alkyl)₂.

In some embodiments, R¹, R², and R³ each are —OH.

### (ii) Variables R⁴ and R⁵

In some embodiments, R⁴ is H.

In some embodiments, R⁴ is halogen, —CN, —NO₂, —OH, —NH₂, —O(C₁-C₆ alkyl), —NH(C₁-C₆ alkyl), —NH(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more oxo, halogen, —CN, —NO₂, —OH, or —NH₂.

In some embodiments, R⁴ is halogen (e.g., F, Cl, or Br). In some embodiments, R⁴ is —CN. In some embodiments, R⁴ is —NO₂. In some embodiments, R⁴ is —OH. In some embodiments, R⁴ is —NH₂. In some embodiments, R⁴ is —O(C₁-C₆ alkyl), wherein the C₁-C₆ alkyl is optionally substituted with one or more oxo, halogen, —CN, —NO₂, —OH, or —NH₂. In some embodiments, R⁴ is —NH(C₁-C₆ alkyl), wherein the C₁-C₆ alkyl is optionally substituted with one or more oxo, halogen, —CN, —NO₂, —OH, or —NH₂. In some embodiments, R⁴ is —NH(C₁-C₆ alkyl)₂, wherein the C₁-C₆ alkyl is optionally substituted with one or more oxo, halogen, —CN, —NO₂, —OH, or —NH₂. In some embodiments, R⁴ is C₁-C₆ alkyl optionally substituted with one or more oxo, halogen, —CN, —NO₂, —OH, or —NH₂. In some embodiments, R⁴ is C₂-C₆ alkenyl optionally substituted with one or more oxo, halogen, —CN, —NO₂, —OH, or —NH₂. In some embodiments, R⁴ is C₂-C₆ alkynyl optionally substituted with one or more oxo, halogen, —CN, —NO₂, —OH, or —NH₂.

In some embodiments, R⁵ is H.

In some embodiments, R⁵ is halogen, —CN, —NO₂, —OH, —NH₂, —O(C₁-C₆ alkyl), —NH(C₁-C₆ alkyl), —NH(C₁-C₆ alkyl)₂, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more oxo, halogen, —CN, —NO₂, —OH, or —NH₂.

In some embodiments, R⁵ is halogen (e.g., F, Cl, or Br). In some embodiments, R⁵ is —CN. In some embodiments, R⁵ is —NO₂. In some embodiments, R⁵ is —OH. In some embodiments, R⁵ is —NH₂. In some embodiments, R⁵ is —O(C₁-C₆ alkyl), wherein the C₁-C₆ alkyl is optionally substituted with one or more oxo, halogen, —CN, —NO₂, —OH, or —NH₂. In some embodiments, R⁵ is —NH(C₁-C₆ alkyl), wherein the C₁-C₆ alkyl is optionally substituted with one or more oxo, halogen, —CN, —NO₂, —OH, or —NH₂. In some embodiments, R⁵ is —NH(C₁-C₆ alkyl)₂, wherein the C₁-C₆ alkyl is optionally substituted with one or more oxo, halogen, —CN, —NO₂, —OH, or —NH₂.

In some embodiments, R⁵ is C₁-C₆ alkyl optionally substituted with one or more oxo, halogen, —CN, —NO₂, —OH, or —NH₂. In some embodiments, R¹ is C₂-C₆ alkenyl optionally substituted with one or more oxo, halogen, —CN, —NO₂, —OH, or —NH₂. In some embodiments, R⁵ is C₂-C₆ alkynyl optionally substituted with one or more oxo, halogen, —CN, —NO₂, —OH, or —NH₂.

In some embodiments, R⁴ and R⁵ each independently are C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen, —CN, —NO₂, —OH, —NH₂, or oxo.

In some embodiments, R⁴ is C₁-C₆ alkyl (e.g., methyl, ethyl, or propyl) optionally substituted with one or more halogen, —CN, —NO₂, —OH, —NH₂, or oxo. In some embodiments, R⁵ is C₁-C₆ alkyl (e.g., methyl, ethyl, or propyl) optionally substituted with one or more halogen, —CN, —NO₂, —OH, —NH₂, or oxo. In some embodiments, R⁴ and R⁵ each independently are C₁-C₆ alkyl (e.g., methyl, ethyl, or propyl) optionally substituted with one or more halogen, —CN, —NO₂, —OH, —NH₂, or oxo. In some embodiments, R⁴ is methyl optionally substituted with one or more halogen, —CN, —NO₂, —OH, —NH₂, or oxo. In some embodiments, R⁵ is methyl optionally substituted with one or more halogen, —CN, —NO₂, —OH, —NH₂, or oxo. In some embodiments, R⁴ and R⁵ each independently are methyl optionally substituted with one or more halogen, —CN, —NO₂, —OH, —NH₂, or oxo.

In some embodiments, R⁴ is C₁-C₆ alkyl (e.g., methyl, ethyl, or propyl). In some embodiments, R⁵ is C₁-C₆ alkyl (e.g., methyl, ethyl, or propyl). In some embodiments, R⁴ and R⁵ each independently are C₁-C₆ alkyl (e.g., methyl, ethyl, or propyl). In some embodiments, R⁴ is methyl. In some embodiments, R⁵ is methyl. In some embodiments, R⁴ and R⁵ each are methyl.

### (iii) Variables X, R^{X}, R^{Xa}, and R^{Xb}

In some embodiments, X is —(C₁-C₁₂ alkylene)-R^{X}, —(C₂-C₁₂ alkenylene)-R^{X}, or —(C₂-C₁₂ alkynylene)-R^{X}, wherein the C₁-C₁₂ alkylene, C₂-C₁₂ alkenylene or C₂-C₁₂ alkynylene is optionally substituted with one or more oxo, halogen, —CN, —NO₂, —OH, or —NH₂.

In some embodiments, X is —(C₁-C₁₂ alkylene)-R^{X} optionally substituted with one or more oxo, halogen, —CN, —NO₂, —OH, or —NH₂. In some embodiments, X is —(C₂-C₁₂ alkenylene)-R^{X} optionally substituted with one or more oxo, halogen, —CN, —NO₂, —OH, or —NH₂. In some embodiments, X is —(C₂-C₁₂ alkynylene)-R^{X} optionally substituted with one or more oxo, halogen, —CN, —NO₂, —OH, or —NH₂.

In some embodiments, X is —(C₁-C₁₂ alkylene)-R^{X} (e.g., alkylene is methylene, ethylene, propylene, or butylene) optionally substituted with one or more oxo, halogen, —CN, —NO₂, —OH, or —NH₂. In some embodiments, X is —(C₁-C₁₂ alkylene)-R^{X} (e.g., alkylene is methylene, ethylene, propylene or butylene). In some embodiments, X is -(butylene)-R^{X} optionally substituted with one or more oxo, halogen, —CN, —NO₂, —OH, or —NH₂. In some embodiments, X is -(butylene)-R^{X}.

In some embodiments, R^{X} is —C(═O)OR^{Xa} or —C(═O)N(R^{Xa})₂. In some embodiments, R^{X} is —C(═O)R^{Xa}. In some embodiments, R^{X} is —C(═O)OR^{Xa}. In some embodiments, R^{X} is —OC(═O)R^{Xa}. In some embodiments, R^{X} is —N(R^{Xa})C(═O)(R^{Xa}). In some embodiments, R^{X} is —C(═O)N(R^{Xa})₂. In some embodiments, R^{X} is —OS(═O)₂R^{Xa}. In some embodiments, R^{X} is —S(═O)₂OR^{Xa}.

In some embodiments, at least one R^{Xa} is H. In some embodiments, at least one R^{Xa} is C₁-C₁₂ alkyl (e.g., methyl, ethyl, or propyl) optionally substituted with one or more oxo, halogen, —CN, —NO₂, —OH, —NH₂, —C(═O)R^{Xb}, —C(═O)OR^{Xb}, —OC(═O)R^{Xb}, —N(R^{Xb})C(═O)(R^{Xb})—N⁺(R^{Xb})₃, —C(═O)N(R^{X})₂, —OS(═O)₂R^{Xb}, or —S(═O)₂OR^{X}. In some embodiments, at least one R^{Xa} is C₂-C₁₂ alkenyl optionally substituted with one or more oxo, halogen, —CN, —NO₂, —OH, —NH₂, —C(═O)R^{Xb}, —C(═O)OR^{Xb}, —OC(═O)R^{Xb}, —N(R^{Xb})C(═O)(R^{Xb}), —N⁺(R^{Xb})₃, —C(═O)N(R^{Xb})₂, —OS(═O)₂R^{Xb}, or —S(═O)₂OR^{Xb}. In some embodiments, at least one R^{Xa} is C₂-C₁₂ alkynyl optionally substituted with one or more oxo, halogen, —CN, —NO₂, —OH, —NH₂, —C(═O)R^{Xb}, —C(═O)OR^{Xb}, —OC(═O)R^{Xb}, —N(R^{Xb})C(═O)(R^{Xb}), —N⁺(R^{Xb})₃, —C(═O)N(R^{Xb})₂, —OS(═O)₂R^{Xb}, or —S(═O)₂OR^{Xb}.

In some embodiments, at least one R^{Xa} is methyl optionally substituted with one or more oxo, halogen, —CN, —NO₂, —OH, —NH₂, —C(═O)R^{Xb}, —C(═O)OR^{Xb}, —OC(═O)R^{X}b, —N(R^{X})C(═O)(R^{Xb}), —N⁺(R^{Xb})₃, —C(═O)N(R^{Xb})₂, —OS(═O)₂R^{Xb}, or —S(═O)₂OR^{Xb}. In some embodiments, at least one R^{Xa} is methyl optionally substituted with one or more —C(═O)OR^{Xb}. In some embodiments, at least one R^{Xa} is methyl substituted with one or more —C(═O)OR^{Xb}. In some embodiments, at least one R^{Xa} is methyl substituted with —C(═O)OR^{Xb}.

In some embodiments, at least one R^{Xa} is ethyl optionally substituted with one or more oxo, halogen, —CN, —NO₂, —OH, —NH₂, —C(═O)R^{Xb}, —C(═O)OR^{Xb}, —OC(═O)R^{Xb}, —N(R b)C(═O)(R^{Xb}), —N⁺(R^{Xb})₃, —C(═O)N(R^{Xb})₂, —OS(═O)₂R^{Xb}, or —S(═O)₂OR^{Xb}. In some embodiments, at least one R^{Xa} is ethyl optionally substituted with one or more —S(═O)₂OR^{Xb}. In some embodiments, at least one R^{Xa} is ethyl substituted with one or more —S(═O)₂OR^{Xb}. In some embodiments, at least one R^{Xa} is ethyl substituted with —S(═O)₂OR^{Xb}.

In some embodiments, at least one R^{Xa} is propyl optionally substituted with one or more oxo, halogen, —CN, —NO₂, —OH, —NH₂, —C(═O)R^{Xb}, —C(═O)OR^{Xb}, —OC(═O)R^{Xb}, —N(R^{Xb})C(═O)(Rb), —N⁺(R^{Xb})₃, —C(═O)N(R^{Xb})₂, —OS(═O)₂R^{Xb}, or —S(═O)₂OR^{Xb}. In some embodiments, at least one R^{Xa} is propyl optionally substituted with one or more —N⁺(R^{Xb})₃. In some embodiments, at least one R^{Xa} is propyl substituted with one or more —N⁺(R^{Xb})₃. In some embodiments, at least one R^{Xa} is propyl substituted with —N⁺(R^{Xb})₃.

In some embodiments, at least one R^{Xb} is H. In some embodiments, at least one R^{Xb} is C₁-C₁₂ alkyl optionally substituted with one or more oxo, halogen, —CN, —NO₂, —OH, —NH₂, —C(═O)OH, or —S(═O)₂OH. In some embodiments, at least one R^{Xb} is C₂-C₁₂ alkenyl optionally substituted with one or more oxo, halogen, —CN, —NO₂, —OH, —NH₂, —C(═O)OH, or —S(═O)₂OH. In some embodiments, at least one R^{Xb} is C₂-C₁₂ alkynyl optionally substituted with one or more oxo, halogen, —CN, —NO₂, —OH, —NH₂, —C(═O)OH, or —S(═O)₂OH.

In some embodiments, each R^{Xb} is H.

In some embodiments, at least one R^{Xb} is methyl optionally substituted with one or more oxo, halogen, —CN, —NO₂, —OH, —NH₂, —C(═O)OH, or —S(═O)₂OH. In some embodiments, at least two R^{Xb} are methyl optionally substituted with one or more oxo, halogen, —CN, —NO₂, —OH, —NH₂, —C(═O)OH, or —S(═O)₂OH. In some embodiments, at least two R^{Xb} are methyl.

In some embodiments, two R^{Xb} are methyl optionally substituted with one or more oxo, halogen, —CN, —NO₂, —OH, —NH₂, —C(═O)OH, or —S(═O)₂OH. In some embodiments, two R^{Xb} are methyl.

In some embodiments, at least one R^{Xb} is propyl optionally substituted with one or more oxo, halogen, —CN, —NO₂, —OH, —NH₂, —C(═O)OH, or —S(═O)₂OH. In some embodiments, at least one R^{Xb} is propyl substituted with one or more oxo, halogen, —CN, —NO₂, —OH, —NH₂, —C(═O)OH, or —S(═O)₂OH. In some embodiments, at least one R^{Xb} is propyl substituted with one or more —S(═O)₂OH. In some embodiments, at least one R^{Xb} is propyl substituted with one —S(═O)₂OH.

In some embodiments, one R^{Xb} is C₁-C₁₂ alkyl optionally substituted with one or more oxo, halogen, —CN, —NO₂, —OH, —NH₂, —C(═O)OH, or —S(═O)₂OH. In some embodiments, one R^{Xb} is propyl optionally substituted with one or more oxo, halogen, —CN, —NO₂, —OH, —NH₂, —C(═O)OH, or —S(═O)₂OH. In some embodiments, one R^{Xb} is propyl substituted with one or more oxo, halogen, —CN, —NO₂, —OH, —NH₂, —C(═O)OH, or —S(═O)₂OH. In some embodiments, one R^{Xb} is propyl substituted with one or more —S(═O)₂OH. In some embodiments, one R^{Xb} is propyl substituted with one —S(═O)₂OH.

### (iv) Exemplary Embodiments of the Compounds

In some embodiments, the compound is of Formula (I-1-a):

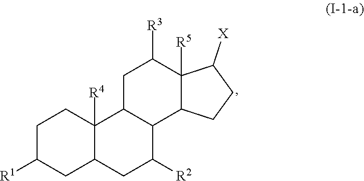

or a pharmaceutically acceptable salt, solvate, clathrate, hydrate, stereoisomer, or tautomer thereof.

In some embodiments, the compound is of Formula (I-1-b):

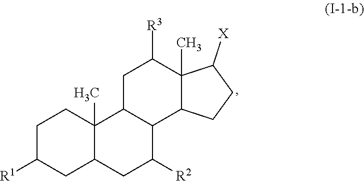

or a pharmaceutically acceptable salt, solvate, clathrate, hydrate, stereoisomer, or tautomer thereof.

In some embodiments, the compound is of Formula (I-1-c), (I-1-d), (I-1-e):

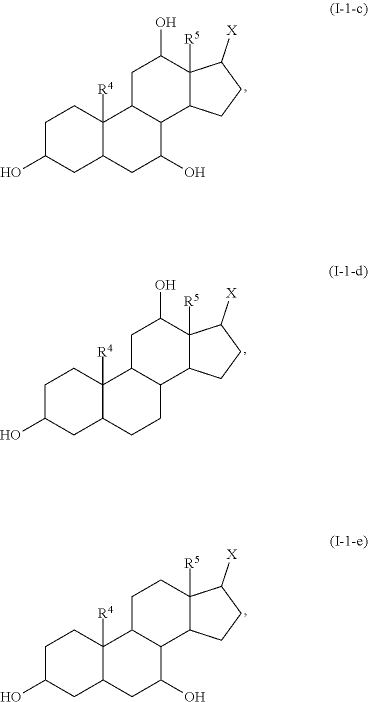

or a pharmaceutically acceptable salt, solvate, clathrate, hydrate, stereoisomer, or tautomer thereof.

In some embodiments, the compound is of Formula (I-1-f):

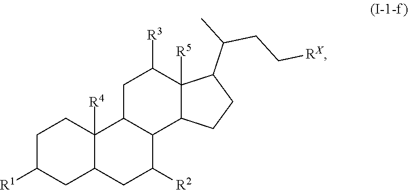

or a pharmaceutically acceptable salt, solvate, clathrate, hydrate, stereoisomer, or tautomer thereof.

In some embodiments, the compound is of Formula (I-2-a):

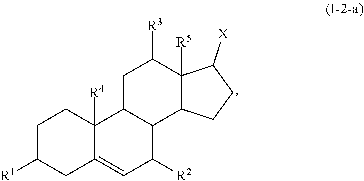

or a pharmaceutically acceptable salt, solvate, clathrate, hydrate, stereoisomer, or tautomer thereof.

In some embodiments, the compound is of Formula (I-2-b):

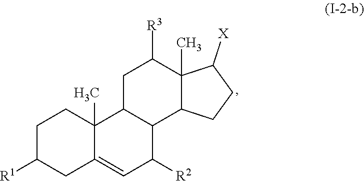

or a pharmaceutically acceptable salt, solvate, clathrate, hydrate, stereoisomer, or tautomer thereof.

In some embodiments, the compound is of Formula (I-2-c), (I-2-d), or (I-2-e):

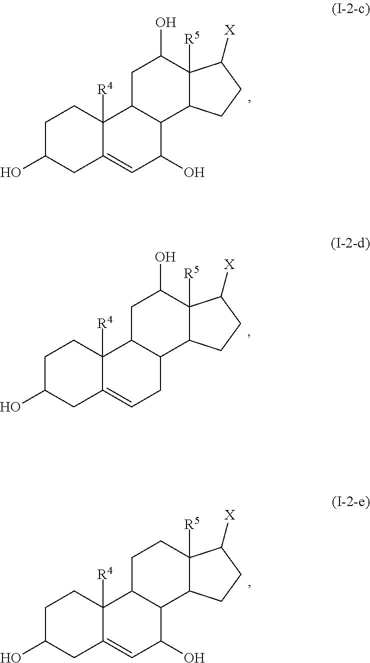

or a pharmaceutically acceptable salt, solvate, clathrate, hydrate, stereoisomer, or tautomer thereof.

In some embodiments, the compound is of Formula (I-2-f):

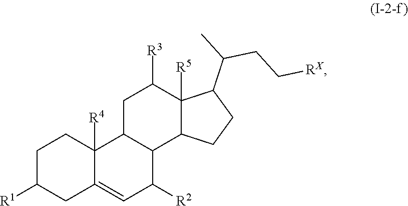

or a pharmaceutically acceptable salt, solvate, clathrate, hydrate, stereoisomer, or tautomer thereof.

In some embodiments, the compound is of Formula (I-1-a′):

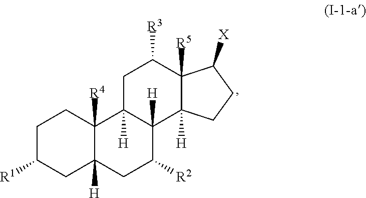

or a pharmaceutically acceptable salt, solvate, clathrate, hydrate, stereoisomer, or tautomer thereof.

In some embodiments, the compound is of Formula (I-1-b′):

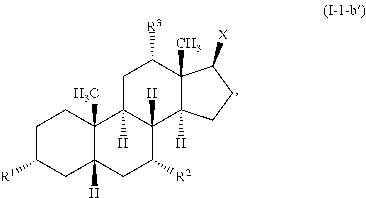

or a pharmaceutically acceptable salt, solvate, clathrate, hydrate, stereoisomer, or tautomer thereof.

In some embodiments, the compound is of Formula (I-1-c′), (I-1-d′), (I-1-e′):

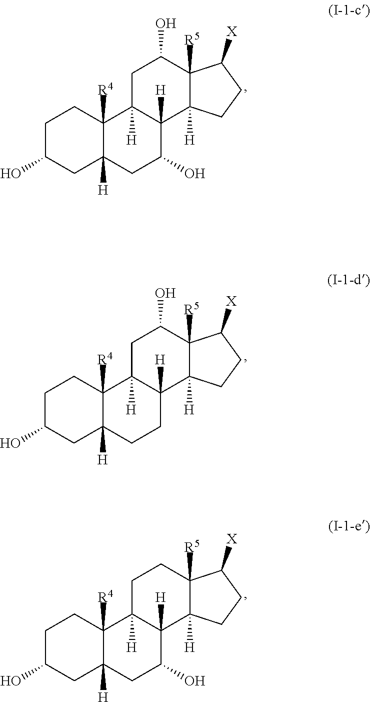

or a pharmaceutically acceptable salt, solvate, clathrate, hydrate, stereoisomer, or tautomer thereof.

In some embodiments, the compound is of Formula (I-1-f′):

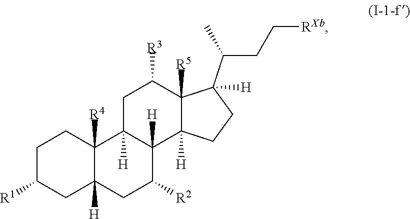

or a pharmaceutically acceptable salt, solvate, clathrate, hydrate, stereoisomer, or tautomer thereof.

In some embodiments, the compound is of Formula (I-2-a′):

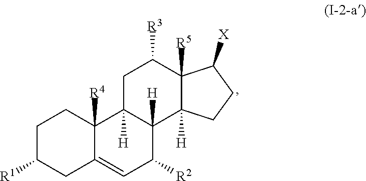

or a pharmaceutically acceptable salt, solvate, clathrate, hydrate, stereoisomer, or tautomer thereof.

In some embodiments, the compound is of Formula (I-2-b′):

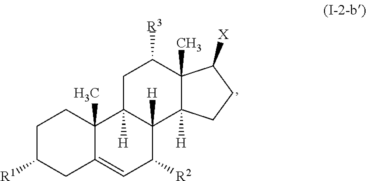

or a pharmaceutically acceptable salt, solvate, clathrate, hydrate, stereoisomer, or tautomer thereof.

In some embodiments, the compound is of Formula (I-2-c′), (I-2-d′), or (I-2-e′):

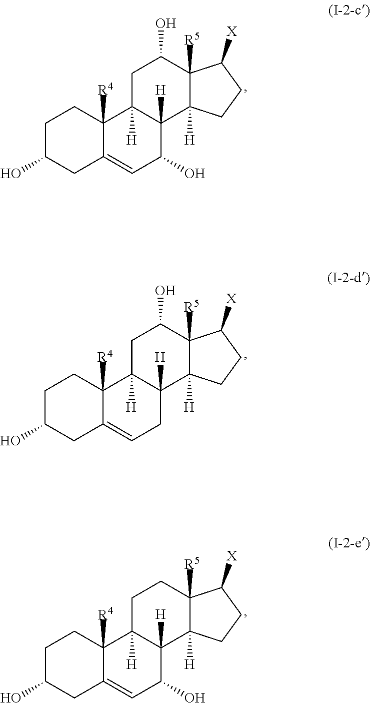

or a pharmaceutically acceptable salt, solvate, clathrate, hydrate, stereoisomer, or tautomer thereof.

In some embodiments, the compound is of Formula (I-2-f′):

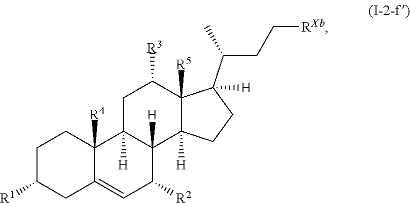

or a pharmaceutically acceptable salt, solvate, clathrate, hydrate, stereoisomer, or tautomer thereof.

In some embodiments, the compound is selected from the compounds described in Table A, and pharmaceutically acceptable salts, solvates, clathrates, hydrates, stereoisomers, and tautomers thereof.

In some embodiments, the compound is selected from the compounds described in Table A, and pharmaceutically acceptable salts thereof.

In some embodiments, the compound is selected from the compounds described in Table A.

TABLE A

| Compound No. | Compound Structure |
|---|---|
| 1 | 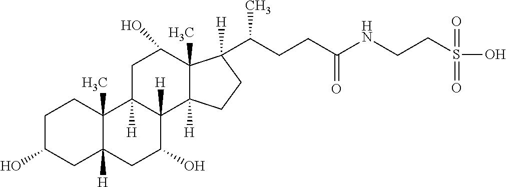 |
| 2 | 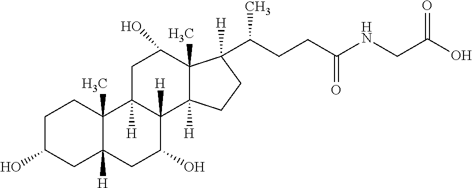 |
| 3 | 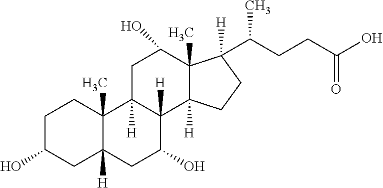 |
| 4 | 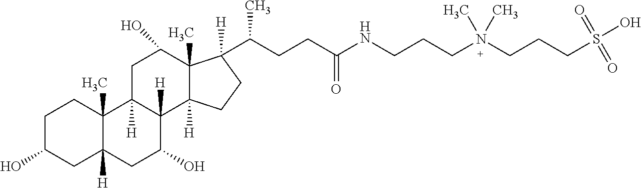 |
| 5 | 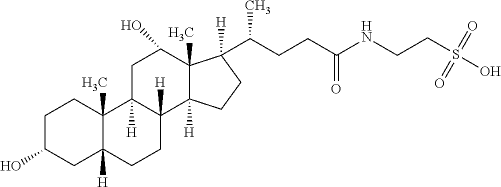 |
| 6 | 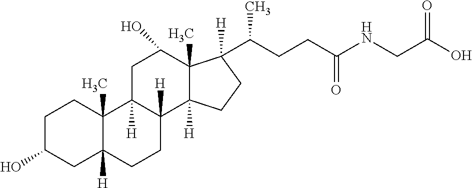 |
| 7 | 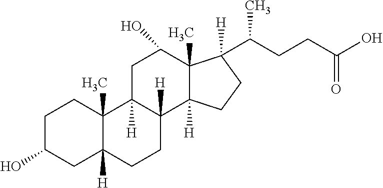 |
| 8 | 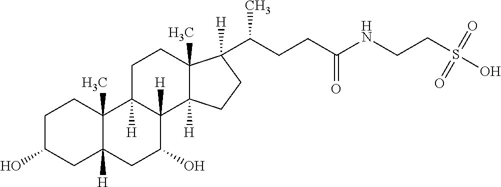 |
| 9 | 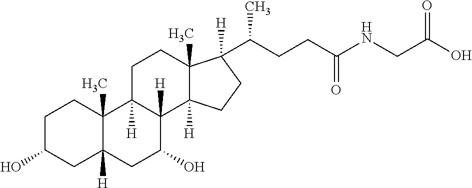 |
| 10 | 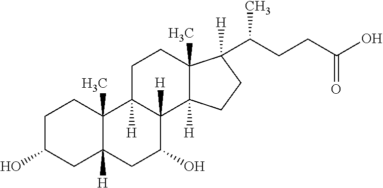 |

### (v) Definitions

The term “independently” is used herein to indicate that the variable, such as atom or functional group, which is independently applied, varies independently from application to application. For example, where more than one substituent or atom (carbon or heteroatom, such as oxygen (O), sulfur (S), or nitrogen (N)) occurs, each substituent or atom is independent of another substituent or atom and such substituents or atom can also alternate.

The term “alkyl”, as used herein, refers to saturated, straight-chain or branched hydrocarbon radicals containing, in certain embodiments, between one and twenty, including between one and ten, or between one and six, carbon atoms. Branched means that one or more lower C₁-C₆ alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. Examples of C₁-C₆ alkyl radicals include, but are not limited to, methyl, ethyl, propyl, isopropyl, butyl, tert-butyl, neopentyl, n-hexyl radicals; and examples of C₁—Cs alkyl radicals include, but are not limited to, methyl, ethyl, propyl, isopropyl, n-butyl, tert-butyl, neopentyl, n-hexyl, heptyl, octyl radicals. Examples of C₁-C₂₀ alkyl radicals include but are not limited to hexadecamethyl, hexadecaethyl, hexadecopropyl, octadecamethyl, octadecaethyl, octadecapropyl and the like.

The term “alkenyl”, as used herein, denotes a monovalent straight or branched group derived from a hydrocarbon moiety containing, in certain embodiments, from two to six, or two to eight, or two to twenty carbon atoms having at least one carbon-carbon double bond. The double bond may or may not be the point of attachment to another group. Examples of C₂-C₈ alkenyl groups include, but are not limited to, for example, ethenyl, propenyl, butenyl, 1-methyl-2-buten-1-yl, heptenyl, octenyl and the like. As defined herein, “alkenyl” groups include both cis- and trans-isomers.

The term “alkynyl”, as used herein, denotes a monovalent straight or branched group derived from a hydrocarbon moiety containing, in certain embodiments, from two to six, or two to eight, or two to twenty carbon atoms having at least one carbon-carbon triple bond. The triple bond may or may not be the point of attachment to another group. Examples of C₂-C₈ alkynyl groups include, but are not limited to, for example, ethynyl, propynyl, butynyl and the like.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

Similarly, the terms “alkenylene,” “alkynylene,” “cycloalkylene,” “arylene,” “heteroarylene,” and “heterocyclene” denote a divalent radical derived from the monovalent alkenyl, alkynyl, cycloalkyl, aryl, heteroaryl, or heterocyclene, respectively, by the abstraction of a second hydrogen atom from the respective monovalent radical.

The terms “hal”, “halo”, or “halogen”, as used herein, refer to an atom selected from fluorine, chlorine, bromine and iodine.

As used herein, the term “oxo” is understood to describe a carbonyl group (i.e., C(O)).

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds disclosed herein include the compounds themselves, as well as their salts, and their solvates if applicable. A salt, for example, can be formed between an anion and a positively charged group (e.g., protonated amino) on a compound of this disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of this disclosure. Suitable cations include sodium ion, potassium ion, magnesium ion, calcium ion, and an ammonium cation such as tetramethylammonium ion. The compounds of this disclosure also include those salts containing quaternary nitrogen atoms.

Additionally, physiologically acceptable, i.e. pharmaceutically compatible, salts can be salts of the compounds disclosed herein with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine or methylpiperidine.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

Some of the compounds of the present disclosure may exist in unsolvated as well as solvated forms such as, for example, hydrates.

“Solvate” means a solvent addition form that contains either a stoichiometric or non-stoichiometric amounts of solvent. Some compounds can have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the disclosure.

### Critical Micelle Concentration

In some embodiments, a bile acid or salt thereof of the disclosure is characterized by a critical micelle concentration (CMC). In some embodiments, the bile acid or salt thereof aggregates and/or forms micelles at a concentration above the CMC. Methods for measuring the CMC of a bile salt described herein are known in the art, see, e.g., Natalini, et al J Pharm Biomed Anal. (2014) 87:62-81. In some embodiments, the method comprises potentiometry, spectrophotometry, nuclear magnetic resonance, viscosimetry, turbidimetry, microcalorimetry, refractometry, conductimetry, cholesterol solubilization, bilirubin monoglucuronide solubilization, dye solubilization, fluorescence, capillary electrophoresis, reverse phase HPLC, freezing point depression, surface tension, and/or light scattering. In some embodiments, the CMC value is an average of at least 3-50 replicates measured by a single technique (e.g., 3-50 replicates measured by potentiometry, 3-50 replicates measured by spectrophotometry, or 3-50 replicates measured by light scattering). In some embodiments, the CMC value is an average of replicates measured by more than one technique, wherein at least 3, 4, 5, 6, 7, 8, 9, or 10 replicates are measured per technique (e.g., an average of (i) at least 3, 4, 5, 6, 7, 8, 9, or 10 replicates measured by potentiometry, (ii) at least 3, 4, 5, 6, 7, 8, 9, or 10 replicates measured by spectrophotometry, (iii) at least 3, 4, 5, 6, 7, 8, 9, or 10 replicates measured by light scattering, (iv) replicates from any pair-wise combination of (i)-(iii), or (v) replicates from a combination of (i)-(iii)).

CMC values of exemplary bile salts of the disclosure and measured according to such methods are set forth in Table 2.

In some embodiments, a bile acid or salt thereof described herein is characterized by a CMC of at least about 1 mM, about 2 mM, about 3 mM, about 4 mM, about 5 mM, about 6 mM, about 7 mM, about 8 mM, about 9 mM, or about 10 mM, e.g., as measured using a method described herein. In some embodiments, the bile acid or salt thereof is characterized by a CMC of not more than about 30 mM, about 25 mM, about 20 mM, about 18 mM, about 16 mM, about 14 mM, or about 12 mM, e.g., as measured using a method described herein. In some embodiments, the bile acid or salt thereof is characterized by a CMC of about 1 mM to about 10 mM, about 2 mM to about 10 mM, about 3 mM to about 10 mM, about 4 mM to about 10 mM, about 5 mM to about 10 mM, about 5 mM to about 15 mM, or about 1 mM, about 2 mM, about 3 mM, about 4 mM, about 5 mM, about 6 mM, about 7 mM, about 8 mM, about 9 mM, or about 10 mM, e.g., as measured using a method described herein.

In some embodiments, the bile acid or salt thereof is characterized by a CMC of at least about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, or about 5 mg/mL, e.g., as measured using a method described herein. In some embodiments, the bile acid or salt thereof is characterized by a CMC of not more than about 15 mg/mL, about 14 mg/mL, about 13 mg/mL, about 12 mg/mL, about 11 mg/mL, about 10 mg/mL, about 9 mg/mL, about 8 mg/mL, about 7 mg/mL, or about 6 mg/mL, e.g., as measured using a method described herein. In some embodiments, the bile acid or salt thereof is characterized by a CMC of about 0.1 mg/mL to about 10 mg/mL, of about 0.5 mg/mL to about 10 mg/mL, of about 1 mg/mL to about 10 mg/mL, of about 0.1 mg/mL to about 5 mg/mL, of about 0.5 mg/mL to about 5 mg/mL, or of about 1 mg/mL to about 5 mg/mL, e.g., as measured using a method described herein.

### Co-Excipients

In some embodiments, the disclosure provides a composition comprising a therapeutic agent, at least one bile acid or salt thereof described herein, and one or more co-excipients. As used herein, the term “co-excipient” refers to a component of the composition that is not the therapeutic agent or a bile acid or salt thereof. In some embodiments, the one or more co-excipients enhances the intestinal permeation of the therapeutic agent in a subject (e.g., a human subject) following oral administration, e.g., as compared to intestinal permeation of a control composition comprising (i) the therapeutic agent without the one or more co-excipients, or (ii) the therapeutic agent and the at least one bile acid or salt thereof without the one or more co-excipients. In some embodiments, the one or more co-excipients is selected from a chelator, a polymer, a dendrimer, a nanoparticle, a lipid, an alkyl acid, and a combination thereof.

In some embodiments, the one or more co-excipients provides additional properties for promoting systemic delivery of the therapeutic agent following oral administration. For example, in some embodiments, the one or more co-excipients are selected from bulking agents, binders, surfactants, bioadhesives, lubricants, disintegrants, stabilizers, solubilizers, glidants, and additives or factors that affect dissolution or disintegration time. In some embodiments, the one or more co-excipients is effective for providing enhanced mucosal penetration, protection of the therapeutic agent from gastric proteases, enhanced cell uptake.

### Chelators

In some embodiments, the disclosure provides a composition comprising a therapeutic agent, at least one bile acid or salt thereof described herein, and one or more chelators.

Chelating agents or chelators, also known as sequestrants, are molecules that have the ability to form stable complexes with metal ions, particularly di-valent and tri-valent metal ions including trace metals and heavy metals (e.g., calcium, magnesium, zinc, iron, chromium, and lead). As used herein, a “chelator”, “chelating agent”, “chelant”, “sequestering agent”, or “sequestrant” are interchangeable used to refer to polydentate (i.e., multiple bonded) ligand that forms two or more separate coordinate bonds with a single central atom.

In some embodiments, the chelator forms a complex with a copper salt (e.g., a copper (I) or copper (II) salt). In some embodiments, the chelator forms a complex with an iron salt (e.g., an iron (II) salt or an iron (III) salt. In some embodiments, the chelator forms a complex with a zinc salt (e.g., a zinc (II) salt).

In some embodiments, the chelator is selected from mannitol, sorbitol, saccharose, sucrose, trehalose, calcium phosphate (e.g., basic calcium phosphate, calcium hydrogen phosphate, dicalcium phosphate hydrate, disodium phosphate dihydrate), an amino acid (e.g., any one of the 20 standard proteinogenic a-amino acids), EDTA, EGTA, citrate, a complexing peptide (such as GHK, i.e., glycyl-histidyl-lysine peptide), polyacrylic acid, a polyacrylic acid derivative, a carbomer, a carbomer derivative, sodium alginate, a silicate (e.g., kaolin), hydroxypropyl methylcellulose (HPMC), methylcellulose (MC), glycerol, sodium dodecyl sulfate, calcium sulfate, calcium carbonate, and pharmaceutically acceptable salts of any of the aforementioned agents. In some embodiments, the chelator is an analog or derivative of the foregoing agents.

In some embodiments, the chelator is one described in: Fernane F et al., Interactions between calcium phosphate and heavy metal ions in aqueous solution, MATEC Web of Conferences, Vol. 5, EDP Sciences, 2013; U.S. Pat. No. 8,193,291; and Giertsen E et al. Caries Res. 1989; 23(4):278-83.

In some embodiments, the chelator comprises a polyol (such as, e.g., mannitol, sorbitol, or glycerol), a sugar-based surfactant (such as, e.g., any one of the surfactants referred to in Ferlin N et al., J Surfact Deterg. 2012; 15(3):259-264), a carboxylate (such as, e.g., citrate, tartrate, gluconate, oxalate, malate or salcaprozate), a phosphate (such as, e.g., sodium tri-phosphate or tetra potassium pyrophosphate), a sulfate (such as, e.g., sodium dodecyl sulfate), an amine (such as, e.g., ethylene diamine, chlorophyll, or choline), an amino acid or a complexing peptide (such as, e.g., lysine, glycine, histidine, or any of the amino acids or peptides referred to in Kober P A et al., Journal of Biological Chemistry. 1912; 13(1):1-13 or in Trzaskowski B et al., J Biol Inorg Chem. 2008; 13(1):133-7), an aminopolycarboxylic acid (such as, e.g., EDTA, EGTA, pentetic acid or diethylenetriaminepentaacetic acid (DTPA), or humic acid), a polymeric complexing agent (such as, e.g., HPMC (which may also be in the form of an HPMC capsule, such as QualiV, VCaps or VCaps plus), pullulan (which may also be in the form of a Pullulan capsule), a polycarboxylate, a carbomer or Carbopol, chitosan, alginate, povidone, polyvinylalcohol (PVA), or any complexing agent or polymer referred to in U.S. Pat. No. 8,193,291), a cyclodextrine, or any mixture of two or more of the aforementioned agents.

In some embodiments, the chelator comprises ethylenediaminetetraacetic acid (EDTA) or a salt thereof. In some embodiments, the chelator comprises the free acid of EDTA. In some embodiments, the chelator comprises a salt of EDTA (e.g., disodium EDTA, tetrasodium EDTA, dipotassium EDTA, calcium disodium EDTA).

In some embodiments, the chelator comprises ethyleneglycol-bis(β-aminoethyl)-N,N,N′,N′-tetraacetic acid (EGTA) or a salt thereof. In some embodiments, the chelator comprises the free acid of EGTA. In some embodiments, the chelator comprises a salt of EGTA (e.g., disodium EGTA, tetrasodium EGTA, dipotassium EGTA).

In some embodiments, the chelator comprises glutamate. In some embodiments, the chelator comprises any known salt of glutamate. In some embodiments, the chelator comprises sodium glutamate.

### Polymers

In some embodiments, the disclosure provides a composition comprising a therapeutic agent, at least one bile acid or salt thereof described herein, and one or more polymers. In some embodiments, the composition comprises a therapeutic agent, at least one bile acid or salt thereof described herein, and one or more hydrophilic polymers. In some embodiments, the composition comprises a therapeutic agent, at least one bile acid or salt thereof described herein, and one or more hydrophobic polymers. In some embodiments, the composition comprises a therapeutic agent, at least one bile acid or salt thereof described herein, one or more hydrophilic polymers and one or more hydrophobic polymers.

In some embodiments, the polymer is a non-biodegradable polymer. In some embodiments, the polymer is a biodegradable polymer. As used herein, the term “biodegradable” refers to polymers that dissolve or degrade within a period that is acceptable in the desired application (e.g., vivo therapy), typically less than about five years, and more preferably less than about one year, once exposed to a physiological solution of pH between about 6-8 and at a temperature of between about 25° C.-38° C.

In some embodiments, the polymer is naturally occurring (i.e., extracted or derived from a natural source). In some embodiments, the polymer is synthetic (i.e., synthesized from one or more chemical reactions). In some embodiments, the polymer is naturally occurring and further modified by one or more chemical reactions.

In some embodiments, the polymer is a naturally occurring polymer selected from albumin, collagen, gelatin and prolamines, for example, zein, and polysaccharides such as alginate, cellulose derivatives and polyhydroxyalkanoates, for example, polyhydroxybutyrate. In some embodiments, the polymer comprises cellulose or a derivative thereof (e.g., methylcellulose, carboxymethyl cellulose, hydroxyethyl cellulose, hydroxyethylmethyl cellulose). In some embodiments, the polymer comprises a natural gum (e.g., guar gum, xanthan gum, locust bean gum, karaya gum, veegum etc). In some embodiments, the polymer comprises an alginate. In some embodiments, the polymer comprises dextran. In some embodiments, the polymer comprises a polysaccharide.

In some embodiments, the polymer is a synthetic polymer selected from poly(hydroxy acids) (e.g., poly(lactic acid), poly(glycolic acid), and poly(lactic acid-co-glycolic acid)), poly(lactide), poly(glycolide), poly(lactide-co-glycolide), polyanhydrides, polyorthoesters, polyamides, polycarbonates, polyalkylenes (e.g., polyethylene and polypropylene), polyalkylene glycols (e.g., poly(ethylene glycol)), polyalkylene oxides (e.g., poly(ethylene oxide)), polyalkylene terepthalates (e.g., poly(ethylene terephthalate)), polyvinyl alcohols, polyvinyl ethers, polyvinyl esters, polyvinyl halides (e.g., polyvinyl chloride), polyvinylpyrrolidone, polysiloxanes, polyvinyl alcohols, polyvinyl acetate, polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” refers to polymers having substitutions, additions of chemical groups and other modifications routinely made by those skilled in the art.

In some embodiments, the polymer comprises a polyacrylates (e.g., carbopol, polycarbophil). In some embodiments, the polymer comprises a polyoxyethylene. In some embodiments, the polymer comprises polyethylene glycols (PEG). In some embodiments, the PEG has a molecular weight between 1000 Da and 40,000 Da. In some embodiments, the PEG is branched. In some embodiments, the PEG is linear. In some embodiments, the polymer comprises a polycarbophil (e.g., Noveon AA-I, Noveon CA-I, Noveon CA-2). In some embodiments, the polymer comprises poly(alkylmethacrylate). In some embodiments, the polymer comprises poly(vinyl acetate).

In some embodiments, the polymer is a homopolymer. In some embodiments, the polymer is a copolymer. In some embodiments, the copolymer is a block co-polymer. In some embodiments, the block copolymer comprises lactic acid and glycolic acid (e.g., poly(dl-lactide-co-glycolide) (PLGA), poly(lactic acid) (PLA), poly(glycolic acid) (PGA)). In some embodiments, the block copolymer comprises PEG and polypropylene (e.g., Pluronics, Tektronix or Genapol block copolymers). In some embodiments, the polymer comprises a combination of one or more block copolymers (e.g., PEG-PLA, or PEG-PLGA copolymers). In some embodiments, the copolymer is a random copolymer. In some embodiments, the copolymer is a graft copolymer. In some embodiments, the copolymer is an alterante copolymer.

In some embodiments, the homopolymer or copolymer comprises poly(methacrylate), poly(ethyl acrylate), poly(ethylene glycol), hyaluronic acid, polysaccharide, chitosan, arginine, poly(2-hydroxyethylmethacrylate), poly(N-isopropyl acrylamide), poly(ethylenimine), poly(N-(2-hydroxypropyl) methacrylamide), poly(glycolic acid), poly(lactic acid), poly(lactic-co-glycolic acid), poly(caprolactone), poly(orthoester), poly(anhydride), poly(amide), poly(ester amide), poly(phosphoester), poly(alky cyanoacrylate), or a combination thereof.

In some embodiments, the polymer has a molecular weight of at least about 0.5 kDa, about 1 kDa, about 1.5 kDa, about 2 kDa, about 2.5 kDa, about 3 kDa, about 3.5 kDa, about 4 kDa, about 4.5 kDa, about 5 kDa, about 10 kDa, about 15 kDa, about 20 kDa, about 30 kDa, about 40 kDa, about 50 kDa, about 60 kDa, about 70 kDa, about 80 kDa, about 90 kDa, or about 100 kDa. In some embodiments, the polymer has a molecular weight of about 0.5 kDa to about 10 kDa, about 1 kDa to about 10 kDa, about 5 kDa to about 10 kDa, about 0.5 kDa to about 20 kDa, about 1 kDa to about 20 kDa, about 5 kDa to about 20 kDa, about 10 kDa to about 20 kDa, about 0.5 kDa to about 50 kDa about 1 kDa to about 50 kDa about 5 kDa to about 50 kDa about 10 kDa to about 50 kDa, about 0.5 kDa to about 100 kDa about 1 kDa to about 100 kDa about 5 kDa to about 100 kDa or about 10 kDa to about 100 kDa.

In some embodiments, the homopolymer or copolymer comprises acrylic acid or an ester thereof and/or methacrylic acid or an ester thereof. In some embodiments, the homopolymer or copolymer comprises poly(acrylic acid), polyacrylate, poly(methacrylic acid), and poly(methacrylate) polymers, e.g., those sold under the Eudragit® trade name (Rohm GmbH, Darmstadt, Germany), e.g., Eudragit® L, Eudragit® S, Eudragit® RL, Eudragit® RS coating materials and mixtures thereof.

In some embodiments, the homopolymer or copolymer is a bioadhesive or mucoadhesive agent. The mucoadhesive hydrogel-forming polymers are hydrophilic and swellable, containing numerous hydrogen bond-forming groups, like hydroxyl, thiol, carboxyl or amine, which favor adhesion. Upon contact with the mucosal surface they may interact with moieties on the biological interface that result in polymer/mucus interaction (adhesion) via hydrogen bonding, electrostatic, hydrophobic or van der Waals interaction. In addition, when used in a dry form, they can absorb water from the mucosal surface and swell. Exemplary hydrogel-forming excipients are selected from PEGs and other polymers having an ethylene glycol (EG) backbone (e.g., EG homopolymers or cross-linked heteropolymers thereof), block copolymers of EG units (e.g., PEG homopolymers (such as Polyox Nl 0/MW=100,0001 Polyox-80/MW=200,000; Polyox 1105/MW=900,000; Polyox-301/MW=4,000,000; Polyox-303/MW=7,000,000, Polyox WSR—N-60K, all of which are tradenames of Union Carbide), hydroxypropylmethylcellylose (HPMC) of all molecular weights and grades, Poloxamers (such as Lutrol F-68, Lutrol F-127, F-105 etc, all tradenames of BASF Chemicals), Genapol, PEGs (e.g., PEG-1500, PEG-3500, PEG-4000, PEG-6000, PEG-8000, PEG-12000, PEG-20,000, etc.), natural gums (Xanthan gum, Locust bean gum, etc) and cellulose derivatives (HC, HMC, HMPC, HPC, CP, CMC), polyacrylic acid-based polymers either as free or cross-linked and combinations thereof, biodegradable polymers such as poly lactic acids, polyglycolic acids and any combination thereof, whether a physical blend or cross-linked. In some embodiments, the hydrogel components are cross-linked.

In some embodiments, the one or more co-excipients comprises a polymer selected from Eudragit E PO (EPO); Eudragit RL 100 (RL100); Poly(ethylene glycol) 1 kDa (PEG-1 kDa); Branched Poly(ethyleneimine) 800 Da (PEI:b0.8); Kollidon SR (KOLSR); Poly(methacrylic acid) 5 kDa (PMA:5); Poly(aspartic acid) 2 kDa (POLD:2); Poly(2-ethyloxazoline) 25 kDa (POXZ:25); Poly(glutamic acid) 50 kDa (POLE:50); Branched Poly(ethyleneimine)-co-Poly(ethylene glycol) 500 Da (bPEIPPEG:0.5); Vivacoat (VIVA); and a combination thereof.

In some embodiments, the one or more co-excipients comprises Eudragit E PO (EPO). In some embodiments, the one or more co-excipients comprises Eudragit RL 100 (RL100). In some embodiments, the one or more co-excipients comprises Poly(ethylene glycol) 1 kDa (PEG-1 kDa). In some embodiments, the one or more co-excipients comprises Branched Poly(ethyleneimine) 800 Da (PEI:b0.8). In some embodiments, the one or more co-excipients comprises Kollidon SR (KOLSR). In some embodiments, the one or more co-excipients comprises Poly(methacrylic acid) 5 kDa (PMA:5). In some embodiments, the one or more co-excipients comprises Poly(aspartic acid) 2 kDa (POLD:2). In some embodiments, the one or more co-excipients comprises Poly(2-ethyloxazoline) 25 kDa (POXZ:25). In some embodiments, the one or more co-excipients comprises Poly(glutamic acid) 50 kDa (POLE:50). In some embodiments, the one or more co-excipients comprises Branched Poly(ethyleneimine)-co-Poly(ethylene glycol) 500 Da (bPEIPPEG:0.5). In some embodiments, the one or more co-excipients comprises Vivacoat (VIVA).

### Dendrimers

In some embodiments, the disclosure provides a composition comprising a therapeutic agent, at least one bile acid or salt thereof described herein, and one or more dendrimers.

Dendrimers are repetitively branched polymers comprising a central core surrounded by peripheral groups. Dendritic polymers are typically synthesized stepwise around the core. During the synthesis, each successive reaction step adds a generation of branching. The number of repeated steps corresponds to the dendrimer generation. For example, a single reaction step yields a generation 1 (G1) dendrimer.

In some embodiments, a co-excipient of the disclosure comprises one or more dendrimers. In some embodiments, the dendrimer comprises a poly(amidoamine), poly(propyleneimine), polyamide, polyether, polyester, or phosphorous-based architecture.

In some embodiments, the dendrimer comprises propyleneimine units. In some embodiments, the dendrimer comprises ethylene oxide units. In some embodiments, the dendrimer comprises polybenzyl ether units. In some embodiments, the dendrimer comprises peptidyl units. In some embodiments, the dendrimer comprises saccharide units. In some embodiments, the dendrimer comprises polyester units. In some embodiments, the polymer comprises amidoamine units. In some embodiments, the dendrimer comprises ether units. In some embodiments, the dendrimer comprises polyester units. In some embodiments, the dendrimer is phosphorous based.

In some embodiments, the dendrimer is a G1-G11 dendrimer. In some embodiments, the dendrimer is a G1 dendrimer. In some embodiments, the dendrimer is a G1.5 dendrimer. In some embodiments, the dendrimer is a G2 dendrimer. In some embodiments, the dendrimer is a G2.5 dendrimer. In some embodiments, the dendrimer is a G3 dendrimer. In some embodiments, the dendrimer is a G3.5 dendrimer. In some embodiments, the dendrimer is a G4 dendrimer. In some embodiments, the dendrimer is a G4.5 dendrimer. In some embodiments, the dendrimer is a G5 dendrimer. In some embodiments, the dendrimer is a G5.5 dendrimer. In some embodiments, the dendrimer is a G6 dendrimer. In some embodiments, the dendrimer is a G6.5 dendrimer. In some embodiments, the dendrimer is a G7 dendrimer. In some embodiments, the dendrimer is a G7.5 dendrimer. In some embodiments, the dendrimer is a G8 dendrimer. In some embodiments, the dendrimer is a G8.5 dendrimer. In some embodiments, the dendrimer is a G9.5 dendrimer. In some embodiments, the dendrimer is a G10 dendrimer.

In some embodiments, the dendrimer comprises terminal carboxylates. In some embodiments, the dendrimer comprises terminal amines. In some embodiments, the dendrimer comprises terminal malonate. In some embodiments, the dendrimer comprises terminal hydroxyl groups. In some embodiments, the dendrimer comprises terminal amino groups. In some embodiments, the dendrimer comprises terminal aliphatic groups (e.g., a C2-C20 branched or unbranched alkyl chain).

In some embodiments, the dendrimer is a poly(amidoamine) (PAMAM) dendrimer. In some embodiments, the PAMAM dendrimer is G1-G10. In some embodiments, the PAMAM dendrimer is G1, G1.5, G2, G2.5, G3, G3.5, G4, G4.5, G5, G5.5, G6, G6.5, G7, G7.5, G8, G8.5, G9, G9.5 or G10. In some embodiments, the PAMAM dendrimer comprises terminal carboxylates (carboxylate terminal PAMAM). In some embodiments, the PAMAM dendrimer comprises terminal amines (amine terminal PAMAM). In some embodiments, the PAMAM dendrimer comprises terminal hydroxyl groups (PAMAM-OH). In some embodiments, the PAMAM dendrimer comprises terminal amino groups (PAMAM-NH₂).

In some embodiments, the dendrimer is generation 3.5 PAMAM dendrimer. In some embodiments, the dendrimer is a carboxylate terminal poly(amidoamine) dendrimer generation 3.5 (DENDAC3.5). In some embodiments, the dendrimer is a generation 4 PAMAM dendrimer. In some embodiments, the dendrimer is a amine terminal poly(amidoamine) dendrimer generation 4 (DENDAM4),

### Microscopic Particles

In some embodiments, the disclosure provides a composition comprising a therapeutic agent, at least one bile acid or salt thereof described herein, and one or more microscopic particles. In some embodiments, the microscopic particle comprises nanoparticles, microparticles, or a combination thereof.

As used herein, the term “microparticles” refers to microscopic particles having dimensions on average of less than about 1000 microns. In some embodiments, the microparticle comprises a microsphere. As used herein, the term “microspheres” refers to substantially spherical colloidal structures having a size of at least about 0.5 micron and up to about 1000 microns. In some embodiments, the size is the number average particle size as measured using any method of particle analysis known in the art. In some embodiments, the size is the volume average particle diameter as measured using any method of particle analysis known in the art. Methods of particle analysis include, but are not limited to, light microscopy, scanning electron microscopy, transmission electron microscopy, laser diffraction, light scattering or time of flight.

In some embodiments, the particle (e.g., nanoparticle, microparticle, or combination thereof) is a microcapsule. As used herein, the term “microcapsules” refer to particles (e.g., nanoparticle, microparticle, or combination thereof) having an outer shell surrounding a core of another material. The core can be gas, liquid, gel, solid, or a combination thereof.

As used herein, the term “nanoparticle” refers to microscopic particles having dimensions on average of less than about 1000 nanometers. In some embodiments, the nanoparticle is a substantially spherical particle having an average diameter of about 500, 200, 100, 50 or 10 nm.

In some embodiments, the particle (e.g., nanoparticle, microparticle, or combination thereof) is solid. In some embodiments, the particle (e.g., nanoparticle, microparticle, or combination thereof) is porous. In some embodiments, the particle (e.g., nanoparticle, microparticle, or combination thereof) comprises pores or voids embedded in a matrix material or shell. In some embodiments, the particle (e.g., nanoparticle, microparticle, or combination thereof) comprises the therapeutic agent. In some embodiments, the particle (e.g., nanoparticle, microparticle, or combination thereof) comprises a core comprising the therapeutic agent, wherein the core is encapsulated in a shell or polymeric matrix. In some embodiments, the therapeutic agent is interspersed within the shell or polymeric matrix. In some embodiments, the therapeutic agent is uniformly mixed with the material forming the shell or polymeric matrix.

In some embodiments, the particle (e.g., nanoparticle, microparticle, or combination thereof) is spherical. In some embodiments, the particle (e.g., nanoparticle, microparticle, or combination thereof) is non-spherical, e.g., rod like, sphere like, acieular (slender, needle-like particle of similar width and thickness), columnar (long, thin particle with a width and thickness that are greater than those of an acicular particle), flake (thin, flat particle of similar length and width), plate (flat particle of similar length and width but with greater thickness than flakes), lath (long, thin, blade-like particle), equant (particles of similar length, width, and thickness, this includes both cubical and spherical particles), lamellar (stacked plates), or disc like.

In some embodiments, the particle (e.g., nanoparticle, microparticle, or combination thereof) comprises one or more polymers. In some embodiments, the particle (e.g., nanoparticle, microparticle, or combination thereof) comprises one or more polymers described herein (e.g., poly(hydroxy acids) including polylactic acid, polyglycolic acid, and copolymers thereof, polyanhydrides, polyorthoesters, and certain types of protein and polysaccharide polymers). In some embodiments, the particle (e.g., nanoparticle, microparticle, or combination thereof) is biodegradable.

Suitable methods for making the micro- or nanoparticles include spray-drying, other spray-type manufacturing processes, and solvent removal. One method of making the formulation is described in more detail in WO 2004/098570. Other methods are described in U.S. Pat. No. 6,143,211 to Mathiowitz et al.

In some embodiments, the particle comprises sodium bicarbonate. In some embodiments, the particle comprises calcium phosphate (e.g., calcium phosphate tribasic or calcium phosphate dibasic). In some embodiments, the particle comprises hydroxyapatite. In some embodiments, the particle comprises zinc oxide.

In some embodiments, the diameter of the particle (e.g., the longest diameter if the particle is non-spherical) is about 1 micron, about 2 microns, about 3 microns, about 4 microns, about 5 microns, about 6 microns, about 7 microns, about 8 microns, about 9 microns, about 10 microns, about 15 microns, about 20 microns, about 25 microns, about 30 microns, about 35 microns, about 35 microns, about 40 microns, about 45 microns, or about 50 microns in length.

In some embodiments, the diameter of the particle (e.g., the longest diameter if the particle is non-spherical) is about 50 nanometers, about 100 nanometers, about 150 nanometers, about 200 nanometers, about 250 nanometers, about 300 nanometers, about 350 nanometers, about 400 nanometers, about 450 nanometers, or about 500 nanometers in length.

### Dose and Dosage Form

In some embodiments, a composition of the disclosure comprising one or more doses of a therapeutic agent described herein, an amount of at least one bile acid or salt thereof described herein, and optionally one or more co-excipients described herein, is formulated as a dosage form for in vivo delivery to a subject (e.g., a human subject).

In some embodiments, the disclosure provides a dosage form comprising a composition comprising a therapeutic agent described herein and at least one bile acid or salt thereof described herein. In some embodiments, the disclosure provides a dosage form comprising a composition comprising a therapeutic agent described herein, at least one bile acid or salt thereof described herein, and one or more co-excipients described herein.

In some embodiments, the dosage form is for enteral administration. In some embodiments, the composition is formulated as a dosage form for enteral administration. In some embodiments, the dosage form is for oral administration. In some embodiments, the composition is formulated as a dosage form for oral administration. In some embodiments, the composition is formulated as a dosage form effective for distribution of a substantial portion (e.g., at least about 60%, about 65%, about 70%, about 75%, about 80%, about 85%, about 90%, about 95%, about 98%, about 99%, or about 100%) of the one or more doses of the therapeutic agent to a site of absorption, e.g., following enteral administration to a subject. In some embodiments, the site of absorption comprises one or more sections of the GI tract (e.g., the upper GI tract). In some embodiments, the site of absorption comprises the stomach. In some embodiments, the site of absorption comprises the small intestine. In some embodiments, the site of absorption comprises one or a combination of the duodenum, jejunum, and ileum. In some embodiments, the site of absorption comprises the duodenum and/or the jejunum.

It will be understood that a composition described herein is converted into a dosage form for delivery (e.g., via enteral administration) to a subject using procedures routinely employed by those of skill in the art. For example, in some embodiments, a composition of the disclosure comprises the therapeutic agent in a dosage form that is administered and dosed in accordance with good medical practice, taking into account the clinical condition of the individual patient, the site and method of administration, scheduling of administration, and other factors known to medical practitioners. In human therapy, it is important to provide a dosage form that delivers the effective amount of the drug in vivo, and renders the drug bioavailable in a timely manner.

In some embodiments, the composition is formulated as a solid dosage form. In some embodiments, the solid dosage form is effective for distribution of a substantial portion (e.g., at least about 60%, about 65%, about 70%, about 75%, about 80%, about 85%, about 90%, about 95%, about 98%, about 99%, or about 100%) of the one or more doses of the therapeutic agent to a site of absorption, e.g., following enteral administration to a subject. In some embodiments, the site of absorption is the stomach. In some embodiments, the site of embodiments is the small intestine. In some embodiments, the solid dosage form is effective for releasing a substantial portion (e.g., at least about 60%, about 65%, about 70%, about 75%, about 80%, about 85%, about 90%, about 95%, about 98%, about 99%, or about 100%) of the one or more doses of the therapeutic agent to the liquid volume of the stomach. In some embodiments, the solid dosage form is effective for releasing a substantial portion (e.g., at least about 60%, about 65%, about 70%, about 75%, about 80%, about 85%, about 90%, about 95%, about 98%, about 99%, or about 100%) of the one or more doses of the therapeutic agent to the liquid volume of the small intestine. In some embodiments, the solid dosage form is effective for releasing a substantial portion (e.g., at least about 60%, about 65%, about 70%, about 75%, about 80%, about 85%, about 90%, about 95%, about 98%, about 99%, or about 100%) of the one or more doses of the therapeutic agent to the liquid volume of the stomach and the small intestine combined. In some embodiments, the solid dosage form comprises an amount of the at least one bile acid or salt thereof that when diluted in the liquid volume of the stomach, the liquid volume of the small intestine, or both combined, achieves an effective concentration of the at least one bile acid or salt thereof at the site of absorption shown to enhance GI permeation according to any assay described herein (e.g., GI-ORIS). In some embodiments, the amount of the at least one bile acid or salt thereof is substantially equivalent to the effective concentration multiplied by a dilution factor, wherein the dilution factor is substantially equivalent to the liquid volume of the GI tract or a portion thereof (e.g., the liquid volume of the stomach, the liquid volume of the small intestine, or both combined).

In some embodiments, the composition is formulated as a liquid dosage form. In some embodiments, the liquid dosage form is effective for distribution of a substantial portion (e.g., at least about 60%, about 65%, about 70%, about 75%, about 80%, about 85%, about 90%, about 95%, about 98%, about 99%, or about 100%) of the one or more doses of the therapeutic agent to a site of absorption, e.g., following enteral administration to a subject.

In some embodiments, a dosage form comprising a composition of the disclosure comprises one or more doses of a therapeutic agent described herein. In some embodiments, the one or more doses of the therapeutic agent is an effective amount of the therapeutic agent. As used herein, an “effective amount” refers to an amount of the therapeutic agent that elicits a response for treating an existing disease or disorder and/or preventing or delaying the onset of a disease or disorder in a subject (e.g., a human subject). In some embodiments, the therapeutic effective amount is provided in a single dose. In some embodiments, the therapeutic effective amount is distributed across multiple doses. As understood by the skilled artisan, studies in animal models are generally used for guidance regarding a therapeutical effective amount for treatment of disorders or diseases in a subject. In terms of treatment protocols, it should be appreciated that the dosage to be administered will depend on several factors, including the particular therapeutic agent that is administered, the route chosen for administration, the age of the subject, and the condition of the particular subject. In some embodiments, a dose of the therapeutic agent is selected from a range of about 0.5 g to about 1000 mg.

In some embodiments, the composition is formulated as a dosage form for enteral delivery (e.g., oral, duodenal, or rectal delivery), wherein the composition comprises an amount of at least one bile acid or salts thereof described herein. In some embodiments, the amount is sufficient to provide an effective concentration of the at least one bile acid or salt thereof at the site of absorption (e.g., one or more regions of the GI tract). In some embodiments, the effective concentration is greater than the CMC of the at least one bile acid or salt thereof. In some embodiments, the effective concentration is at least about 2-fold, about 3-fold, about 4-fold, about 5-fold, about 6-fold, about 7-fold, about 8-fold, about 9-fold, or about 10-fold greater than the CMC of the at least one bile acid or salt thereof. In some embodiments, the effective concentration is about 2-fold to about 10-fold, about 2-fold to about 20-fold, about 2-fold to about 30-fold, about 2-fold to about 40-fold, about 2-fold to about 50-fold, about 2-fold to about 100-fold, about 2-fold to about 150-fold, about 2-fold to about 200-fold, 3-fold to about 10-fold, about 3-fold to about 20-fold, about 3-fold to about 30-fold, about 3-fold to about 40-fold, about 3-fold to about 50-fold, about 3-fold to about 100-fold, about 3-fold to about 150-fold, about 3-fold to about 200-fold greater, 3-fold to about 10-fold, about 3-fold to about 20-fold, about 3-fold to about 30-fold, about 3-fold to about 40-fold, about 3-fold to about 50-fold, about 3-fold to about 100-fold, about 3-fold to about 150-fold, about 3-fold to about 200-fold, 4-fold to about 10-fold, about 4-fold to about 20-fold, about 4-fold to about 30-fold, about 4-fold to about 40-fold, about 4-fold to about 50-fold, about 4-fold to about 100-fold, about 4-fold to about 150-fold, or about 4-fold to about 200-fold greater than the CMC of the at least one bile acid or salt thereof. In some embodiments, the effective concentration is about 2-fold, about 3-fold, about 4-fold, about 5-fold, about 10-fold, about 20-fold, about 30-fold, about 40-fold, about 50-fold, about 60-fold, about 70-fold, about 80-fold, about 90-fold, about 100-fold, about 150-fold, or about 200-fold greater than the CMC of the at least one bile acid or salt thereof.

In some embodiments, the amount of the of at least one bile acid or salt thereof is selected to provide an effective concentration of the at least one bile acid or salt thereof at the site of absorption (e.g., one or more regions of the GI tract), wherein the effective concentration is determined using an assay of GI permeation assay described herein (e.g., a permeation assay using a GI-ORIS). In some embodiments, the effective concentration is a concentration of the at least one bile acid or salt thereof that results in increased GI (e.g., intestinal and/or stomach) permeation of the therapeutic agent compared to a control composition comprising the therapeutic agent (e.g., a composition comprising the therapeutic agent without the at least one bile acid or salt thereof) as measured using a GI permeation assay described herein (e.g., a permeation assay using a GI-ORIS).

In some embodiments, the effective concentration (e.g., as measured using an intestinal permeation assay described herein) is at least about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, the effective concentration (e.g., as measured using an intestinal permeation assay described herein) is about 10 mg/mL to about 20 mg/mL, about 10 mg/mL to about 30 mg/mL, about 10 mg/mL to about 40 mg/mL, about 10 mg/mL to about 50 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, about 30 mg/mL to about 100 mg/mL, about 30 mg/mL to about 110 mg/mL, about 30 mg/mL to about 120 mg/mL, about 30 mg/mL to about 130 mg/mL, about 30 mg/mL to about 170 mg/mL, about 30 mg/mL to about 180 mg/mL, about 40 mg/mL to about 160 mg/mL, about 40 mg/mL to about 170 mg/mL, about 40 mg/mL to about 180 mg/mL, about 40 mg/mL to about 190 mg/mL, about 40 mg/mL to about 200 mg/mL, about 50 mg/mL to about 150 mg/mL, about 50 mg/mL to about 160 mg/mL, about 50 mg/mL to about 170 mg/mL, about 50 mg/mL to about 180 mg/mL, about 50 mg/mL to about 190 mg/mL, or about 50 mg/mL to about 200 mg/mL In some embodiments, the effective concentration (e.g., as measured using a permeation assay described herein) is about 30 mg/mL. In some embodiments, the effective concentration (e.g., as measured using a permeation assay described herein) is about 50 mg/mL. In some embodiments, the effective concentration (e.g., as measured using a permeation assay described herein) is about 70 mg/mL. In some embodiments, the effective concentration (e.g., as measured using a permeation assay described herein) is about 90 mg/mL. In some embodiments, the effective concentration (e.g., as measured using a permeation assay described herein) is about 110 mg/mL. In some embodiments, the effective concentration (e.g., as measured using a permeation assay described herein) is about 130 mg/mL. In some embodiments, the effective concentration (e.g., as measured using a permeation assay described herein) is about 150 mg/mL. In some embodiments, the effective concentration (e.g., as measured using a permeation assay described herein) is about 175 mg/mL. In some embodiments, the effective concentration (e.g., as measured using a permeation assay described herein) is about 200 mg/mL.

In some embodiments, the amount of the at least one bile acid or salt thereof is substantially equivalent to the effective concentration (e.g., as a percent weight per volume (e.g., ml/mL)), multiplied by a dilution factor. In some embodiments, the amount of the at least one bile acid or salt thereof is substantially equivalent to the effective concentration, as a percent weight per volume (e.g., ml/mL), multiplied by a dilution factor. As used herein, the term “substantially equivalent” denotes a value that is at least 90%, at least 95%, at least 96%, at least 97%, at least 98% or at least 99% of the reference value.

In some embodiments, the dilution factor is about 1%, 5%, 10%, 15%, 20%, 30%, 40%, 50%, 60%, 70%, 80%, 90%, or 100% of the liquid volume of the GI tract (e.g., the liquid volume of the human GI tract). In some embodiments, the dilution factor is about 1%, 5%, 10%, 15%, 20%, 30%, 40%, 50%, 60%, 70%, 80%, 90%, or 100% of the liquid volume of the stomach (e.g., the liquid volume of the human stomach). In some embodiments, the dilution factor is about 1%, 5%, 10%, 15%, 20%, 30%, 40%, 50%, 60%, 70%, 80%, 90%, or 100% of the liquid volume of the small intestine (e.g., the liquid volume of the human small intestine). In some embodiments, the dilution factor is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, or 10-fold greater or less than the liquid volume of the GI tract (e.g., the liquid volume of the human GI tract). In some embodiments, the percent weight per volume is mg/mL and the dilution factor is about 2 mL to about 30 mL. In some embodiments, the dilution factor is about 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, or 30 mL. In some embodiments, the composition is formulated for immediate release, wherein the dilution factor is less than about 30 mL, less than about 25 mL, less than about 20 mL, or less than about 15 mL. In some embodiments, the composition is formulated for immediate release, wherein the dilution factor is about 10 mL to about 20 mL. In some embodiments, the composition is formulated for immediate release, wherein the dilution factor is about 5 mL to about 15 mL. In some embodiments, the composition is formulated for delayed release, wherein the dilution factor is less than about 15 mL, less than about 10 mL, or less than about 5 mL. In some embodiments, the composition is formulated for immediate release, wherein the dilution factor is about 2 mL to about 15 mL. In some embodiments, the composition is formulated for immediate release, wherein the dilution factor is about 2 mL to about 10 mL.

In some embodiments, the amount of the at least one bile acid or salt thereof is about 60 mg to about 2,000 mg. In some embodiments, the amount of the at least one bile acid or salt thereof is about 60 mg to about 1,000 mg. In some embodiments, the amount of the at least one bile acid or salt thereof is about 60 mg to about 600 mg. In some embodiments, the amount of the least one bile acid or salt thereof is about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the composition comprises an amount of one bile acid or salt thereof selected to provide an effective concentration of the one bile acid or salt thereof at the site of absorption (e.g., one or more regions of the GI tract), wherein the effective concentration is determined using an assay of GI permeation assay described herein (e.g., a permeation assay using a GI-ORIS). In some embodiments, the amount of the one bile acid or salt thereof is substantially equivalent to the effective concentration (e.g., as a percent weight per volume (e.g., ml/mL)), multiplied by a dilution factor described herein (e.g., about 2 mL to about 30 mL). In some embodiments, the amount of the one bile acid or salt thereof is about 60 mg to about 2,000 mg. In some embodiments, the amount of the one bile acid or salt thereof is about 60 mg to about 1,000 mg. In some embodiments, the amount of the one bile acid or salt thereof is about 60 mg to about 600 mg. In some embodiments, the amount of the one bile acid or salt thereof is about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the composition comprises an amount of two or more bile acids or salts thereof (e.g., 2, 3, 4, 5, 6, 7, 8, 9, or 10 bile acids or salts thereof), wherein the amount is selected to provide an effective concentration of the two or more bile acids or salts thereof at the site of absorption (e.g., one or more regions of the GI tract), wherein the effective concentration is determined using an assay of GI permeation assay described herein (e.g., a permeation assay using a GI-ORIS). In some embodiments, the amount of the two or more bile acids or salts thereof is substantially equivalent to the effective concentration (e.g., as a percent weight per volume (e.g., ml/mL)), multiplied by a dilution factor described herein (e.g., about 2 mL to about 30 mL). In some embodiments, the amount of the two or more bile acid or salt thereof is each independently about 60 mg to about 2,000 mg. In some embodiments, the amount of the two or more bile acid or salt thereof is each independently about 60 mg to about 1,000 mg. In some embodiments, the amount of the two or more bile acid or salt thereof is each independently about 60 mg to about 600 mg. In some embodiments, the amount of the two or more bile acid or salt thereof is each independently about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the at least two or more bile acids or salts thereof comprises a first bile acid or salt thereof and a second bile acid or salt thereof, wherein the molar ratio of the first bile acid or salt thereof to the second bile acid or salt thereof is about 1:1, about 1.5:1, about 2:1, about 2.5:1, about 3:1, about 3.5:1, about 4:1, about 4.5:1, about 5:1, about 6:1, about 7:1, about 8:1, about 9:1, or about 10:1. In some embodiments, the at least two or more bile acids or salts thereof comprises a first bile acid or salt thereof, a second bile acid or salt thereof, and a third bile acid or salt thereof, wherein the composition comprises an equimolar amount of the first, second, and third bile acid or salt thereof. In some embodiments, the at least two or more bile acids or salts thereof comprises a first bile acid or salt thereof, a second bile acid or salt thereof, a third bile acid or salt thereof, and a fourth bile acid or salt thereof, wherein the composition comprises an equimolar amount of the first, second, third, and fourth bile acid or salt thereof. In some embodiments, the dosage form comprises a composition described herein comprising an amount of a therapeutic agent described herein and an amount of at least one bile acid or salt thereof, wherein the composition comprises a molar ratio of the amount of the at least one bile acid or salt thereof to an amount of the therapeutic agent that is about 10:1, about 20:1, about 30:1, about 40:1, about 50:1, about 100:1, about 150:1, about 200:1, about 300:1, about 400:1, or about 500:1.

In some embodiments, the composition is formulated as a dosage form for enteral delivery (e.g., oral, duodenal, or rectal delivery), wherein the composition comprises an amount of one or more co-excipients selected from a chelator, a polymer, a dendrimer, a nanoparticle, a lipid, an alkyl acid, and a combination thereof. In some embodiments, the amount is sufficient to provide an effective concentration of the one or more co-excipients at the site of absorption (e.g., one or more regions of the GI tract). In some embodiments, the effective concentration is determined using an assay of GI permeation assay described herein (e.g., a permeation assay using a GI-ORIS). In some embodiments, the effective concentration is a concentration of the one or more co-excipients that results in increased GI permeation of the therapeutic agent compared to a control composition comprising the therapeutic agent (e.g., a composition comprising the therapeutic agent without the at least one bile acid or salt thereof and the one or more co-excipients) as measured using an GI permeation assay described herein (e.g., a permeation assay using a GI-ORIS). In some embodiments, the effective concentration is a concentration of the one or more co-excipients that results in increased intestinal permeation of the therapeutic agent compared to a control composition comprising the therapeutic agent (e.g., a composition comprising the therapeutic agent without the at least one bile acid or salt thereof and the one or more co-excipients) as measured using a GI permeation assay described herein (e.g., a permeation assay using a GI-ORIS). In some embodiments, the amount of the one or more co-excipients is substantially equivalent to the effective concentration (e.g., as a percent weight per volume (e.g., ml/mL)), multiplied by a dilution factor described herein (e.g., about 2 mL to about 30 mL).

In some embodiments, the amount of the one or more co-excipients is about 20 mg to about 2,000 mg. In some embodiments, the amount of the one or more co-excipients is about 20 mg to about 1,000 mg. In some embodiments, the amount of the one or more co-excipients is about 20 mg to about 600 mg. In some embodiments, the amount of one or more co-excipients is about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the composition is formulated as a dosage form for enteral delivery (e.g., oral, duodenal, or rectal delivery), wherein the composition comprises an amount of one or more chelators described herein (e.g., ethyleneglycol-bis(P-aminoethyl)-N,N,N′,N′-tetraacetic acid or salt thereof, ethylenedinitrilotetraacetic acid or a salt thereof, glutamate or a salt thereof). In some embodiments, the amount is sufficient to provide an effective concentration of the one or more chelators at the site of absorption (e.g., one or more regions of the GI tract). In some embodiments, the effective concentration is a concentration of the one or more chelators that results in increased GI permeation of the therapeutic agent compared to a control composition comprising the therapeutic agent (e.g., a composition comprising the therapeutic agent without the at least one bile acid or salt thereof and the one or more chelators) as measured using an GI permeation assay described herein (e.g., a permeation assay using a G1-ORIS). In some embodiments, the effective concentration is a concentration of the one or more chelators that results in increased intestinal permeation of the therapeutic agent compared to a control composition comprising the therapeutic agent (e.g., a composition comprising the therapeutic agent without the at least one bile acid or salt thereof and the one or more chelators) as measured using an GI permeation assay described herein (e.g., a permeation assay using a G1-ORIS). In some embodiments, the effective concentration of the one or more chelators (e.g., measured using a GI permeation assay described herein, e.g., GI-ORIS) is at least about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL.

In some embodiments, the effective concentration of the one or more chelators (e.g., measured using a GI permeation assay described herein, e.g., GI-ORIS) is about 5 mg/mL to about 20 mg/mL, about 5 mg/mL to about 30 mg/mL, about 10 mg/mL to about 40 mg/mL, about 10 mg/mL to about 50 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, about 30 mg/mL to about 100 mg/mL, about 30 mg/mL to about 110 mg/mL, about 30 mg/mL to about 120 mg/mL, about 30 mg/mL to about 130 mg/mL, about 30 mg/mL to about 170 mg/mL, about 30 mg/mL to about 180 mg/mL, or about 40 mg/mL to about 150 mg/mL. In some embodiments, the amount of the one or more chelators is substantially equivalent to the effective concentration (e.g., as a percent weight per volume (e.g., ml/mL)), multiplied by a dilution factor described herein (e.g., about 2 mL to about 30 mL). In some embodiments, the amount of the chelator is about 10 mg to about 1,500 mg. In some embodiments, the amount of the chelator is about 10 mg to about 1,000 mg. In some embodiments, the amount of the chelator is about 10 mg to about 500 mg. In some embodiments, the amount of the chelator is about 10 mg to about 250 mg. In some embodiments, the amount of the chelator is about 10 mg to about 100 mg.

In some embodiments, the composition is formulated as a dosage form for enteral delivery (e.g., oral, duodenal, or rectal delivery), wherein the composition comprises an amount of one or more polymers described herein. In some embodiments, the amount is sufficient to provide an effective concentration of the one or more polymers at the site of absorption (e.g., one or more regions of the GI tract). In some embodiments, the effective concentration is a concentration of the one or more polymers that results in increased GI permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., an permeation assay using a GI-ORIS). In some embodiments, the effective concentration is a concentration of the one or more polymers that results in increased intestinal permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., a permeation assay using a GI-ORIS). In some embodiments, the effective concentration of the one or more polymers (e.g., measured using an GI permeation assay described herein, e.g., a permeation assay using a GI-ORIS) is at least about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL. In some embodiments, the effective concentration of the one or more polymers (e.g., measured using an GI permeation assay described herein, e.g., a permeation assay using a GI-ORIS) is about 5 mg/mL to about 20 mg/mL, about 5 mg/mL to about 30 mg/mL, about 10 mg/mL to about 40 mg/mL, about 10 mg/mL to about 50 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, about 30 mg/mL to about 100 mg/mL, about 30 mg/mL to about 110 mg/mL, about 30 mg/mL to about 120 mg/mL, about 30 mg/mL to about 130 mg/mL, about 30 mg/mL to about 170 mg/mL, about 30 mg/mL to about 180 mg/mL, or about 40 mg/mL to about 150 mg/mL. In some embodiments, the amount of the one or more polymers is substantially equivalent to the effective concentration (e.g., as a percent weight per volume (e.g., ml/mL)), multiplied by a dilution factor described herein (e.g., about 2 mL to about 30 mL).

In some embodiments, the amount of the polymer is about 10 mg to about 1,500 mg. In some embodiments, the amount of the polymer is about 10 mg to about 1,000 mg. In some embodiments, the amount of the polymer is about 10 mg to about 500 mg. In some embodiments, the amount of the polymer is about 10 mg to about 250 mg. In some embodiments, the amount of the polymer is about 10 mg to about 100 mg.

In some embodiments, the composition is formulated as a dosage form for enteral delivery (e.g., oral, duodenal, or rectal delivery), wherein the composition comprises an amount of one or more dendrimers described herein (e.g., a G1-G10 PAMAM dendrimer). In some embodiments, the amount is sufficient to provide an effective concentration of the one or more dendrimers at the site of absorption (e.g., one or more regions of the GI tract). In some embodiments, the effective concentration is a concentration of the one or more dendrimers that results in increased GI permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., a permeation assay using a GI-ORIS). In some embodiments, the effective concentration is a concentration of the one or more dendrimers that results in increased intestinal permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., a permeation assay using a GI-ORIS). In some embodiments, the effective concentration of the one or more dendrimers (e.g., measured using a GI permeation assay described herein, e.g., GI-ORIS) is at least about 0.01 mg/mL, about 0.05 mg/mL, about 0.1 mg/mL, about 0.2 mg/mL, about 0.3 mg/mL, about 0.4 mg/mL, about 0.5 mg/mL, about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL. In some embodiments, the effective concentration of the one or more dendrimers (e.g., measured using a GI permeation assay described herein, e.g., a permeation assay using a GI-ORIS) is about 0.01 mg/mL to about 1 mg/mL, about 0.05 mg/mL to about 1 mg/mL, about 0.5 mg/mL to about 2 mg/mL, about 1 mg/mL to about 5 mg/mL, about 1 mg/mL to about 10 mg/mL, about 1 mg/mL to about 15 mg/mL, about 1 mg/mL to about 20 mg/mL, about 1 mg/mL to about 30 mg/mL, about 5 mg/mL to about 20 mg/mL, about 5 mg/mL to about 30 mg/mL, about 10 mg/mL to about 40 mg/mL, about 10 mg/mL to about 50 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, or about 30 mg/mL to about 100 mg/mL. In some embodiments, the amount of the one or more dendrimers is substantially equivalent to the effective concentration (e.g., as a percent weight per volume (e.g., ml/mL)), multiplied by a dilution factor described herein (e.g., about 2 mL to about 30 mL).

In some embodiments, the amount of the one or more dendrimers is about 0.1 mg to about 1,500 mg. In some embodiments, the amount of the one or more dendrimers is about 0.1 mg to about 1,000 mg. In some embodiments, the amount of the one or more dendrimers is about 0.1 mg to about 500 mg. In some embodiments, the amount of the one or more dendrimers is about 0.1 mg to about 250 mg. In some embodiments, the amount of the one or more dendrimers is about 0.1 mg to about 100 mg. In some embodiments, the amount of the one or more dendrimers is about 0.1 mg to about 50 mg. In some embodiments, the amount of the one or more dendrimers is about 0.1 mg to about 20 mg. In some embodiments, the amount of the one or more dendrimers is about 0.1 mg to about 10 mg. In some embodiments, the amount of the one or more dendrimers is about 1 mg to about 10 mg.

In some embodiments, the composition is formulated as a dosage form for enteral delivery (e.g., oral, duodenal, or rectal delivery), wherein the composition comprises an amount of one or more microscopic particles described herein (e.g., a metal oxide, metal salt, mineral colloid, inorganic salt, or carbon-based nanoparticle or microparticle). In some embodiments, the amount is sufficient to provide an effective concentration of the one or more microscopic particles at the site of absorption (e.g., one or more regions of the GI tract). In some embodiments, the effective concentration is a concentration of the one or more microscopic particles that results in increased GI permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., a permeation assay using a GI-ORIS). In some embodiments, the effective concentration is a concentration of the one or more microscopic particles that results in increased intestinal permeation of the therapeutic agent compared to a control composition comprising the therapeutic agent (e.g., a composition comprising the therapeutic agent without the at least one bile acid or salt thereof and the one or more polymers) as measured using a GI permeation assay described herein (e.g., a permeation assay using a G1-ORIS). In some embodiments, the effective concentration of the one or more microscopic particles (e.g., measured using a GI permeation assay described herein, e.g., a permeation assay using a GI-ORIS) is at least about 0.01 mg/mL, about 0.05 mg/mL, about 0.1 mg/mL, about 0.2 mg/mL, about 0.3 mg/mL, about 0.4 mg/mL, about 0.5 mg/mL, about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL. In some embodiments, the effective concentration of the one or more microscopic particles (e.g., measured using an GI permeation assay described herein, e.g., a permeation assay using a GI-ORIS) is about 0.01 mg/mL to about 1 mg/mL, about 0.05 mg/mL to about 1 mg/mL, about 0.5 mg/mL to about 2 mg/mL, about 1 mg/mL to about 5 mg/mL, about 1 mg/mL to about 10 mg/mL, about 1 mg/mL to about 15 mg/mL, about 1 mg/mL to about 20 mg/mL, about 1 mg/mL to about 30 mg/mL, about 5 mg/mL to about 20 mg/mL, about 5 mg/mL to about 30 mg/mL, about 10 mg/mL to about 40 mg/mL, about 10 mg/mL to about 50 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, or about 30 mg/mL to about 100 mg/mL. In some embodiments, the amount of the one or more microscopic particles is substantially equivalent to the effective concentration (e.g., as a percent weight per volume (e.g., ml/mL)), multiplied by a dilution factor described herein (e.g., about 2 mL to about 30 mL).

In some embodiments, the amount of the microscopic particle is about 0.2 mg to about 1,500 mg. In some embodiments, the amount of the microscopic particle is about 0.2 mg to about 1,000 mg. In some embodiments, the amount of the microscopic particle is about 0.2 mg to about 500 mg. In some embodiments, the amount of the microscopic particle is about 0.2 mg to about 250 mg. In some embodiments, the amount of the microscopic particle is about 0.2 mg to about 100 mg. In some embodiments, the amount of the microscopic particle is about 0.2 mg to about 50 mg. In some embodiments, the amount of the microscopic particle is about 0.2 mg to about 20 mg. In some embodiments, the amount of the microscopic particle is about 0.2 mg to about 10 mg. In some embodiments, the amount of the microscopic particle is about 1 mg to about 10 mg.

### Methods to Prepare a Dosage Form

Provided herein are methods of preparing a dosage form comprising a composition of the disclosure. In some embodiments, the method comprises obtaining an amount of each component of the composition (e.g., one or more doses of the therapeutic agent, an amount of the at least one bile acid or salt thereof, and optionally an amount of the one or more co-excipients) and formulating the amounts into a dosage form described herein (e.g., a solid dosage form, a semi-solid dosage form, or a liquid dosage form). Methods and processes to formulate a dosage form describe herein known in the art.

In some embodiments, the formulation into a liquid dosage form comprises combining the amounts of the components of the composition with a liquid vehicle (e.g., an aqueous solvent or buffer, an alcohol solvent, glycerin, propylene glycol, an oil, or a combination thereof), surfactant, co-solvent, preservative, viscosity modifier, or suspending agent, such as any described herein. In some embodiments, the formulation comprises preparation of a syrup (a viscous liquid comprising the components of the composition in solution), a suspension (a liquid comprising one or more components of the composition in suspension in a liquid carrier), a solution (a liquid comprising the components of the composition solubilized in a liquid carrier), a drop (a liquid prepared in small quantity for administration using a measuring device such as a dropper), an emulsion (an oil in water dispersion comprising components of the composition in one or both phases of the mixture), a mixture (a liquid comprising one or more components of the composition suspended or dispersed in a liquid carrier), a linctus (a viscous liquid comprising one or more components of the composition dissolved in a liquid carrier comprising a high concentration of sucrose or other sugar), or an elixir (a liquid comprising one or more components of the composition dissolved in a carrier comprising a high proportion of sucrose and optionally an alcohol).

In some embodiments, the formulation into a solid dosage form comprises powder grinding, dry powder mixing, and tableting via direct compression. In some embodiments, the formulation into a solid dosage form comprises a wet granulation process. Such a method (such as high shear granulation process) comprises mixing the components of the composition and optionally, the one or combination of factors, in a mixer. The binder is added in the mix dry or dissolved in a fluid used for granulation. The granulating solution or suspension is added to the dry powders in the mixer and mixed until the desired characteristics are achieved. This typically produces granules of suitable characteristics for producing dosage forms with adequate dissolution time, content uniformity, and other physical characteristics. After the wet granulation step, the product is dried and/or then milled after drying to get a major percentage of the product within a desired size range. As is understood by the skilled artisan, the product may be dried after being wet-sized using a suitable device, such as an oscillating granulator or a mill. The dry granulation mix may then be processed to get an acceptable size range by first screening with a sieving device, and then milling the oversized particles. In some embodiments, an appropriate glidant is added to improve the flow properties of the granules.

In some embodiments, the formulation into a solid dosage form comprises an alternative granulation process known in the art, e.g., spray fluid bed granulation, extrusion and spheronization, or fluid bed rotor granulation.

In some embodiments, the solid dosage form comprises a coating. In some embodiments, the coating is selected to protect the core against damage (e.g., resulting from transport and storage) and/or for aesthetic purposes (e.g., by providing a desired color and/or taste). In some embodiments, the coating is performed by a method known in the art, e.g., W\ddot{ν}urster coating, dry coating, film coating, fluid bed coating, pan coating, etc. In some embodiments, the coating material is selected from polyvinyl pyrrolidone (PVP), polyvinyl pyrrolidone vinyl acetate copolymer (PVPVA), polyvinyl alcohol (PVA), polyvinyl alcohol/polyethylene glycol copolymer (PV A/PEG), cellulose acetate phthalate, ethyl cellulose, gellan gum, maltodextrin, methacrylates, methyl cellulose, hydroxyl propyl methyl cellulose, and carraeeenan.

In some embodiments, the coating comprises a bioadhesive material. In some embodiments, the coating improves bioadhesion of the tablet in the sublingual cavity. In some embodiments, the solid dosage form is prepared for rapid disintegration, wherein the solid dosage form is a tablet comprising an eroding or hydrogel core coated with a bioadhesive material, wherein when the solid dosage form is contacted with saliva, the hydrogel core swells and exerts force on the coat, resulting in its breakdown and fast erosion. In some embodiments, the core comprises a disintegrants to facilitate the erosion.

In some embodiments, the coating comprises a moisture-resistant material (e.g., a hydrophobic polymer) to create a barrier for moisture ingress, thereby protecting a moisture-sensitive therapeutic agent present in the table core (e.g., during storage and transport). A number of coating materials can be used to improve the moisture resistance of the tablet such as EUDRAGIT® E PO, Opadry® AMB5 starch acetate and the like.

### Exemplary Formulations

The present disclosure provides compositions for enhancing delivery of a therapeutic agent to the GI tract. In some embodiments, the composition is formulated as a dosage form for enteral delivery (e.g., oral, duodenal, or rectal delivery). In some embodiments, the composition is formulated as a dosage form for oral delivery. In some embodiments, the composition is effective for increasing GI permeation of the therapeutic agent relative to GI permeation of a control composition comprising the therapeutic agent alone. In some embodiments, the GI permeation is measured using a permeation assay described herein, e.g., a permeation assay using a GI-ORIS.

In some embodiments, the composition comprises one or more doses of a therapeutic agent, and an amount of at least one bile acid or salt thereof at a concentration greater than the CMC of the bile acid or salt thereof. In some embodiments, the composition comprises one or more doses of a therapeutic agent, an amount of at least one bile acid or salt thereof at a concentration greater than the CMC of the bile acid or salt thereof, and an amount of one or more co-excipients described herein. In some embodiments, the composition comprises one or more doses of a therapeutic agent, an amount of at least one bile acid or salt thereof at a concentration greater than the CMC of the bile acid or salt thereof, and an amount of one or more co-excipients described herein. In some embodiments, the one or more co-excipients are selected from a chelator, a polymer, a microparticle, a nanoparticle, a dendrimer, and a combination thereof.

In some embodiments, the composition comprises one or more doses of a therapeutic agent, and an amount of a compound of Formula (I) described herein. In some embodiments, the composition comprises one or more doses of a therapeutic agent, and an amount of at least one bile acid or salt thereof at a concentration greater than the CMC of the bile acid or salt thereof, wherein the at least one bile acid or salt thereof is a compound of Formula (I) described herein.

In some embodiments, the composition comprises one or more doses of a therapeutic agent, an amount of a compound of Formula (I) described herein, and an amount of one or more co-excipients described herein. In some embodiments, the composition comprises one or more doses of a therapeutic agent, an amount of at least one bile acid or salt thereof at a concentration greater than the CMC of the bile acid or salt thereof, wherein the at least one bile acid or salt thereof is a compound of Formula (I) described herein, and an amount of one or more co-excipients described herein.

In some embodiments, the composition comprises one or more doses of a therapeutic agent, and an amount of at least one bile acid or salt thereof, wherein the at least one bile acid or salt thereof is a compound selected from Table A. In some embodiments, the composition comprises one or more doses of a therapeutic agent, and an amount of at least one bile acid or salt thereof at a concentration greater than the CMC of the bile acid or salt thereof, wherein the at least one bile acid or salt thereof is a compound selected from Table A.

In some embodiments, the composition comprises one or more doses of a therapeutic agent, an amount of at least one bile acid or salt thereof, wherein the at least one bile acid or salt thereof is a compound selected from Table A, and an amount of one or more co-excipients described herein. In some embodiments, the composition comprises one or more doses of a therapeutic agent, an amount of at least one bile acid or salt thereof at a concentration greater than the CMC of the bile acid or salt thereof, wherein the at least one bile acid or salt thereof is a compound selected from Table A, and an amount of one or more co-excipients described herein. In some embodiments, the composition comprises one or more doses of a therapeutic agent, an amount of at least one bile acid or salt thereof that is substantially equivalent to a concentration (weight per volume) greater than the CMC of the bile acid or salt thereof multiplied by a dilution factor described herein (e.g., about 2 mL to about 30 mL), wherein the at least one bile acid or salt thereof is a compound selected from Table A, and an amount of one or more co-excipients described herein.

In some embodiments, the composition comprises one or more doses of a therapeutic agent, and an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof. In some embodiments, the at least one bile acid. In some embodiments, the composition comprises one or more doses of a therapeutic agent, an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof, and an amount of one or more co-excipients, wherein the one or more co-excipients are selected from a chelator, a polymer, a microparticle, a nanoparticle, a dendrimer, and a combination thereof. In some embodiments, the therapeutic agent is characterized by poor GI permeability. In some embodiments, the therapeutic agent is a class III compound according to the BCS. In some embodiments, the therapeutic agent is a class IV compound according to the BCS.

In some embodiments, the composition comprises one or more doses of a therapeutic agent, wherein the therapeutic agent is a class III or class IV compound according to the BCS, and an amount of at least one bile acid or salt thereof described herein. In some embodiments, the composition comprises one or more doses of a therapeutic agent, wherein the therapeutic agent is a class III or class IV compound according to the BCS, and an amount of at least one bile acid or salt thereof at a concentration greater than the CMC of the bile acid or salt thereof. In some embodiments, the composition comprises one or more doses of a therapeutic agent, wherein the therapeutic agent is a class III or class IV compound according to the BCS, an amount of at least one bile acid or salt thereof, and an amount of one or more co-excipients described herein. In some embodiments, the composition comprises one or more doses of a therapeutic agent, wherein the therapeutic agent is a class III or class IV compound according to the BCS, an amount of at least one bile acid or salt thereof at a concentration greater than the CMC of the bile acid or salt thereof, and an amount of one or more co-excipients described herein. In some embodiments, the composition comprises one or more doses of a therapeutic agent, wherein the therapeutic agent is a class III or class IV compound according to the BCS, an amount of at least one bile acid or salt thereof at a concentration greater than the CMC of the bile acid or salt thereof, and an amount of one or more co-excipients described herein, wherein the one or more co-excipients are selected from a chelator, a polymer, a microparticle, a nanoparticle, a dendrimer, and a combination thereof.

In some embodiments, the composition comprises one or more doses of a therapeutic agent, wherein the therapeutic agent is a class III or class IV compound according to the BCS, and an amount of a compound of Formula (I) described herein. In some embodiments, the composition comprises one or more doses of a therapeutic agent, wherein the therapeutic agent is a class III or class IV compound according to the BCS, and an amount of at least one bile acid or salt thereof at a concentration greater than the CMC of the bile acid or salt thereof, wherein the at least one bile acid or salt thereof is a compound of Formula (I) described herein.

In some embodiments, the composition comprises one or more doses of a therapeutic agent, wherein the therapeutic agent is a class III or class IV compound according to the BCS, an amount of a compound of Formula (I) described herein, and an amount of one or more co-excipients described herein. In some embodiments, the composition comprises one or more doses of a therapeutic agent, wherein the therapeutic agent is a class III or class IV compound according to the BCS, an amount of at least one bile acid or salt thereof at a concentration greater than the CMC of the bile acid or salt thereof, wherein the at least one bile acid or salt thereof is a compound of Formula (I) described herein, and an amount of one or more co-excipients described herein.

In some embodiments, the composition comprises one or more doses of a therapeutic agent, wherein the therapeutic agent is a class III or class IV compound according to the BCS, and an amount of at least one bile acid or salt thereof, wherein the at least one bile acid or salt thereof is a compound selected from Table A. In some embodiments, the composition comprises one or more doses of a therapeutic agent, wherein the therapeutic agent is a class III or class IV compound according to the BCS, and an amount of at least one bile acid or salt thereof at a concentration greater than the CMC of the bile acid or salt thereof, wherein the at least one bile acid or salt thereof is a compound selected from Table A.

In some embodiments, the composition comprises one or more doses of a therapeutic agent, wherein the therapeutic agent is a class III or class IV compound according to the BCS, an amount of at least one bile acid or salt thereof, wherein the at least one bile acid or salt thereof is a compound selected from Table A, and an amount of one or more co-excipients described herein. In some embodiments, the composition comprises one or more doses of a therapeutic agent, wherein the therapeutic agent is a class III or class IV compound according to the BCS, an amount of at least one bile acid or salt thereof at a concentration greater than the CMC of the bile acid or salt thereof, wherein the at least one bile acid or salt thereof is a compound selected from Table A, and an amount of one or more co-excipients described herein.

In some embodiments, composition comprises one or more doses of a therapeutic agent, wherein the therapeutic agent is a class III or class IV compound according to the BCS, and an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof. In some embodiments, composition comprises one or more doses of a therapeutic agent, wherein the therapeutic agent is a class III or class IV compound according to the BCS, an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof, and an amount of one or more co-excipients, wherein the one or more-coexcipients is selected from a chelator, a polymer, a microparticle, a nanoparticle, a dendrimer, and a combination thereof.

In some embodiments, the therapeutic agent is selected from a polypeptide, a nucleic acid, an oligosaccharide, a small molecule, a lipid, and a combination thereof. In some embodiments, the therapeutic agent has a molecular weight greater than about 1 kDa, about 2 kDa, about 3 kDa, about 4 kDa, about 5 kDa, about 10 kDa, about 15 kDa, about 20 kDa, about 25 kDa, or about 30 kDa. In some embodiments, the therapeutic agent has a molecular weight of about 0.1 to about 1 kDa.

In some embodiments, the composition comprises one or more doses of a therapeutic agent, wherein the therapeutic agent is a polypeptide having a molecular weight of about 1 kDa to 250 kDa, and an amount of at least one bile acid or salt thereof at a concentration greater than the CMC of the bile acid or salt thereof. In some embodiments, the composition comprises one or more doses of a therapeutic agent, wherein the therapeutic agent is a polypeptide having a molecular weight of about 1 kDa to 250 kDa, an amount of at least one bile acid or salt thereof at a concentration greater than the CMC of the bile acid or salt thereof, and an amount of one or more co-excipients described herein. In some embodiments, the composition comprises one or more doses of a therapeutic agent, wherein the therapeutic agent is a polypeptide having a molecular weight of about 1 kDa to 250 kDa, an amount of at least one bile acid or salt thereof at a concentration greater than the CMC of the bile acid or salt thereof, and an amount of one or more co-excipients described herein, wherein the one or more co-excipients are selected from a chelator, a polymer, a microparticle, a nanoparticle, a dendrimer, and a combination thereof.

In some embodiments, the composition comprises one or more doses of a polypeptide having a molecular weight of about 1 kDa to 250 kDa, and an amount of a compound of Formula (I) described herein. In some embodiments, the composition comprises one or more doses of a polypeptide having a molecular weight of about 1 kDa to 250 kDa, and an amount of at least one bile acid or salt thereof at a concentration greater than the CMC of the bile acid or salt thereof, wherein the at least one bile acid or salt thereof is a compound of Formula (I) described herein.

In some embodiments, the composition comprises one or more doses of a polypeptide having a molecular weight of about 1 kDa to 250 kDa, an amount of a compound of Formula (I) described herein, and an amount of one or more co-excipients described herein. In some embodiments, the composition comprises one or more doses of a polypeptide having a molecular weight of about 1 kDa to 250 kDa, an amount of at least one bile acid or salt thereof at a concentration greater than the CMC of the bile acid or salt thereof, wherein the at least one bile acid or salt thereof is a compound of Formula (I) described herein, and an amount of one or more co-excipients described herein.

In some embodiments, the composition comprises one or more doses of a polypeptide having a molecular weight of about 1 kDa to 250 kDa, and an amount of at least one bile acid or salt thereof, wherein the at least one bile acid or salt thereof is a compound selected from Table A. In some embodiments, the composition comprises one or more doses of a polypeptide having a molecular weight of about 1 kDa to 250 kDa, and an amount of at least one bile acid or salt thereof at a concentration greater than the CMC of the bile acid or salt thereof, wherein the at least one bile acid or salt thereof is a compound selected from Table A.

In some embodiments, the composition comprises one or more doses of a polypeptide having a molecular weight of about 1 kDa to 250 kDa, an amount of at least one bile acid or salt thereof, wherein the at least one bile acid or salt thereof is a compound selected from Table A, and an amount of one or more co-excipients described herein. In some embodiments, the composition comprises one or more doses of a polypeptide having a molecular weight of about 1 kDa to 250 kDa, an amount of at least one bile acid or salt thereof at a concentration greater than the CMC of the bile acid or salt thereof, wherein the at least one bile acid or salt thereof is a compound selected from Table A, and an amount of one or more co-excipients described herein.

In some embodiments, the composition comprises one or more doses of a therapeutic agent, wherein the therapeutic agent is a polypeptide having a molecular weight of about 1 kDa to 250 kDa, and an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof. In some embodiments, the composition comprises one or more doses of a therapeutic agent, wherein the therapeutic agent is a polypeptide having a molecular weight of about 1 kDa to 250 kDa, an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and a combination thereof and an amount of one or more co-excipients described herein, wherein the one or more co-excipients are selected from a chelator, a polymer, a microparticle, a nanoparticle, a dendrimer, and a combination thereof.

In some embodiments, the polypeptide is a class III compound according to the BCS. In some embodiments, the polypeptide is a class IV compound according to BCS. In some embodiments, the polypeptide is selected from an enzyme, an antibody or an antigen binding fragment thereof, an antibacterial agent, a hormone, a growth factor, a chemokine, and a cytokine. In some embodiments, the polypeptide is a recombinant protein. In some embodiments, the polypeptide is a therapeutic peptide.

In some embodiments, the composition comprises one or more doses of a therapeutic agent, wherein the therapeutic agent is a nucleic acid or a vector comprising the nucleic acid, and an amount of at least one bile acid or salt thereof at a concentration greater than the CMC of the bile acid or salt thereof. In some embodiments, the composition comprises one or more doses of a therapeutic agent, wherein the therapeutic agent is a nucleic acid or a vector comprising the nucleic acid, an amount of at least one bile acid or salt thereof at a concentration greater than the CMC of the bile acid or salt thereof, and an amount of one or more co-excipients described herein. In some embodiments, the composition comprises one or more doses of a therapeutic agent, wherein the therapeutic agent is a nucleic acid or a vector comprising the nucleic acid, an amount of at least one bile acid or salt thereof at a concentration greater than the CMC of the bile acid or salt thereof, and an amount of one or more co-excipients described herein, wherein the one or more co-excipients are selected from a chelator, a polymer, a microparticle, a nanoparticle, a dendrimer, and a combination thereof.

In some embodiments, the composition comprises one or more doses of a nucleic acid or a vector comprising the nucleic acid, and an amount of a compound of Formula (I) described herein. In some embodiments, the composition comprises one or more doses of a nucleic acid or a vector comprising the nucleic acid, and an amount of at least one bile acid or salt thereof at a concentration greater than the CMC of the bile acid or salt thereof, wherein the at least one bile acid or salt thereof is a compound of Formula (I) described herein.

In some embodiments, the composition comprises one or more doses of a nucleic acid or a vector comprising the nucleic acid, an amount of a compound of Formula (I) described herein, and an amount of one or more co-excipients described herein. In some embodiments, the composition comprises one or more doses of a nucleic acid or a vector comprising the nucleic acid, an amount of at least one bile acid or salt thereof at a concentration greater than the CMC of the bile acid or salt thereof, wherein the at least one bile acid or salt thereof is a compound of Formula (I) described herein, and an amount of one or more co-excipients described herein.

In some embodiments, the composition comprises one or more doses of a nucleic acid or a vector comprising the nucleic acid, and an amount of at least one bile acid or salt thereof, wherein the at least one bile acid or salt thereof is a compound selected from Table A. In some embodiments, the composition comprises one or more doses of a nucleic acid or a vector comprising the nucleic acid, and an amount of at least one bile acid or salt thereof at a concentration greater than the CMC of the bile acid or salt thereof, wherein the at least one bile acid or salt thereof is a compound selected from Table A.

In some embodiments, the composition comprises one or more doses of a nucleic acid or a vector comprising the nucleic acid, an amount of at least one bile acid or salt thereof, wherein the at least one bile acid or salt thereof is a compound selected from Table A, and an amount of one or more co-excipients described herein. In some embodiments, the composition comprises one or more doses of a nucleic acid or a vector comprising the nucleic acid, an amount of at least one bile acid or salt thereof at a concentration greater than the CMC of the bile acid or salt thereof, wherein the at least one bile acid or salt thereof is a compound selected from Table A, and an amount of one or more co-excipients described herein

In some embodiments, the composition comprises one or more doses of a therapeutic agent, wherein the therapeutic agent is a nucleic acid or a vector comprising the nucleic acid, and an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic. In some embodiments, the composition comprises one or more doses of a therapeutic agent, wherein the therapeutic agent is a nucleic acid or a vector comprising the nucleic acid, an amount of at least one bile acid or salt thereof at a concentration greater than the CMC of the bile acid or salt thereof, wherein the bile acid is selected from taurocholic acid, glycocholic acid, cholate, 3-[(3-Cholamidopropyl)dimethylammonio]-1-propanesulfonic acid, taurochenodeoxycholic acid, glycochenodeoxycholic acid, chenodeoxycholic, and an amount of one or more co-excipients described herein, wherein the one or more co-excipients are selected from a chelator, a polymer, a microparticle, a nanoparticle, a dendrimer, and a combination thereof.

In some embodiments, the nucleic acid or the vector is a class III compound according to the BCS. In some embodiments, the nucleic acid or the vector is a class IV compound according to the BCS. In some embodiments, the nucleic acid comprises RNA, DNA, or a combination thereof. In some embodiments, the vector is a viral vector comprising the nucleic acid (e.g., an adenoviral vector or retroviral vector comprising the nucleic acid). In some embodiments, the vector is a non-viral vector comprising the nucleic acid (e.g., a plasmid comprising the nucleic acid). In some embodiments, the nucleic acid is selected from an immunostimulatory oligonucleotide, an mRNA, a plasmid DNA, and an RNA interference (RNAi) oligonucleotide.

In some embodiments, the RNAi oligonucleotide is selected from an siRNA, a shRNA, a miRNA, and an antisense oligonucleotide (ASO). In some embodiments, the ASO is a gapmer.

In some embodiments, the nucleic acid comprises one or more chemical modifications. In some embodiments, the chemical modification is selected from a modified sugar moiety, a modified internucleoside linkage, a modified nucleobase, and a combination thereof.

In some embodiments, the compound of Formula (I) is a compound of Formula (I-1-a). In some embodiments, the compound of Formula (I) is a compound of Formula (I-1-b). In some embodiments, the compound of Formula (I) is a compound of Formula (I-1-c). In some embodiments, the compound of Formula (I) is a compound of Formula (I-1-d). In some embodiments, the compound of Formula (I) is a compound of Formula (I-1-e). In some embodiments, the compound of Formula (I) is a compound of Formula (I-1-f). In some embodiments, the compound of Formula (I) is a compound of Formula (I-2-a). In some embodiments, the compound of Formula (I) is a compound of Formula (I-2-b). In some embodiments, the compound of Formula (I) is a compound of Formula (I-2-c). In some embodiments, the compound of Formula (I) is a compound of Formula (I-2-d). In some embodiments, the compound of Formula (I) is a compound of Formula (I-2-e). In some embodiments, the compound of Formula (I) is a compound of Formula (I-2-f). In some embodiments, the compound of Formula (I) is a compound of Formula (I-1-a′). In some embodiments, the compound of Formula (I) is a compound of Formula (I-1-b′). In some embodiments, the compound of Formula (I) is a compound of Formula (I-1-c′). In some embodiments, the compound of Formula (I) is a compound of Formula (I-1-d′). In some embodiments, the compound of Formula (I) is a compound of Formula (I-1-e′). In some embodiments, the compound of Formula (I) is a compound of Formula (I-1-f). In some embodiments, the compound of Formula (I) is a compound of Formula (I-2-a′). In some embodiments, the compound of Formula (I) is a compound of Formula (I-2-b′). In some embodiments, the compound of Formula (I) is a compound of Formula (I-2-c′). In some embodiments, the compound of Formula (I) is a compound of Formula (I-2-d′). In some embodiments, the compound of Formula (I) is a compound of Formula (I-2-e′). In some embodiments, the compound of Formula (I) is a compound of Formula (I-2-f′).

In some embodiments, the one or more co-excipients comprises a chelator. In some embodiments, the chelator is selected from sodium ethyleneglycol-bis(Q-aminoethyl)-N,N,N′,N′-tetraacetate (EGTA), sodium ethylenedinitrilotetraacetate (EDTA), sodium glutamate (AAE), and a combination thereof.

In some embodiments, the one or more co-excipients comprises a polymer, e.g., a naturally occurring, a semi-synthetic, or a synthetic polymer. In some embodiments, the polymer comprises poly(methacrylate), poly(ethyl acrylate), poly(ethylene glycol), hyaluronic acid, polysaccharide, chitosan, arginine, poly(2-hydroxyethylmethacrylate), poly(N-isopropyl acrylamide), poly(ethylenimine), poly(N-(2-hydroxypropyl) methacrylamide), poly(glycolic acid), poly(lactic acid), poly(lactic-co-glycolic acid), poly(caprolactone), poly(orthoester), poly(anhydride), poly(amide), poly(ester amide), poly(phosphoester), poly(alky cyanoacrylate), or a combination thereof. In some embodiments, the polymer is selected from the polymer is selected from Eudragit E PO (EPO); Eudragit RL 100 (RL100); Poly(ethylene glycol) 1 kDa (PEG-1 kDa); Branched Poly(ethyleneimine) 800 Da (PEI:b0.8); Kollidon SR (KOLSR); Poly(methacrylic acid) 5 kDa (PMA:5); Poly(aspartic acid) 2 kDa (POLD:2); Poly(2-ethyloxazoline) 25 kDa (POXZ:25); Poly(glutamic acid) 50 kDa (POLE:50); Branched Poly(ethyleneimine)-co-Poly(ethylene glycol) 500 Da (bPEIPPEG:0.5); Vivacoat (VIVA).

In some embodiments, the one or more co-excipients comprises a dendrimer. In some embodiments, the dendrimer comprises a poly(amidoamine) (PAMAM), poly(propyleneimine), polyamide, polyether, polyester, or phosphorous-based architecture. In some embodiments, the dendrimer is G1, G2, G3, G4, G5, G6, G7, G8, G9, G10, or G11. In some embodiments, the dendrimer comprises a carboxylate terminus, an amino terminus, a hydroxy terminus, a succinamate terminus, a hydrophobe surface, or a combination thereof. In some embodiments, the dendrimer comprises a G1-G10 PAMAM architecture. In some embodiments, the dendrimer is selected from carboxylate terminal poly(amidoamine) dendrimer generation 3.5 (DENDAC3.5) and amine terminal poly(amidoamine) dendrimer generation 4 (DENDAM4).

In some embodiments, the one or more co-excipients comprises a microscopic particle (e.g., a microparticle or nanoparticle). In some embodiments, the particle is inorganic, carbon-based, ceramic, metal, polymeric, lipid-based, or a combination thereof. In some embodiments, the particle comprises a metal oxide, a metal salt, a mineral colloid, an inorganic salt, or a combination thereof. In some embodiments, the particle is a carbon nanoparticle. In some embodiments, particle is selected from calcium phosphate tribasic microparticles (CaPO₄); calcium phosphate dibasic microparticles (CaHPO₄); 5 μm spherical hydroxyapatite microparticles (HAP:5U); 2.5 μm spherical hydroxyapatite microparticles (HAP:2.5U); zinc oxide microparticles (ZNO:r10n); hydroxyapatite rod shaped nanoparticles 40 nm diameter (HAP:r40n); carbon black nanoparticles 13 nm diameter (CB:13n).

In some embodiments, the disclosure provides a dosage form of any one of the foregoing compositions, wherein the dosage form is formulated for enteral delivery (e.g., oral, duodenal, or rectal delivery). In some embodiments, the dosage form is a solid dosage form comprising the composition. In some embodiments, the solid dosage form is formulated for immediate release. In some embodiments, the solid dosage form is formulated for extended release. In some embodiments, the solid dosage form comprises an enteric coating. In some embodiments, the dosage form is a liquid dosage form comprising the composition.

In some embodiments, the dosage form comprises one or more doses of the therapeutic agent. In some embodiments, the dosage form comprises an effective amount of the therapeutic agent.

In some embodiments, the dosage form comprises an amount of the at least one bile acid or salt thereof sufficient to achieve an effective concentration at the site of absorption (e.g., the GI tract). In some embodiments, the effective concentration is greater than the CMC of the at least one bile acid or salt thereof. In some embodiments, the CMC is measured by a method selected from potentiometry, spectrophotometry, light scattering, and a combination thereof. In some embodiments, the effective concentration is at least about 2-fold, about 3-fold, about 4-fold, about 5-fold, about 6-fold, about 7-fold, about 8-fold, about 9-fold, or about 10-fold greater than the CMC. In some embodiments, the effective concentration is about 2-fold to about 200-fold, about 3-fold to about 200-fold, about 4-fold to about 200-fold, about 5-fold to about 200-fold, about 6-fold to about 200-fold, about 7-fold to about 200-fold, about 8-fold to about 200-fold, about 9-fold to about 200-fold, or about 10-fold to about 200-fold greater than the CMC. In some embodiments, the effective concentration is determined using a GI permeation assay described herein (e.g., a permeation assay using a GI-ORIS), wherein the effective concentration results in GI permeation (e.g., intestinal and/or stomach permeation) of the therapeutic agent that is increased compared to GI permeation of the therapeutic agent formulated as a control composition (e.g., a control composition lacking the at least one bile acid or salt thereof or the one or more co-excipients). In some embodiments, the effective concentration is determined using a GI permeation assay described herein (e.g., a permeation assay using a GI-ORIS), wherein the effective concentration results in intestinal permeation of the therapeutic agent that is increased compared to intestinal permeation of the therapeutic agent formulated as a control composition (e.g., a control composition lacking the at least one bile acid or salt thereof or the one or more co-excipients). In some embodiments, the effective concentration results in GI permeation (e.g., intestinal and/or stomach permeation) that is increased by about 1.1-fold, about 1.2-fold, about 1.3-fold, about 1.4-fold, about 1.5-fold, about 2-fold, about 3-fold, about 4-fold, about 5-fold, about 6-fold, about 7-fold, about 8-fold, about 9-fold, or about 10-fold compared to GI permeation of the therapeutic agent formulated as the control composition. In some embodiments, the effective concentration of the at least one bile acid or salt thereof is about 30 mg/mL to 200 mg/mL, about 40 mg/mL to 200 mg/mL, about 50 mg/mL to 200 mg/mL, about 60 mg/mL to 200 mg/mL, about 70 mg/mL to 200 mg/mL, about 80 mg/mL to 200 mg/mL, about 90 mg/mL to 200 mg/mL, or about 100 mg/mL to 200 mg/mL. In some embodiments, the amount of the at least one bile acid or salt thereof is substantially equivalent to the effective concentration (weight per volume) multiplied by a dilution factor described herein (e.g., about 2 mL to about 30 mL).

In some embodiments, the dosage form comprises the at least one bile acid or salt thereof in an amount of about 60 mg to about 2,000 mg. In some embodiments, the amount of the at least one bile acid or salt thereof is about 60 mg to about 1,000 mg. In some embodiments, the amount of the at least one bile acid or salt thereof is about 60 mg to about 600 mg. In some embodiments, the amount of the least one bile acid or salt thereof is about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the dosage form comprises an amount of the one or more co-excipients. In some embodiments, the amount is sufficient to achieve an effective concentration at the site of absorption (e.g., the GI tract). In some embodiments, the effective concentration is determined using a GI permeation assay described herein (e.g., a permeation assay using a G1-ORIS), wherein the effective concentration results in GI permeation of the therapeutic agent that is increased compared to GI permeation of the therapeutic agent formulated as a control composition (e.g., a control composition lacking the at least one bile acid or salt thereof or the one or more co-excipients). In some embodiments, the amount of the one or more co-excipients is substantially equivalent to the effective concentration (weight per volume) multiplied by a dilution factor described herein (e.g., about 2 mL to about 30 mL). In some embodiments, the dosage form comprises the one or more co-excipients in an amount of about 60 mg to about 2,000 mg. In some embodiments, the amount of the one or more co-excipients is about 60 mg to about 1,000 mg. In some embodiments, the amount of the one or more co-excipients is about 60 mg to about 600 mg. In some embodiments, the amount of one or more co-excipients is about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg.

In some embodiments, the dosage form comprises an amount of a chelator sufficient to achieve an effective concentration at the site of absorption of about 5 mg/mL to about 100 mg/mL. In some embodiments, the amount of the chelator is substantially equivalent to the effective concentration (weight per volume) multiplied by a dilution factor described herein (e.g., about 2 mL to about 30 mL). In some embodiments, the dosage form comprises the chelator in an amount of about 10 mg to about 1,500 mg. In some embodiments, the dosage form comprises the chelator in an amount of about 10 mg to about 1,000 mg. In some embodiments, the dosage form comprises the chelator in an amount of about 10 mg to about 500 mg. In some embodiments, the dosage form comprises the chelator in an amount of about 10 mg to about 250 mg. In some embodiments, the dosage form comprises the chelator in an amount of about 10 mg to about 100 mg.

In some embodiments, the dosage form comprises an amount of a polymer sufficient to achieve an effective concentration at the site of absorption of about 5 mg/mL to about 150 mg/mL. In some embodiments, the amount of the polymer is substantially equivalent to the effective concentration (weight per volume) multiplied by a dilution factor described herein (e.g., about 2 mL to about 30 mL). In some embodiments, the dosage form comprises the polymer in an amount of about 10 mg to about 1,500 mg. In some embodiments, the dosage form comprises the polymer in an amount of about 10 mg to about 1,000 mg. In some embodiments, the dosage form comprises the polymer in an amount of about 10 mg to about 500 mg. In some embodiments, the dosage form comprises the polymer in an amount of about 10 mg to about 250 mg. In some embodiments, the dosage form comprises the polymer in an amount of about 10 mg to about 100 mg.

In some embodiments, the dosage form comprises an amount of a microscopic particle (e.g., a microparticle or a nanoparticle) sufficient to achieve an effective concentration at the site of absorption of about 0.1 mg/mL to about 100 mg/mL. In some embodiments, the amount of the microscopic particle is substantially equivalent to the effective concentration (weight per volume) multiplied by a dilution factor described herein (e.g., about 2 mL to about 30 mL). In some embodiments, the dosage form comprises the microscopic particle in an amount of about 0.2 mg to about 1,500 mg. In some embodiments, the dosage form comprises the microscopic particle in an amount of about 0.2 mg to about 1,000 mg. In some embodiments, the dosage form comprises the microscopic particle in an amount of about 0.2 mg to about 500 mg. In some embodiments, the dosage form comprises the microscopic particle in an amount of about 0.2 mg to about 250 mg. In some embodiments, the dosage form comprises the microscopic particle in an amount of about 0.2 mg to about 100 mg. In some embodiments, the dosage form comprises the microscopic particle in an amount of about 0.2 mg to about 50 mg. In some embodiments, the dosage form comprises the microscopic particle in an amount of about 0.2 mg to about 20 mg. In some embodiments, the dosage form comprises the microscopic particle in an amount of about 0.2 mg to about 10 mg. In some embodiments, the dosage form comprises the microscopic particle in an amount of about 1 mg to about 10 mg.

### Methods of Enhanced Delivery to GI Tissue

The present disclosure provides methods of enhancing delivery of a therapeutic agent described herein to a subject, the method comprising administering to the subject a composition of the disclosure comprising the therapeutic agent, at least one bile acid or salt thereof described herein, and optionally one or more co-excipients described herein. In some embodiments, the composition has one or more properties for enhancing delivery to the subject compared to the therapeutic agent alone or a control composition comprising the therapeutic agent, but lacking the at least one bile acid or salt thereof or the optional one or more co-excipients. In some embodiments, the enhanced delivery results from improved GI permeation (e.g., intestinal and/or stomach) of the therapeutic agent compared to GI permeation of the therapeutic agent alone or a control composition comprising the therapeutic agent. In some embodiments, the enhanced delivery results from increased aqueous solubility of the therapeutic agent compared to the therapeutic agent alone or a control composition comprising the therapeutic agent.

Most orally administered compounds are primarily absorbed by the duodenum and jejunum in the upper parts of the GI tract. Absorption of compounds from the lumen of the GI tract requires passage through multiple layers including gastric juice, pericellular matrix, and mucous rich layer, to reach the epithelium, mucosa, and blood or lymph capillary walls. The epithelial cells lining the luminal side of the GI tract is a substantial barrier to drug delivery via oral administration. Epithelial cells are arranged in a single-column layer and intercalated with enterocytes. There are several routes of transport across the intestinal epithelium, including transcellular, paracellular, carrier-mediated and transcytotic transport pathways. Compounds delivered by the carrier mediated route rely on transport systems for nutrients located on the apical cell membrane. The transcytosis pathway enables active transport of compounds across cells. Passive diffusion through cells or between cells constitute the transcellular and paracellular pathways respectively. There are barriers to transport. For example, tight junctions restrict permeation between the cells by creating a seal between adjacent cells. Additional barriers to permeation of the intestinal epithelium include the presence of hydrolyzing enzymes at the apical surface of the epithelial membrane, the existence of an aqueous boundary layer on the surface of the epithelial membrane which may provide an additional diffusion barrier, the mucus layer associated with the aqueous boundary layer, and the acidic microenvironment that creates a proton gradient across the apical membrane. Absorption, and ultimately bioavailability, of a drug may also be reduced by other processes such as P-glycoprotein regulated transport of the drug back into die gut lumen and cytochrome P450 metabolism. The presence of food and/or beverages can also interfere with absorption and bioavailability. Without being bound by theory, compositions that increase passage of a therapeutic agent via one or more of these pathways result in enhanced delivery of the therapeutic agent from the GI tract into systemic circulation.

In some embodiments, the disclosure provides a method of enhancing delivery of a therapeutic agent to a subject, the method comprising formulating the therapeutic agent in a dosage form (e.g., a solid or liquid dosage form) comprising a composition of the disclosure and administering the dosage form to the GI tract of the subject (e.g., via enteral administration), wherein the composition comprises (i) one or more doses of the therapeutic agent, (ii) an amount of at least one bile acid or salt thereof described herein, wherein the amount is sufficient to achieve an effective concentration of the at least one bile acid or salt thereof at a site of absorption in the GI tract (e.g., wherein the effective concentration is determined using a GI permeation assay described herein, e.g., a permeation assay using a GI-ORIS); and optionally (iii) an amount of one or more co-excipients described herein, thereby enhancing delivery of the therapeutic agent to the subject.

In some embodiments, the GI permeation of the therapeutic agent is increased compared to GI permeation of the therapeutic agent alone or a control composition comprising the therapeutic agent (e.g., a composition lacking (ii) or (iii)). Methods for determining GI permeation of a therapeutic agent in a subject are known in the art and described herein (e.g., in vivo pharmacokinetic mass balance in a human, non-human primate, or other non-human animal; perfusion assay in a human, non-human primate, or other non-human animal; or in vitro using GI-ORIS, a transwell permeability assay, or a Ussing chamber assay).

In some embodiments, the GI permeation of the therapeutic agent is increased by at least about 1.1-fold, about 1.2-fold, about 1.3-fold, about 1.4-fold, about 1.5-fold, about 2-fold, about 3-fold, about 4-fold, about 5-fold, about 6-fold, about 7-fold, about 8-fold, about 9-fold, or about 10-fold compared to GI permeation of the therapeutic agent alone or a control composition comprising the therapeutic agent (e.g., a composition lacking (ii) or (iii)). In some embodiments, the GI permeation of the therapeutic agent is increased by about 1.1-fold to about 2-fold, about 1.1-fold to about 3-fold, about 1.5-fold to about 2-fold, about 1.5-fold to about 3-fold, about 1.5-fold to about 5-fold, about 2-fold to about 5-fold, about 2-fold to about 10-fold, about 5-fold to about 10-fold, about 5-fold to about 15-fold, about 5-fold to about 15-fold, about 5-fold to about 20-fold, about 5-fold to about 30-fold, about 5-fold to about 50-fold, about 10-fold to about 100-fold, about 10-fold to about 150-fold, or about 10-fold to about 200-fold compared to GI permeation of the therapeutic agent alone or a control composition comprising the therapeutic agent (e.g., a composition lacking (ii) or (iii)).

In some embodiments, the GI permeation of the compound is increased relative to GI permeation of a control composition, optionally wherein the control composition comprises the compound alone.

In some embodiments, the enteral administration is oral. In some embodiments, the enteral administration is via an enteral feeding tube. In some embodiments, the enteral administration is buccal. In some embodiments, the enteral administration is rectal.

### Methods of Enhanced Bioavailability

The present disclosure provides methods for increasing bioavailability of a therapeutic agent in a subject, the method comprising administering to the subject a composition of the disclosure comprising one or more doses of the therapeutic agent (e.g., an effective amount of the therapeutic agent), an amount of at least one bile acid or salt thereof described herein (e.g., an amount sufficient to achieve an effective concentration of the at least one bile acid or salt thereof at one or more sites of absorption in the GI tract), and optionally one or more co-excipients described herein. In some embodiments, the administering is enteral (e.g., oral, duodenal, or rectal). In some embodiments, the administering is dermal. In some embodiments, the administering is buccal or sublingual. In some embodiments, the administering is nasal (e.g., via inhalation). In some embodiments, the administering is ocular.

As used herein, the term “bioavailability” refers to the fraction of the one or more doses of the therapeutic agent administered to a subject that is available at the site of action following the administration. The term “site of action” refers to one or more bodily compartments and/or one or more tissue sites comprising the biological target(s) of the therapeutic agent, wherein modulation of the biological target(s) is effective for achieving a desired biological outcome (e.g., a desired therapeutic effect). In some embodiments, the amount of the therapeutic agent available at the site of action is considered to be equivalent to the amount in systemic circulation following the administration.

In some embodiments, the disclosure provides a method for increasing bioavailability of a therapeutic agent in a subject, the method comprising administering to the subject a composition of the disclosure comprising one or more doses of the therapeutic agent, an amount of at least one bile acid or salt thereof described herein, and optionally one or more co-excipients described herein. In some embodiments, the disclosure provides a method for increasing oral bioavailability of a therapeutic agent in a subject, the method comprising administering to the subject a composition of the disclosure comprising one or more doses of the therapeutic agent, an amount of at least one bile acid or salt thereof described herein, and optionally one or more co-excipients described herein. As used herein, the term “oral bioavailability” refers to the fraction of the one or more doses of the therapeutic agent administered to a subject orally that is available at the site of action following the administration. In some embodiments, oral bioavailability is the fraction of the one or more doses of the therapeutic agent administered to a subject orally that is in systemic circulation following the administration.

Methods to measure bioavailability (e.g., oral bioavailability) are known in the art (see, e.g., Olivares-Morales A, et al. Pharm Res. 2014 31:720-30; Currie G M. Pharmacology, Part 2: Introduction to Pharmacokinetics. J Nucl Med Technol. 2018 September; 46(3):221-230; Herkenne C, et al. Pharm Res. 2008 January; 25(1):87-103; Chow S C. Wiley Interdiscip Rev Comput Stat. 2014; 6(4):304-312). In some embodiments, bioavailability is determined by measuring the proportion of the one or more doses of the therapeutic agent present at the site of action following administration to a subject. In some embodiments, the measurement comprises obtaining a tissue sample from the subject and quantifying the amount of the therapeutic agent in the tissue sample. In some embodiments, the measurement comprises obtaining a fluid sample from the subject (e.g., a blood sample, a urine sample) and quantifying the amount of the therapeutic agent in the fluid sample. In some embodiments, bioavailability is calculated as the fraction of the dose amount of the therapeutic agent administered to the subject that reaches the systemic circulation in unchanged form. In some embodiments, this definition assumes that all or most of the dose amount administered to the subject that enters systemic circulation will reach the site of action.

In some embodiments, administration of a composition of the disclosure comprising one or more doses of a therapeutic agent, an amount of at least one bile acid or salt described herein, and optionally one or more co-excipients according to a route of administration described herein (e.g., enteral administration) results in a bioavailability of the therapeutic agent that is increased compared to bioavailability of a control composition comprising the one or more doses of the therapeutic agent, but lacking the at least one bile acid or salt or the optional one or more co-excipients. In some embodiments, the bioavailability (e.g., oral bioavailability) of the therapeutic agent is at least is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the bioavailability of the therapeutic agent when administered systemically, e.g., by intravenous injection or infusion. In some embodiments, the bioavailability (e.g., oral bioavailability) of the therapeutic agent is substantially equivalent to the bioavailability of the therapeutic agent when administered systemically, e.g., by intravenous injection or infusion.

In some embodiments, the amount of the therapeutic agent measured in systemic circulation following administration according to a route described herein (e.g., enteral administration) to a subject of a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the total amount of the therapeutic agent in the composition.

In some embodiments, the amount of the therapeutic agent measured in systemic circulation following oral administration (e.g., a single oral administration) to a subject of a composition of the disclosure is at least about 355%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the total amount of the therapeutic agent in the composition.

In some embodiments, the amount of the therapeutic agent measured in systemic circulation following administration according to a route described herein (e.g., enteral administration) to a subject of a dosage form (e.g., a solid or liquid dosage form) comprising a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the total amount of the therapeutic agent in the dosage form.

In some embodiments, the amount of the therapeutic agent measured in systemic circulation following oral administration (e.g., a single oral administration) to a subject of a dosage form (e.g., a solid or liquid dosage form) comprising a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the total amount of the therapeutic agent in the dosage form.

In some embodiments, the bioavailability of the therapeutic agent following administration according to a route described herein (e.g., enteral administration) of a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99%.

In some embodiments, the bioavailability of the therapeutic agent following oral administration (e.g., a single oral administration) of a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99%.

In some embodiments, the bioavailability of the therapeutic agent following administration according to a route described herein (e.g., enteral administration) of a dosage form (e.g., solid or liquid dosage form) comprising a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99%.

In some embodiments, the bioavailability of the therapeutic agent following oral administration (e.g., a single oral administration) of a dosage form (e.g., solid or liquid dosage form) comprising a composition of the disclosure is at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99%.

In some embodiments, administration of a composition of the disclosure comprising one or more doses of a therapeutic agent, an amount of at least one bile acid or salt described herein, and optionally one or more co-excipients according to a route of administration described herein (e.g., enteral administration) results in an increased Cmax of the therapeutic agent as compared to administration of a control composition comprising the one or more doses of the therapeutic agent, but lacking the at least one bile acid or salt thereof and the optional one or more co-excipients. As used herein, the term “Cmax” refers to the maximum observed plasma concentration of the therapeutic agent following the administration. In some embodiments, administration of a dosage form (e.g., solid or liquid dosage form) comprising the composition according to a route of administration described herein (e.g., enteral administration) results in an increased Cmax of the therapeutic agent as compared to a dosage form comprising the control composition. In some embodiments, the Cmax of the therapeutic agent is increased by at least about 1.5-fold, about 2-fold, about 3-fold, about 4-fold, about 5-fold, about 10-fold, about 15-fold, or about 20-fold compared to administration of the control composition or a dosage form comprising the control composition.

In some embodiments, administration of a composition of the disclosure comprising one or more doses of a therapeutic agent, an amount of at least one bile acid or salt described herein, and optionally one or more co-excipients according to a route of administration described herein (e.g., enteral administration) results in an increased Tmax of the therapeutic agent as compared to administration of a control composition comprising the one or more doses of the therapeutic agent, but lacking the at least one bile acid or salt thereof and the optional one or more co-excipients. As used herein, the term “Tmax” refers to the time point of maximum observed plasma concentration following administration. In some embodiments, administration of a dosage form (e.g., solid or liquid dosage form) comprising the composition according to a route of administration described herein (e.g., enteral administration) results in an increased Tmax of the therapeutic agent as compared to a dosage form comprising the control composition. In some embodiments, the Tmax of the therapeutic agent is increased by at least about 1.5-fold, about 2-fold, about 3-fold, about 4-fold, about 5-fold, about 10-fold, about 15-fold, or about 20-fold compared to administration of the control composition or a dosage form comprising the control composition.

In some embodiments, administration of a composition of the disclosure comprising one or more doses of a therapeutic agent, an amount of at least one bile acid or salt described herein, and optionally one or more co-excipients according to a route of administration described herein (e.g., enteral administration) results in an average AUC of the therapeutic agent that is increased compared to the average AUC following systemic administration (e.g., via intravenous injection) of the composition or administration via the same route of a control composition comprising the one or more doses of the therapeutic agent, but lacking the at least one bile acid or salt thereof and the optional one or more co-excipients. As used herein, the term “AUC” refers to the “area under the curve” of a plot of concentration of a therapeutic agent present in a tissue sample (e.g., a blood sample) obtained from subject following administration of the therapeutic agent versus time. As is understood by the skilled artisan, AUC is determined using mathematical approaches that calculate the concentration from time interval zero to infinity (“AUC total”) or a predetermined time point (e.g., calculation of concentration over a time interval of zero to 7 days provides “AUC0-7”). In some embodiments, administration of a dosage form (e.g., solid or liquid dosage form) comprising a composition of the disclosure according to a route of administration described herein (e.g., enteral administration) results in an average AUC of the therapeutic agent that is increased compared to the average AUC following systemic administration (e.g., via intravenous injection) of the composition or administration via the same route of a dosage form comprising a control composition.

In some embodiments, the AUC of a composition of a disclosure following administration according to a route of administration described herein (e.g., enteral administration) is at least about 1.5-fold, about 2-fold, about 3-fold, about 4-fold, about 5-fold, about 10-fold, about 15-fold, or about 20-fold of the AUC of the composition following systemic administration (e.g., via intravenous injection) of the composition. In some embodiments, the AUC of a composition of a disclosure following administration according to a route of administration described herein (e.g., enteral administration) is at least about 1.5-fold, about 2-fold, about 3-fold, about 4-fold, about 5-fold, about 10-fold, about 15-fold, or about 20-fold of the AUC of the composition following administration via the same route of a control composition. In some embodiments, the AUC of a dosage form (e.g., solid, semi-solid, or liquid dosage form) comprising the composition of the disclosure following administration according to a route of administration described herein (e.g., enteral administration) is at least about 1.5-fold, about 2-fold, about 3-fold, about 4-fold, about 5-fold, about 10-fold, about 15-fold, or about 20-fold of the AUC of the composition following systemic administration (e.g., via intravenous injection) of the composition. In some embodiments, the AUC of a dosage form (e.g., solid, semi-solid, or liquid dosage form) comprising the composition of the disclosure following administration according to a route of administration described herein (e.g., enteral administration) is at least about 1.5-fold, about 2-fold, about 3-fold, about 4-fold, about 5-fold, about 10-fold, about 15-fold, or about 20-fold of the AUC following administration via the same route of a dosage from comprising a control composition.

In some embodiments, the percent bioavailability of a therapeutic agent is increased when administered as a composition of the disclosure as compared to a control composition lacking the at least one bile acid or salt thereof and the optional one or more co-excipients. As used herein, the term “percent bioavailability” (also referred to as “F”) represents the fraction of the amount of the therapeutic agent measured in circulation when enterically administered (e.g., as an oral dose) as compared to the amount of the therapeutic agent measured in circulation when administered systemically (e.g., by intravenous injection). The calculation of F is determined by measuring the AUC (e.g., total AUC) for the therapeutic agent when enterically administered (the “test route”) compared to the AUC when administered systemically (“systemic route”). For example, in some embodiments, the following equation is used to calculate percent bioavailability: F (%)=AUCₜₒₜₐₗ (test route)/AUCₜₒₜₐₗ (systemic route). This is an important term that establishes the relative fraction of the therapeutic agent entering circulation via the test route compared to the maximum possible amount entering circulation via the systemic route. In some embodiments, the percent bioavailability of a therapeutic agent administered as a composition of the disclosure is increased by at least about 1.5-fold, about 2-fold, about 3-fold, about 4-fold, about 5-fold, about 10-fold, about 15-fold, or about 20-fold compared to percent bioavailability of the therapeutic agent administered as a control composition (e.g., a composition lacking the at least one bile acid or salt thereof and the optional one or more co-excipients). In some embodiments, the percent bioavailability of a therapeutic agent administered as a dosage form (e.g., a solid, semi-solid, or liquid dosage form) comprising a composition of the disclosure is increased by at least about 1.5-fold, about 2-fold, about 3-fold, about 4-fold, about 5-fold, about 10-fold, about 15-fold, or about 20-fold compared to percent bioavailability of the therapeutic agent administered as dosage form comprising a control composition (e.g., a composition lacking the at least one bile acid or salt thereof and the optional one or more co-excipients).

### Pharmaceutical Compositions

The present disclosure provides pharmaceutical compositions comprising a composition described herein, and a pharmaceutically acceptable carrier.

In some embodiments, the pharmaceutical composition is formulated as a dosage form suitable for administration according to a route of administration described herein. In some embodiments, the route of administration comprises enteral administration (e.g., oral, duodenal, or rectal administration). In some embodiments, the pharmaceutical composition is formulated as a dosage form suitable for administration across a skin or mucosal barrier (e.g., nasal, ocular, or dermal administration) of the subject. In some embodiments, the pharmaceutical composition is formulated as a solid dosage form for enteral administration (e.g., oral, duodenal, or rectal administration) to the subject. In some embodiments, the pharmaceutical composition is formulated as a solid dosage form for administration across a skin or mucosal barrier (e.g., nasal, ocular, or dermal administration) of the subject. In some embodiments, the pharmaceutical composition is formulated as a suspension for enteral administration (e.g., oral, duodenal, or rectal administration) to the subject. In some embodiments, the pharmaceutical composition is formulated as a suspension for administration across a skin or mucosal barrier (e.g., nasal, ocular, or dermal administration) of the subject. In some embodiments, the pharmaceutical composition is formulated as a liquid dosage form for enteral administration (e.g., oral, duodenal, or rectal administration) to the subject. In some embodiments, the pharmaceutical composition is formulated in a dosage form convenient for storage, wherein when the composition is reconstituted in an aqueous solution, the dosage form dissolves and yields a suspension suitable for enteral administration (e.g., oral, duodenal, or rectal administration) to a subject. For example, in some embodiments, the pharmaceutical composition is manufactured in a concentration form (e.g., as a tablet, suspension tablet, or effervescent tablet or powder) and reconstituted with water or other diluent to generate an aqueous form for enteral administration (e.g., oral, duodenal, or rectal administration) to a subject. In some embodiments, the concentrated form is suitable for enteral administration (e.g., oral, duodenal, or rectal administration) to a subject. In some embodiments, the pharmaceutical composition is formulated in a dosage form for inhalation.

Pharmaceutically acceptable carriers for use in the pharmaceutical compositions described herein are known in the art, see e.g., Remington's Pharmaceutical Sciences, 17th Edition, 1985. In some embodiments, the pharmaceutically acceptable carrier is a liquid for dissolving or dispersing a composition of the disclosure. In some embodiments, the liquid is aqueous, non-aqueous, or a combination thereof. Non-aqueous solvents/cosolvents may be added to increase the solubility of poorly soluble substances and enhance the chemical stability of a therapeutic agent. Suitable solvents/co-solvents, solubilizers or vehicles, include, but are not limited to, dichloromethane, acetonitrile, ethyl acetate, acetone, propylene carbonate, water, glycerin, coconut fatty acid diethanolamide, medium and/or long chain fatty acids or glycerides, monoglycerides, diglycerides, triglycerides, structured triglycerides, soyabean oil, peanut oil, corn oil, corn oil monoglycerides, corn oil diglycerides, corn oil triglycerides, polyethylene glycol, caprylocaproylmacroglycerides, caproyl 90, propylene glycol, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, acetone, methyl isobutyl ketone, and methyl ethyl ketone.

In some embodiments, the pharmaceutical composition comprises a buffering agent. As used herein the term “buffering agent” refers to a pharmaceutically acceptable weak base or strong base (and mixtures thereof) that, when formulated or delivered with (e.g., before, during and/or after) the composition, functions to substantially prevent or inhibit the acid-mediated degradation of the therapeutic agent by gastric acid, thereby preserving the bioavailability of the therapeutic agent following oral administration. In some embodiments, the buffering agent is administered in an amount sufficient to render a pH in the stomach that improves the bioavailability of the therapeutic agent following oral administration. In some embodiments, the buffering agent comprises any one or any combination selected from sodium bicarbonate, potassium bicarbonate, magnesium hydroxide, magnesium lactate, magnesium glucomate, aluminum hydroxide, aluminum hydroxide/sodium bicarbonate coprecipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium cholride, calcium hydroxide, calcium lactate, calcium carbonate, calcium bicarbonate, and other calcium salts.

In some embodiments, the pharmaceutical composition comprises an additive to enhance stability, sterility, and/or isotonicity. For example, in some embodiments, the pharmaceutical composition comprises an antimicrobial preservative and/or an antioxidant. Antibacterial and antifungal agents are known in the art. Non-limiting examples include parabens, chlorobutanol, phenol, and sorbic acid. In some embodiments, the pharmaceutical composition comprises an isotonic agent (e.g., a sugar or sodium chloride). In some embodiments, the pharmaceutical composition comprises a thickening agent (e.g., methylcellulose) to promote stability of the therapeutic agent in a solution. In some embodiments, the pharmaceutical composition comprises a flavoring agent. In some embodiments, the pharmaceutical composition comprises an anti-foaming agent (e.g., simethicone 80 mg, Mylicon®).

### Solid Dosage Form

In some embodiments, a pharmaceutical composition described herein is formulated as a solid dosage form (e.g., a capsule or a tablet) for administration according to a route of administration described herein. In some embodiments, the route of administration comprises enteral administration (e.g., oral, duodenal, or rectal administration). In some embodiments, a pharmaceutical composition described herein is formulated as a solid dosage form (e.g., a capsule or a tablet) for administration across a skin or mucosal barrier (e.g., nasal, ocular, or dermal administration).

As used herein, the term “solid dosage form” refers to a dosage form that is a solid at ambient conditions. In some embodiments, the solid dosage form comprises a therapeutic agent in a substantially amorphous form. In some embodiments, solid dosage form comprises the therapeutic agent as a substantially crystalline form. In some embodiments, solid dosage form comprises the therapeutic agent as mixture of an amorphous and crystalline form.

In some embodiments, the pharmaceutical composition is formulated as an enteric-coated solid dosage form (as either a delayed-release capsule or tablet). In some embodiments, the pharmaceutical composition is formulated as a semi-solid dosage form for enteral administration (e.g., oral, duodenal, or rectal administration).

In some embodiments, the pharmaceutical composition is formulated as a solid dosage from for controlled drug delivery. As used herein, “controlled drug delivery” refers to release or administration of a therapeutic agent from a solid dosage form in a controlled fashion in order to achieve the desired pharmacokinetic profile in vivo. An aspect of controlled drug delivery is the ability to manipulate the dosage form in order to establish the desired kinetics of drug release. In some embodiments, the pharmaceutical composition is formulated as a solid dosage form for sustained drug delivery, wherein the release of the therapeutic agent from the formulation is protracted over a period of time, e.g., minutes, hours, days, weeks, or months. In some embodiments, the release of the therapeutic agent provides consistent levels of the therapeutic agent over a time period ranging from a few minutes to a day. In some embodiments, the release profile is characterized by the absence of an immediate release phase, such as one obtained from intravenous administration.

In some embodiments, the solid dosage form is formulated for immediate release. As used herein, the term “immediate release” in reference to a solid dosage form of the disclosure means that the solid dosage form dissolves without substantially prolonging release of a therapeutic agent contained therein (e.g., upon exposure to gastrointestinal dissolution conditions or following oral administration). As used herein, the term “gastrointestinal dissolution conditions” refers to a dissolution test as defined by the USP (see, e.g., The United States Pharmocopeial Convention, 2011, world wide web: ftp.uspbpep.com/v29240/usp29nf24s0_c711 html). In some embodiments, the dissolution test comprises exposing the solid dosage form to release medium (e.g., 900 mL 0.1 M HCl in a USP Apparatus 1 (basket) or Apparatus 2 (paddle) at about 37° C. and about 100 rpm). In some embodiments, the solid dosage form formulated for immediate release is characterized by release of at least about 85% of a therapeutic agent contained therein in less than about 60 minutes under gastrointestinal dissolution conditions. In some embodiments, the solid dosage form formulated for immediate release undergoes rapid dissolution following oral administration to release the therapeutic agent contained therein for gastrointestinal absorption. In some embodiments, the solid dosage form formulated for immediate release is characterized by at least about 70%, about 75%, about 80%, or higher release of an active ingredient contained therein within about 30 minutes, about 45 minutes, about 60 minutes, or about 90 minutes following oral administration.

In some embodiments, the solid dosage form is formulated for extended or delayed release. As used herein, the term “extended release” in reference to a solid dosage form of the disclosure means that the solid dosage form releases a therapeutic agent contained therein over an extended period of time (e.g., upon exposure to gastrointestinal dissolution conditions or following oral administration). In some embodiments, the solid dosage form formulated for extended release is characterized by release of a therapeutic agent contained therein at a predetermined rate following oral administration. In some embodiments, the solid dosage form formulated for extended release is characterized by release of a therapeutic agent contained therein at a predetermined location in the gastrointestinal tract following oral administration (e.g., in the stomach or small intestine). In some embodiments, the solid dosage form formulated for extended release is characterized by release of at least about 85% of an active ingredient contained therein in greater than about 60 minutes, 90 minutes, 120 minutes, or 180 minutes under gastrointestinal dissolution conditions. In some embodiments, the extended release is pulsatile. In some embodiments, the solid dosage form comprises an enteric coating that provides for extended release.

In some embodiments, a solid oral dosage form is a tablet, a multiparticulate, or a capsule. In some embodiments, the solid oral dosage form is a delayed release dosage form, wherein the release of the therapeutic agent, the one or more bile acids or salt thereof, and the optional one or more co-excipients drug in the GI tract is protracted over a period of time. In some embodiments, the solid oral dosage form is selected to provide a release profile that minimizes the dilution of the one or more bile acids or salts thereof in the GI tract. In some embodiments, the solid oral dosage form is a delayed release rapid onset dosage form. Such a dosage form minimizes the release of the composition components (i.e., the therapeutic agent, one or more bile acids or salts thereof, and optional one or more co-excipients) in the stomach, but releases the components rapidly in the intestine, thereby maximizing the concentration of the components at the site of absorption.

In some embodiments, the solid dosage form comprises a disintegrant to increase solubility following reconstitution. In some embodiments, the solid dosage form comprises one or more additives to increase compressibility (e.g., microcrystalline cellulose). In some embodiments, the solid dosage form comprises any one or any combination selected from a flavoring agent, a sweetening agent, a flow aid, a lubricant, a colorant, a diluent, a buffering agent, a moistening agent, a preservative, a pharmaceutically acceptable carrier, and a disintegrants. In some embodiments, the solid dosage form is a powder, a tablet, a capsule, pelleted form, a lozenge, a troche, or an effervescing tablet.

As used herein, the term “tablet” includes, but is not limited to, immediate release (IR) tablets, sustained release (SR) tablets, matrix tablets, multilayer tablets, multilayer matrix tablets, extended release tablets, delayed release tablets and pulsed release tablets. In some embodiments, the tablet comprises one or more coating materials, e.g., polymer coating materials, such as enteric coatings, rate-controlling coatings, and semi-permeable coatings.

As used herein, the term “capsule” includes instant release capsules, sustained release capsules, coated instant release capsules, coated sustained release capsules, delayed release capsules and coated delayed release capsules. In some embodiments, the solid dosage form is an enteric coated capsule dosage form. In some embodiments, the solid dosage form is an enteric coated rapid onset capsule dosage form.

In some embodiments, the solid dosage form is multiparticulate. As used herein, the term “multiparticulate” as used herein means a plurality of discrete particles, pellets, mini-tablets and mixtures or combinations thereof. In some embodiments, the solid dosage from comprises a hard or soft gelatin capsule encapsulating the multiparticulate. In some embodiments, the solid dosage from comprises a sachet comprising the multiparticulate. In some embodiments, the solid dosage form comprises a multiparticulate coated with a layer comprising a rate controlling polymer material. In some embodiments, the solid dosage form comprises a multiparticulate comprising at least two types of particles with different release properties. For example, in some embodiments, the multiparticulate dosage form comprises a blend of an instant release component and a delayed release component contained in a capsule. In some embodiments, the multiparticulate dosage form comprises a capsule containing delayed release rapid onset minitablets. In some embodiments, the multiparticulate dosage form comprises a delayed release capsule comprising instant release minitablets. In some embodiments, the multiparticulate dosage form comprises a capsule comprising delayed release granules. In some embodiments, the multiparticulate dosage form comprises a delayed release capsule comprising instant release granules.

In some embodiments, the solid dosage form is compressed or comprises a compressed tablet. The term “compressed tablet” generally refers to a plain, uncoated tablet for oral ingestion, prepared by a single compression or by pre-compaction tapping followed by a final compression. In some embodiments, the solid dosage form comprises a coating. For example, in some embodiments, the solid dosage form comprises a coating that dissolves upon ingestion or upon contact with diluent.

Methods for manufacturing solid dosage forms are known in the art. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986).

In some embodiments, the solid dosage form comprises a controlled release modifier. A “controlled release modifier” refers to a substance that upon hydration of the dosage form will preferentially interact with the therapeutic agent to reduce its rate of diffusion from the dosage form. Such excipients may also reduce the rate of water uptake by the formulation and thus enable a more prolonged dissolution and release of the therapeutic agent. In some embodiments, the controlled release modifiers are capable of binding molecularly to the therapeutic agent via physical (and therefore reversible) interactions, thus increasing the effective molecular weight of the therapeutic agent and thus further modifying its permeation (diffusion) characteristics through the epithelial and basal membranes of the sublingual mucosa. Such binding is reversible in nature and does not involve any chemical modifications of the therapeutic agent. In some embodiments, the controlled release modifier upon hydration forms a three-dimensional structure that entraps the therapeutic agent and prolongs its release from the dosage form. Exemplary controlled release modifiers are selected from lipids, phospholipids, sterols, surfactants, polymers and salts.

In some embodiments, the solid dosage form comprises a filler or bulking agent. Exemplary bulking agents include, but are not limited to, lactose USP, Starch 1500, mannitol, sorbitol, malitol or other non-reducing sugars; microcrystalline cellulose (e.g., Avicel), dibasic calcium phosphate dehydrate, sucrose, and mixtures thereof.

In some embodiments, the solid dosage form comprises a solubilizing agent. In some embodiments, the solubilizing agent improves the solubility of the therapeutic agent, while facilitating ease of handling and manufacturing. Exemplary solubilizers include, but are not limited to, cyclodextrins, pH adjusters, salts and buffers, surfactants, fatty acids, phospholipids, and metals of fatty acids. Exemplary surfactants include, but are not limited to, ionic (sodium lauryl sulfate, etc), non-ionic such as polysorbates (Tween and Span surfactant series, Poloxamers, etc.), bile salts (such as sodium taurocholate, sodium taurodeoxycholate, sodium glycodeoxycholate, sodium glycocholate, etc), various alkyl glycosides, fatty acids, phosphatidylcholines, triglycerides, sphingolipids, glycosylated lipids, PEGylated lipids and mixtures thereof. Exemplary metal salts and buffers include, but are not limited to, organic (e.g., acetate, citrate, tartrate) or inorganic (e.g., phosphate, carbonate, bicarbonate, borate, sulfate, sulfite, bisulfite, metabisulfite, chloride) salts of metals (e.g., sodium, potassium, calcium, magnesium).

In some embodiments, the solid dosage form comprises an additive to stabilize the therapeutic agent from chemical of physical degradation. Such degradation reactions may include oxidation, hydrolysis, aggregation, deamidation, etc. Appropriate excipients that can stabilize the drug substance may include anti-oxidants (e.g., BHT, BHA, vitamins, citric acid, EDTA, sodium bisulfate, sodium metabisulfate, thiourea, methionine), anti-hydrolytic agents, and aggregation-blockers (e.g., surfactants, amino-acids, such as arginine, glycine, histidine, methionine).

In some embodiments, the solid dosage form comprises an enteric coating, such as any enteric coating known in the art. In some embodiments, the enteric coating comprises a polymer that is preferentially soluble in the less acidic environment of the intestine relative to the more acidic environment of the stomach (e.g., a polymer more soluble at pH greater than about 5 or 6 than at a pH of less than about 5). In some embodiments, the polymer is selected from a cellulose derivative (e.g., hydroxypropylmethylcellulose (HPMC), hydroxypropylcellulose (HPC)) or ethylcellulose); polyvinylpyrrolidones (PVP); polyethylene glycols (PEG); polyvinyl alcohols (PVA); acrylates (e.g., polymethacrylate (e.g., Eudragit® E)); cyclodextrins (e.g., β-cyclodextin) and copolymers and derivatives thereof.

### Liquid Oral Formulations

In some embodiments, the pharmaceutical composition is formulated as a liquid dosage form for administration according to a route of administration described herein. In some embodiments, the route of administration comprises enteral administration (e.g., oral, duodenal, or rectal administration). In some embodiments, the pharmaceutical composition is formulated as a liquid dosage form for administration across a skin or mucosal barrier (e.g., nasal, ocular, or dermal administration). As used herein, the term “liquid dosage form” refers to a dosage form able to flow at ambient temperature and includes solutions, suspensions and emulsions.

In some embodiments, the liquid dosage form comprises a wetting agent. In some embodiments, the liquid dosage form comprises a buffering agent. In some embodiments, the liquid dosage form comprises a viscosity modifying agent. In some embodiments, the liquid dosage form comprises an anti-foaming agent. In some embodiments, the liquid dosage form comprises a preservative. In some embodiments, the liquid dosage form comprises a sweetener. In some embodiments, the liquid dosage form comprises a sweetening agent and/or flavoring agent.

In some embodiments, the liquid dosage form comprises a liquid dispersion, a suspension, a solution, an emulsion, a spray, a syrup, an elixir, a drop, a concentrate, or a combination thereof. In some embodiments, the liquid dosage form comprises a dry powder to be prepared as a liquid prior to administration to a patient. In some embodiments, the liquid dosage form comprises one or more pharmaceutically acceptable excipients or additives selected from the group comprising of vehicles, solvents/co-solvents and/or solubilizers, pH adjusting agents and/or pH modifying agents and/or buffering agents or any combination thereof. In some embodiments, the liquid dosage form comprises an anti-microbial agents or preserving agents or preservatives.

In some embodiments, the liquid dosage form is an immediate release dosage forms or modified release dosage forms, such as extended release, controlled release, sustained release, prolonged release and delayed release. In some embodiments, the liquid dosage form comprises one or more suitable excipients or additives for the preparation of modified release dosage forms such as rate controlling polymers.

In some embodiments, the liquid dosage form comprises a wetting agents, e.g., to create a homogeneous dispersion of solid particles in a liquid vehicle. For an aqueous vehicle, alcohol, glycerin, and PG are frequently used to facilitate the removal of adsorbed air from the surface of particles. For a non-aqueous liquid vehicle, mineral oil is commonly used as a wetting agent. Non-limiting examples of wetting agents are Benzalkonium chloride, Benzethonium chloride, Cetylpyridinium chloride, Docusate sodium, Nonoxynol 9, Octoxynol, Poloxamer, Poloxamer 124, Poloxamer 188, 237, 338, 407, Polyoxyl 35 castor oil, Polyoxyl 40 hydrogenated castor oil, Polyoxyl 10 oleyl ether, Polyoxyl 20 cetylstearyl ether, Polyoxyl 40 stearate, Polysorbate 20, Polysorbate 40, Polysorbate 60, Polysorbate 80, Sodium lauryl sulfate, Sorbitan monolaurate, Sorbitan monooleate, Sorbitan monopalmitate, Sorbitan monostearate, Tyloxapol, and combinations thereof. In some of embodiments, the liquid dosage form comprises a solubility enhancing agents e.g., DL-methionine, caffeine, nicotinamide, vanillin, benzyl alcohol, ethanol and diethylene glycol monoethyl ether and the like or combinations thereof. In some of embodiments, the liquid dosage form comprises a stabilizing agent, e.g., sodium metabisulphite, sodium bisulphite, ethylene diamine tetraacetic acid (EDTA) or salts thereof, ascorbic acid and the like or combinations thereof. In some of embodiments, the liquid dosage form comprises a mucoadhesive, e.g., hydroxypropyl cellulose, gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, xanthan gum, alginate, copolymers of dextran, polyacrylamide, acacia, copolymer of caprolactone and ethylene oxide, carbopol 934, tragacanth, eudragit and combinations thereof. In some of embodiments, the liquid dosage form comprises a viscosity modifying agent, e.g., to improve suspension, impart viscosity, and/or to reduce particle sedimentation. Viscosity modifying agents can be classified into cellulose derivatives, clays, natural gums, and synthetic gums. Non-limiting examples of viscosity modifying agents are Acacia, Agar, Alginic acid, Carbomer, Carmellose sodium, Dextrin, Gelatin, Veegum or Gel white, Gellan gum, Sodium alginate, Methylcellulose, Hydroxyethyl cellulose, Hydroxypropyl cellulose, Hydroxypropylmethyl cellulose, Hydroxypropyl starch, Hypromellose, Maltodextrin, Methylcellulose, Modified starch, Pectin, Poloxamer, Polycarbophil, Polyethylene glycol, Polyvinyl acetate, Poly (vinyl alcohol), Potassium alginate, Polyvinyl pyrrolidone, Pregelatinized starch, Propylene glycol alginate, Sodium alginate, Carboxymethyl cellulose or an alkali metal salt thereof, Microcrystalline cellulose, gum Arabic, Karaya gum, Sterculia gum, Tragacanth, Xanthan gum, Bentonite, Carageenan, Guar gum, Colloidal silicon dioxide and the like or any combinations thereof.

In some embodiments, the liquid dosage form comprises a pH adjusting agent, e.g., Acetic acid, Adipic acid, Ammonium carbonate, Ammonium hydroxide, Ammonium phosphate, Boric acid, Citric acid, Diethanolamine, Fumaric acid, Hydrochloric acid, Malic acid, Nitric acid, Propionic acid, Potassium acetate, Potassium bicarbonate, Potassium chloride, Potassium citrate, Potassium metaphosphate, Potassium phosphate, Sodium acetate, Sodium bicarbonate, Sodium borate, Sodium carbonate, Sodium chloride, Sodium citrate, Sodium glycolate, Sodium hydroxide, Sodium lactate, Sodium phosphate, Sodium proprionate, Succinic acid, Sulfuric acid, Tartaric acid, Triethylamine, Triethanolamine, Tromethamine, Trolamine and combinations thereof.

### Kits

The present disclosure provides kits for performing the methods described herein. In some embodiments, the kit comprises a composition of the disclosure (e.g., a composition comprising one or more doses of a therapeutic agent, an amount of at least one bile acid or salt thereof, and optionally one or more co-excipients) or a pharmaceutical composition thereof (e.g., formulated as a solid or liquid dosage form) and a package insert comprising instructions for administration of the composition or pharmaceutical composition to a subject according to a route of administration described herein for preventing, ameliorating, or treating a disease or disorder in the subject. In some embodiments, the route of administration comprises enteral administration (e.g., oral administration).

In some embodiments, the kit comprises a composition of the disclosure (e.g., a composition comprising one or more doses of a therapeutic agent, an amount of at least one bile acid or salt thereof, and optionally one or more co-excipients) or a pharmaceutical composition thereof (e.g., formulated as a solid or liquid dosage form) and a package insert comprising instructions for administration (e.g., nasal, ocular, or dermal administration) of the composition or pharmaceutical composition across a skin or mucosal barrier of a subject for preventing, ameliorating, or treating a disease or disorder in the subject.

In some embodiments, the kit comprises a container comprising the composition or the pharmaceutical composition. In some embodiments, the composition or the pharmaceutical composition is contained in a pharmaceutically-acceptable package, container, pump, bottle with spray pump, bottle with dropper assembly, bottle, collapsible tube, glass ampoule, stoppered vial, or pre-filled syringe.

In some embodiments, the kit comprises a container comprising the composition or the pharmaceutical composition, a means for administration of the composition or pharmaceutical composition to a subject (e.g., a syringe), and a package insert comprising instructions for administration of the composition or the pharmaceutical composition to the subject according to a route of administration described herein to prevent, ameliorate, or treat a disease or disorder. In some embodiments, the kit comprises a medicament comprising the composition or the pharmaceutical composition, a means for administration of the medicament to a subject (e.g., a syringe), and a package insert comprising instructions for administration of the medicament to the subject according to a route of administration described herein to prevent, ameliorate, or treat a disease or disorder.

In some embodiments, the kit comprises a container comprising the composition or the pharmaceutical composition, a means for enteral administration of the composition or pharmaceutical composition to a subject (e.g., a syringe), and a package insert comprising instructions for enteral administration of the composition or the pharmaceutical composition to the subject to prevent, ameliorate, or treat a disease or disorder. In some embodiments, the kit comprises a medicament comprising the composition or the pharmaceutical composition, a means for enteral administration of the medicament to a subject (e.g., a syringe), and a package insert comprising instructions for administration of the medicament to the subject to prevent, ameliorate, or treat a disease or disorder.

In some embodiments, the kit comprises a container comprising the composition or the pharmaceutical composition, a means for administration (e.g., nasal, ocular, or dermal administration) of the composition or pharmaceutical composition, and a package insert comprising instructions for administration (e.g., nasal, ocular, or dermal administration) of the composition or the pharmaceutical composition across a mucosal or skin barrier of a subject to prevent, ameliorate, or treat a disease or disorder. In some embodiments, the kit comprises a medicament comprising the composition or the pharmaceutical composition, a means for administration of the medicament, and a package insert comprising instructions for administration (e.g., nasal, ocular, or dermal administration) of the medicament across a mucosal or skin barrier of a subject to prevent, ameliorate, or treat a disease or disorder.

In some embodiments, the kit comprises a container comprising the composition or the pharmaceutical composition, and a package insert comprising instructions for enteral administration (e.g., oral, duodenal, or rectal administration) of the composition or the pharmaceutical composition to a subject to prevent, ameliorate, or treat a disease or disorder. In some embodiments, the kit comprises a medicament comprising the composition or the pharmaceutical composition, and a package insert comprising instructions for enteral administration (e.g., oral, duodenal, or rectal administration) of the medicament to a subject to prevent, ameliorate, or treat a disease or disorder.

In some embodiments, the kit comprises a container comprising the composition or the pharmaceutical composition, and a package insert comprising instructions for administration (e.g., nasal, ocular, or dermal administration) of the composition or the pharmaceutical composition across a mucosal or skin barrier of a subject to prevent, ameliorate, or treat a disease or disorder. In some embodiments, the kit comprises a medicament comprising the composition or the pharmaceutical composition, and a package insert comprising instructions for administration (e.g., nasal, ocular, or dermal administration) of the medicament across a mucosal or skin barrier a subject to prevent, ameliorate, or treat a disease or disorder.

### EXAMPLES

### Methods and Materials

### Materials

All bile salts and reagents (PBS, Ovalbumin, Bovine Serum Albumin) were purchased from either Millipore Sigma (Billerica, MA) or Santa Cruz Biotechnologies (Dallas, TX). Porcine gastrointestinal tracts were obtained from local farms immediately following sacrifice and kept on ice to preserve tissue pending further dissection. Human insulin and Vancomycin hydrochloride were purchased from Millipore Sigma, Calcitonin from human was purchased from Synnovator (Durham, NC), and Semaglutide was purchased from Proactive Molecular Research (Alachua, FL).

### Formulation Preparation

Formulations were prepared by mixing 9 parts of excipient solution, prepared in 100 mM phosphate-buffered saline solution (PBS) at 1.1× final intended concentration, with 1 part of peptide stock solution (at 10× final intended concentration). Vancomycin hydrochloride stock solution was prepared in PBS at 20 mg/mL. Semaglutide hydrochloride stock solution was prepared in PBS at 5 mg/mL. Insulin stock solution was prepared in 2% Acetic Acid solution at 1 mg/mL. Calcitonin stock solution was prepared in ultrapure water at 1 mg/mL.

### Intestinal Permeation Assay

Permeation studies were performed using a porcine ex vivo intestinal tissue assay described in von Erlach, et al. (2020) Nat Biomed Eng 4:544-559; US Publication No. 2019/0064153; and US Publication No. 2021/0106525, each of which is incorporated herein by reference. The assay is referred to as the GI Tract-Organ Robotic Interface System (GI-ORIS) in the Examples section below.

As previously described, this assay enables the quantification of apical to basolateral transport across fully intact porcine-derived gastrointestinal tissue with high oral human drug absorption predictability. A custom device confines larger tissue explants into individual compartments with donor and receiver chambers that face the apical and basolateral faces of the intestinal tissue, respectively. Porcine tissue was dissected as previously described from freshly harvested gastrointestinal tracts and jejunal segments were stored in calcium-free Krebs buffer at 4° C. for 16 hours prior to mounting between the receiver and donor compartments of a GI-ORIS device. Receiver compartments were filled with 150 μL of either 5% Bovine Serum Albumin (for Vancomycin) or 0.1% Ovalbumin (for Calcitonin, Insulin, Semaglutide) solutions prior to tissue mounting. A 50 μL aliquot of each formulation was applied to each donor chamber, followed by sealing of the donor chamber and incubation at room temperature for 4 hours. A 80 μL aliquot was subsequently collected from each receiver chamber and stored at −80° C. for downstream analysis of peptide concentration.

### Analytical Methods

Vancomycin in receiver fluid samples was detected using LC-TOF. Vancomycin samples were diluted with acetonitrile, centrifuged, and the supernatant collected and further diluted with ultrapure water, followed by injection into LC-TOF. Quantification was performed relative to standard curves prepared in receiver fluid with no exposure to peptide.

Semaglutide in receiver fluid samples was detected using LC-QQQ. Semaglutide samples were processed via solid-phase extraction, followed by elution in a mixture of acetonitrile and ultrapure water, concentration, and injection into LC-QQQ. Quantification was performed relative to standard curves prepared in receiver fluid with no exposure to peptide.

Calcitonin and Insulin in receiver fluid samples (following a 1000× dilution in PBS) were detected using established protocols of commercial sandwich ELISA kits (Abcam, Waltham, MA, and Thermo Fisher Scientific, Waltham, MA). TMB chromogen absorbance was converted to peptide concentration relative to a standard curve prepared using provided standards in ELISA buffer.

Peptide permeability was calculated using the following expression: % Pm=300* (Measured receiver concentration/Initial donor concentration). Fold change peptide permeability relative to buffer control was calculated by dividing % Pm (formulation) by % Pm (un-formulated, PBS). Adjusted log permeability was calculated by taking the logarithm of peptide permeability for each formulation and applying a correction factor to all log-transformed permeability data based on the performance of 3-6 benchmark formulations relative to their historical performance in order to normalize inter-experiment tissue permeability variance.

### Example 1: In Vitro Screening of Intestinal Permeability of Polypeptide Formulations Containing a Bile Salt or Bile Salt Combination

Intestinal permeation was evaluated for therapeutic polypeptides formulated in combination with one or more bile salts using the GI-ORIS. This system is described in detail in U.S. Patent Publication No. 2019/0064153 and also described in the Materials and Methods section. The GI-ORIS relies on custom designed plates that confine porcine GI tissue in sealed wells by magnetic compression. The formulations were evaluated using the GI-ORIS, rather than a traditional Caco-2 transwell assay, as it has been demonstrated to provide higher predictive capacity for human oral drug absorption (see, e.g., von Erlach, 2020). Additionally, whereas a traditional Caco-2 transwell assay is typically limited to testing lower excipient concentrations due to loss of barrier integrity and concomitant cytotoxicity, the GI-ORIS allows for evaluation of formulations containing high concentrations of bile acids (e.g., concentrations exceeding the CMC).

The therapeutic polypeptides selected for evaluation are identified in Table 1. The large size of the polypeptides (i.e., having a molecular weight greater than about 1 kDa) contributes to their poor absorption in the small intestine. It was investigated whether formulation with one or more bile acids would improve their permeation through intestinal tissue (“intestinal perfusion”). It was reasoned that enhanced intestinal perfusion demonstrated by GI-ORIS would translate to enhanced systemic bioavailability when orally administering the formulation in vivo.

TABLE 1
Therapeutic Polypeptides

| | Molecular | |
|---|---|---|
| | Weight | |
| Polypeptide | (Daltons) | Commercial Supplier |
| Human Calcitonin | 3,417 | Custom-synthesized by Synnovator |
| | | (Durham, NC) |
| Human Insulin | 11,981 | Millipore Sigma Catalog No. 9011 |
| Semaglutide | 4,113 | Custom-synthesized by Proactive |
| | | Molecular Research (Alachua, FL) |
| Vancomycin | 1,449 | Millipore Sigma Catalog No. |
| hydrochloride | | SBR00001 |

The bile acids tested in the formulations are identified in Table 2. The bile acids selected for testing vary in the number of hydroxyl groups, stereochemistry of hydroxyl groups, and conjugation at terminal acid. Approximate values for the critical micellular concentration (CMC) provided in Table 2 are based on methods described in Natalini, et al J Pharm Biomed Anal. (2014) 87:62-81.

TABLE 2
Bile Acids

| Bile | | | | CMC | Com- |
|---|---|---|---|---|---|
| Acid | | | CMC | (mg/ | mercial |
| Name | Acronym | Structure | (mM) | mL) | Source |
| Sodium tauro- cholate | STC | 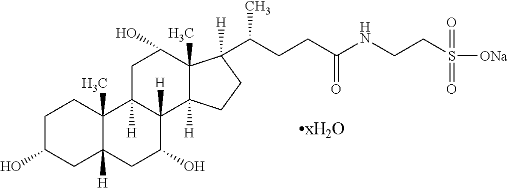 | 6 mM | 3.2 mg/ ml | Sigma 86339 |
| Sodium glyco- cholate | GCA | 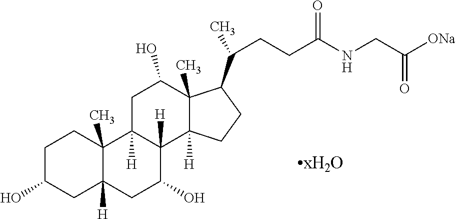 | 11 mM | 5.4 mg/ mL | Sigma G7132 |
| Sodium cholate | CHA | 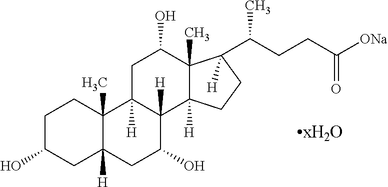 | 11 mM | 4.5 mg/ mL | Sigma S1702 |
| 3-[(3- Chol- amido- propyl) dimethyl- ammo- nio]-1- propane- sulfonate | CHAPS | 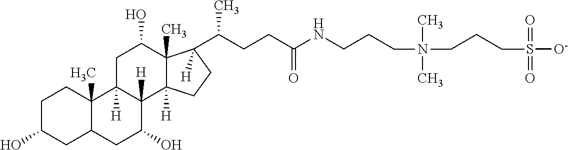 | | | Sigma C3023 |
| Sodium tauro- deoxy- cholate | TDCA | 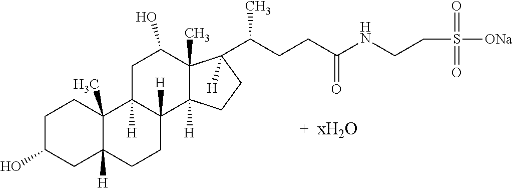 | 3 mM | 1.6 mg/ mL | Santa Cruz Sc- 253596 |
| Sodium glyco- deoxy- cholate | GDCA | 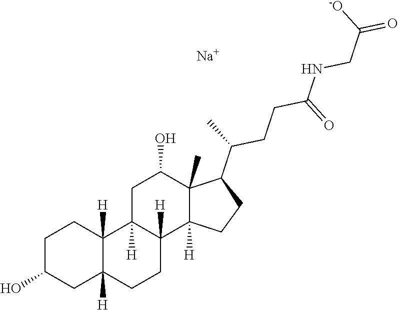 | 2 mM | 0.9 mg/ mL | Santa Cruz Sc- 280755 |
| Sodium deoxy- cholate | DCA | 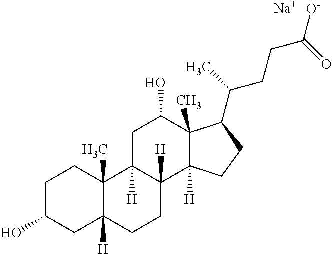 | 5 mM | 2.0 mg/ ml | Sigma D6750 |
| Sodium tauro- cheno- deoxy- cholate | TCDCA | 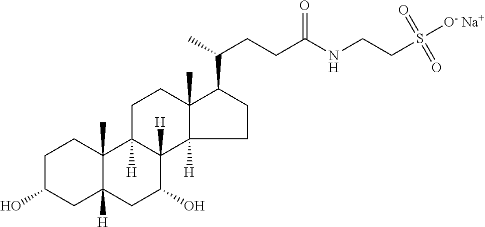 | 4 mM | 2.1 mg/ mL | Santa Cruz Sc- 281162 |
| Sodium glyco- cheno- deoxy- cholate | GCDCA | 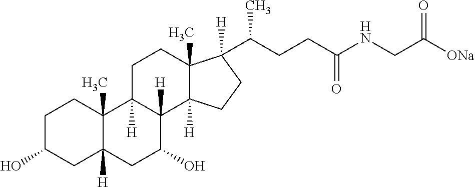 | 2 mM | 0.9 mg/ mL | Santa Cruz. Sc- 207722 |
| Sodium cheno- deoxy- cholate | CDCA | 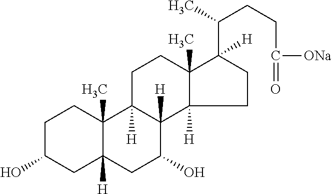 | 4 mM | 1.6 mg/ mL | Santa Cruz Sc- 280637 |

“CMC” = Critical micellular concentration

The formulations were prepared in 100 mM phosphate buffered saline (PBS) pH 7.4 and contained (i) human calcitonin at 100 μg/mL; human insulin at 100 μg/mL; semaglutide at 1 mg/mL; or vancomycin at 2 mg/mL; and (ii) a bile acid or bile acid combination selected from Table 3 at the indicated concentration.

TABLE 3
Bile Acid (BA) or BA Combination and Concentration Thereof (mg/mL)
Present in Formulations Evaluated for Intestinal Permeability

| | | BA 1 | | BA 2 | | BA 3 | | BA 4 |
|---|---|---|---|---|---|---|---|---|
| Name | BA 1 | (mg/mL) | BA 2 | (mg/mL) | BA 3 | (mg/mL) | BA4 | (mg/mL) |
| STC | STC | 128 | — | — | — | — | — | — |
| GCA | GCA | 128 | — | — | — | — | — | — |
| CHA | CHA | 128 | — | — | — | — | — | — |
| CHAPPS | CHAPPS | 128 | — | — | — | — | — | — |
| TDCA | TDCA | 128 | — | — | — | — | — | — |
| GDCA | GDCA | 128 | — | — | — | — | — | — |
| DCA | DCA | 128 | — | — | — | — | — | — |
| TCDCA | TCDCA | 128 | — | — | — | — | — | — |
| GCDCA | GCDCA | 128 | — | — | — | — | — | — |
| GCDCA | GCDCA | 128 | — | — | — | — | — | — |
| CDCA | CDCA | 128 | — | — | — | — | — | — |
| 2BSGCA | STC | 64 | GDCA | 64 | — | — | — | — |
| 2BSSTC | GCA | 64 | TDCA | 64 | — | — | — | — |
| 4BS | STC | 32 | GCA | 32 | GDCA | 32 | TDCA | 32 |

The methods section describes the approach taken to measure the quantity of therapeutic polypeptide that perfused through the GI tissue using the GI-ORIS. The fold change in peptide permeability for the formulated peptide compared to an unformulated buffer control was used to evaluate enhancement in permeability as a result of formulation with the one or more bile acids (see Materials and Methods section for description of the peptide permeability and fold change calculations). For each formulation of calcitonin, insulin, semaglutide, or vancomycin, the fold change in permeability was averaged over six replicates measured in a single experiment.

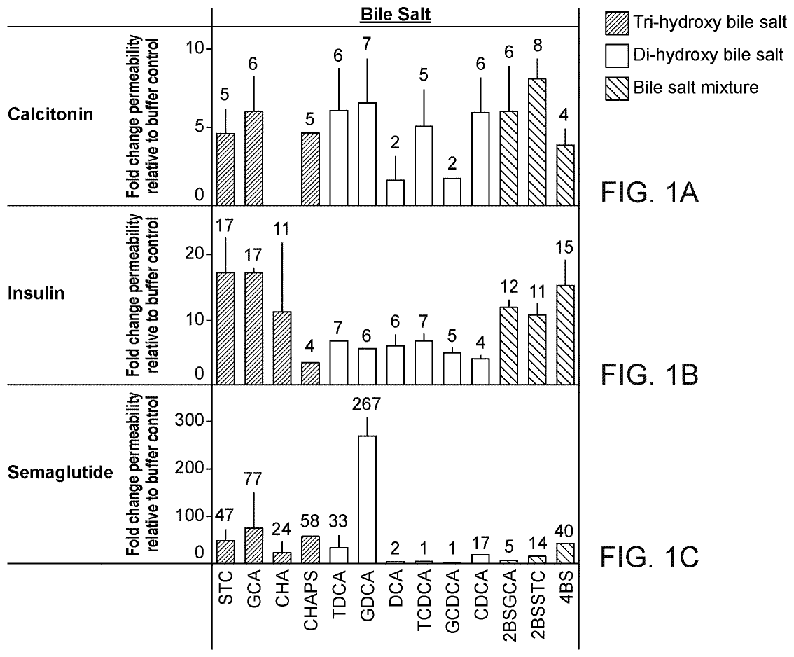

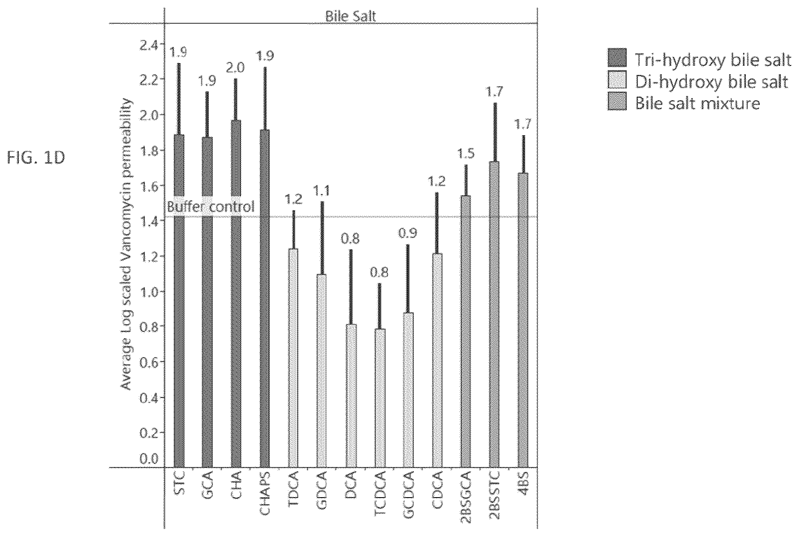

As shown in FIGS. 1A-1D, formulation with a bile salt improved the intestinal permeability for each of the tested peptides. The maximum fold enhancement over buffer control ranged from 5 to 267 depending on the peptide. Generally, it was observed that tri-hydroxy bile salts (GCA, STC, CHA, CHAPS) provided the greatest enhancement in intestinal permeability.

Together, these results demonstrate formulation of a low permeability polypeptide with one or more bile acids enhances the intestinal permeability of the polypeptide.

### Example 2: In Vitro Screening of Intestinal Permeability of Vancomycin Formulations Containing GCA or STC

This Example describes use of the GI-ORIS to evaluate intestinal permeability of formulations containing vancomycin and various concentrations of a bile acid that was sodium glycocholate (GCA) or sodium taurocholate (STC). The methods section describes the approach taken to measure the quantity of vancomycin that perfused through the GI tissue. The fold change in vancomycin permeability for formulated vancomycin compared to unformulated vancomycin in buffer was used to evaluate enhancement in permeability as a result of formulation with the bile acid (see Materials and Methods section for description of the peptide permeability and fold change calculations). The logarithm of vancomycin permeability for the formulation was normalized to account for inter-experiment tissue permeability variance and averaged over 6-30 replicates measured in 1-5 experiments as described in the Materials and Methods section.

Formulations were prepared in 100 mM PBS pH 7.4 and contained (i) vancomycin at 2 mg/mL; and (ii) GCA at a concentration identified in Table 4 or STC at a concentration identified in Table 5.

TABLE 4
Vancomycin Formulated with GCA Alone

| Formulation | Excipient Concentration |
|---|---|
| GCA_32 | 32 mg/mL Sodium Glycocholate |
| GCA_80 | 80 mg/mL Sodium Glycocholate |
| GCA_100 | 100 mg/mL Sodium Glycocholate |
| GCA_128 | 128 mg/mL Sodium Glycocholate |
| GCA_160 | 160 mg/mL Sodium Glycocholate |

TABLE 5
Vancomycin Formulated with STC Alone

| Formulation | Excipient Concentration |
|---|---|
| STC_32 | 32 mg/mL Sodium Taurocholate |
| STC_128 | 128 mg/mL Sodium Taurocholate |
| STC_160 | 160 mg/mL Sodium Taurocholate |

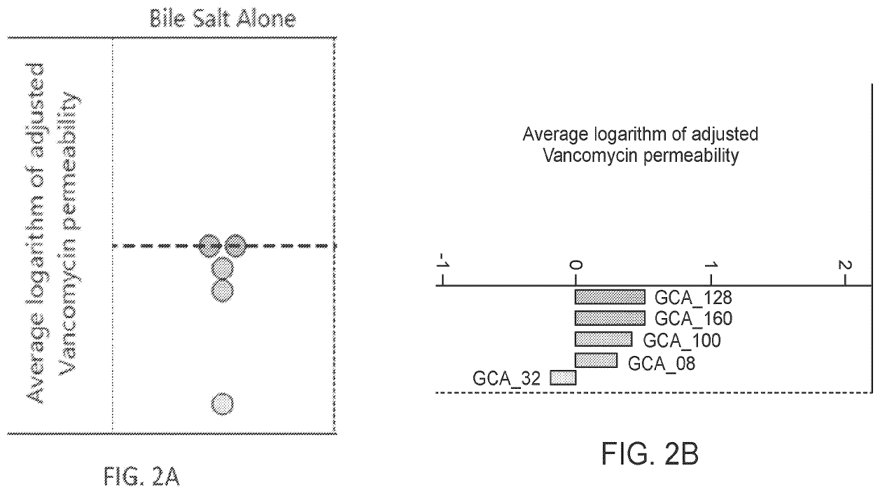

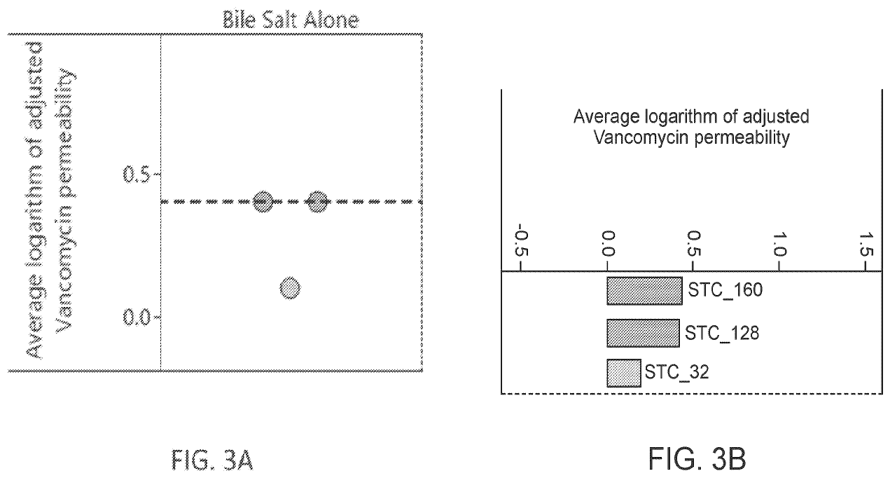

The average logarithm of inter-experiment scaled vancomycin permeability for formulations containing GCA alone are shown in FIGS. 2A-2B and for formulations containing STC alone are shown in FIGS. 3A-3B. Together these results demonstrate that formulations of vancomycin with a bile salt (e.g., at a concentration of about 128 mg/mL) result in improved peptide permeability.

### Example 3: In Vitro Screening of Vancomycin Intestinal Permeability for Formulations Containing a Bile Salt and a Co-Excipient

This Example describes use of the GI-ORIS to evaluate intestinal permeability of formulations containing vancomycin, a bile acid, and a co-excipient. It was evaluated whether intestinal permeability was improved for vancomycin formulated with both the bile acid and co-excipient as compared to vancomycin formulated with the bile acid alone. The bile salt selected for testing was sodium glycocholate (GCA) or sodium taurocholate (STC). The co-excipient was selected from a diverse set of chemical classes, including dendrimers and polymers, chelators, fillers, and mucoadhesives.

The methods section describes the approach taken to measure the quantity of vancomycin that perfused through the GI tissue. The fold change in permeability for formulated vancomycin compared to unformulated vancomycin in buffer was used to evaluate enhancement in permeability as a result of formulation with the bile acid alone or bile acid and co-excipient (see Materials and Methods section for description of the peptide permeability and fold change calculations). The logarithm of vancomycin permeability for each formulation was normalized to account for inter-experiment tissue permeability variance and averaged over 6-30 replicates measured in 1-5 experiments as described in the Materials and Methods section.

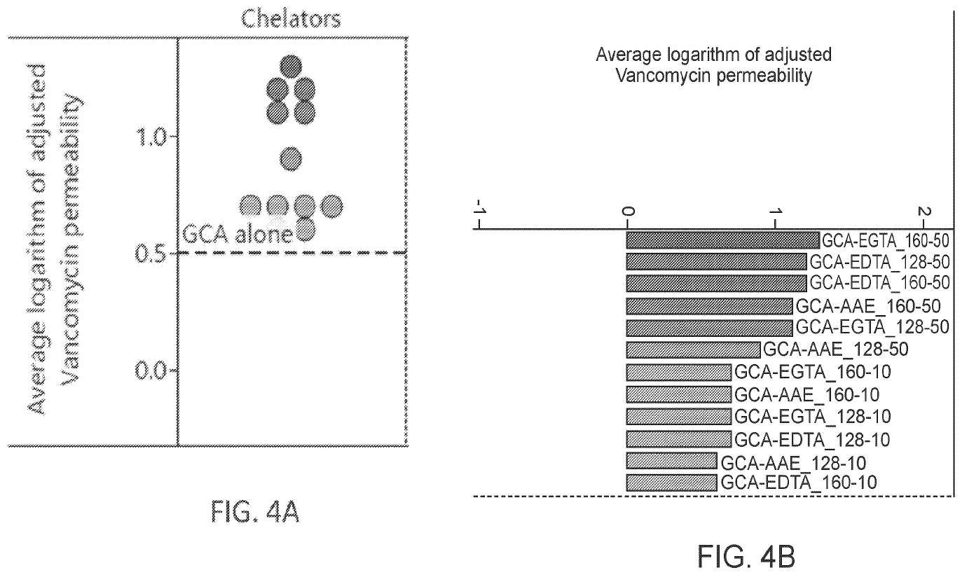

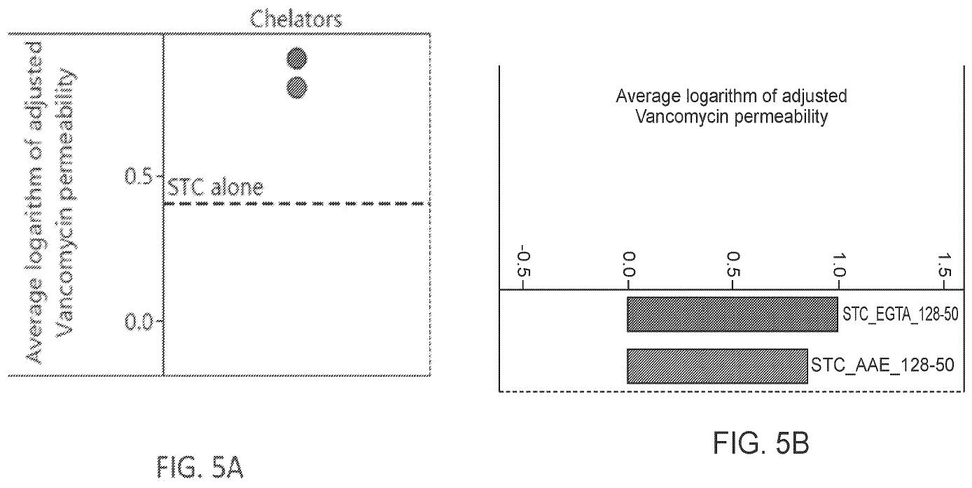

Formulations containing vancomycin, a bile salt (GCA or STC), and a chelating agent (EGTA, EDTA, or AAE) were evaluated. The formulations were prepared in 100 mM PBS pH 7.4 and contained (i) vancomycin at 2 mg/mL; and (ii)(a) GCA and chelator, each at concentrations identified in Table 6; or (b) STC and chelator, each at concentrations identified in Table 7. The average logarithm of inter-experiment scaled vancomycin permeability for formulations containing GCA and chelator are shown in FIGS. 4A-4B and for formulations containing STC and chelator are shown in FIGS. 5A-5B. Comparison is made respectively to vancomycin formulations containing GCA alone or containing STC alone. These results demonstrate that vancomycin formulations containing both a bile salt and a chelating agent had improved intestinal permeability compared to vancomycin formulations with bile salt alone.

TABLE 6
Vancomycin Formulated with GCA and a Chelating Agent

| Formulation | Excipient Concentration |
|---|---|
| GCA-EGTA_128-10 | 128 mg/mL GCA; 10 mg/mL EGTA |
| GCA-EGTA_128-50 | 128 mg/mL GCA; 50 mg/mL EGTA |
| GCA-EGTA_160-10 | 160 mg/mL GCA; 10 mg/mL EGTA |
| GCA-EGTA_160-50 | 160 mg/mL GCA; 50 mg/mL EGTA |
| GCA-EDTA_128-10 | 128 mg/mL GCA; 10 mg/mL EDTA |
| GCA-EDTA_128-50 | 128 mg/mL GCA; 50 mg/mL EDTA |
| GCA-EDTA_160-50 | 160 mg/mL GCA; 50 mg/mL EDTA |
| GCA-AAE_128-50 | 128 mg/mL GCA; 50 mg/mL AAE |
| GCA-AAE_160-10 | 160 mg/mL GCA; 10 mg/mL AAE |
| GCA-AAE_160-50 | 160 mg/mL GCA; 50 mg/mL AAE |

Sodium Glycocholate (GCA);
Sodium Ethyleneglycol-bis(β-aminoethyl)-N,N,N′,N′-tetraacetate (EGTA);
Sodium Ethylenedinitrilotetraacetate (EDTA);
Sodium Glutamate (AAE)

TABLE 7
Vancomycin Formulated with STC and a Chelating Agent

| Formulation | Excipient Concentration |
|---|---|
| STC-EGTA_128-50 | 128 mg/mL STC; 50 mg/mL EGTA |
| STC-AAE_128-50 | 128 mg/mL STC; 50 mg/mL AAE |

Sodium Taurocholate (STC);
Sodium Ethyleneglycol-bis(β-aminoethyl)-N,N,N′,N′-tetraacetate (EGTA);
Sodium Glutamate (AAE)

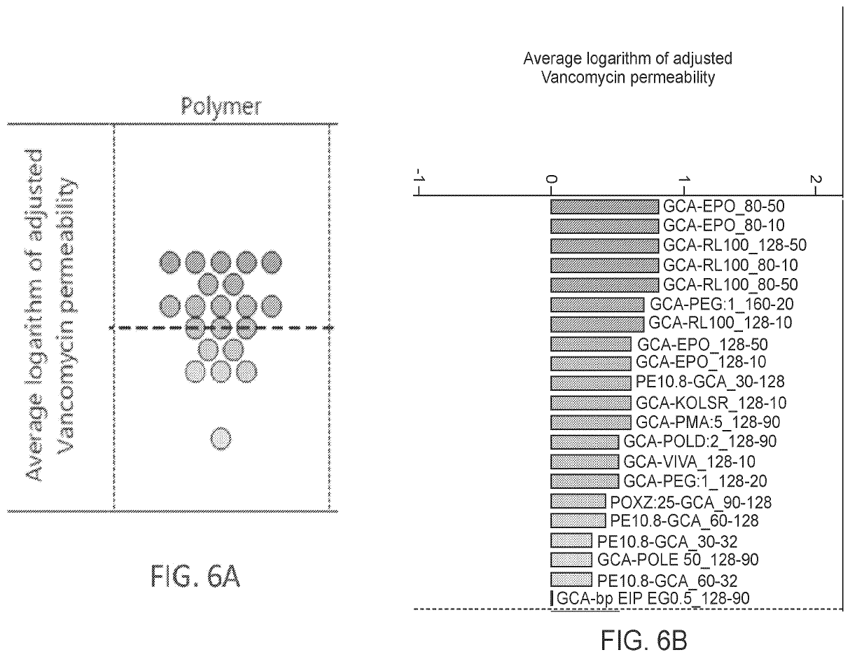

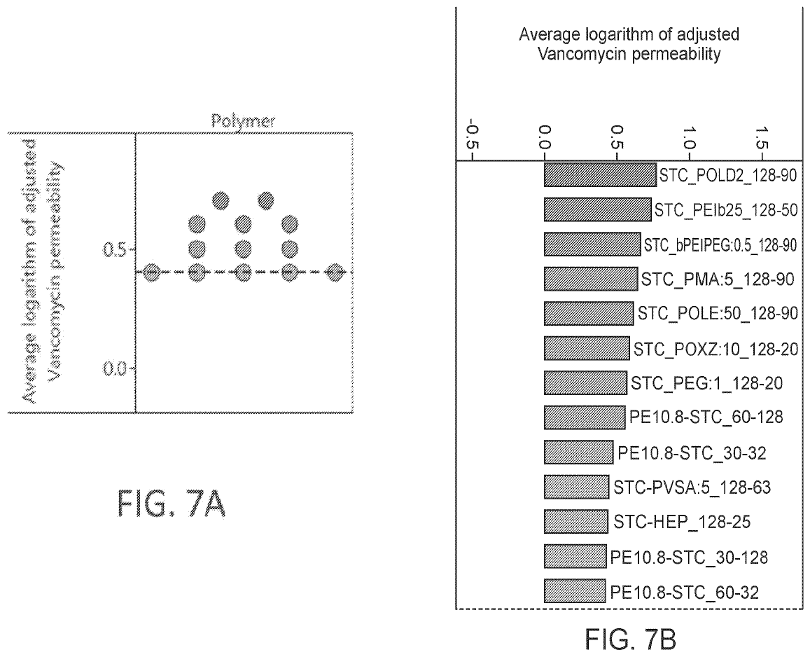

Formulations containing vancomycin, a bile acid, and a co-excipient that was a polymer were evaluated. The formulations were prepared in 100 mMPBS pH 7.4 and contained (i) vancomycin at 2 mg/mL; and (ii)(a) GCA and polymer, each at concentrations identified in Table 8; or (b) SIC and polymer, each at concentrations identified in Table 9. The average logarithm of inter-experiment scaled vancomycin permeability for formulations containing GCA and polymer are shown in FIGS. 6A-6B and for formulations containing SIC and polymer are shown in FIGS. 7A-7B. Comparison is made respectively to vancomycin formulations containing GCA alone or containing SIC alone. These results demonstrate certain combinations of polymer and bile salt enhance vancomycin permeability compared to formulation with bile salt alone.

TABLE 8
Vancomycin Formulated with GCA and a Polymer

| Formulation | Excipient Concentration |
|---|---|
| GCA-EPO_80-10 | 80 mg/mL GCA; 10 mg/mL EPO |
| GCA-EPO_80-50 | 80 mg/mL GCA; 50 mg/mL EPO |
| GCA-EPO_128-10 | 128 mg/mL GCA; 10 mg/mL EPO |
| GCA-EPO_128-50 | 128 mg/mL GCA; 50 mg/mL EPO |
| GCA-RL100_80-10 | 80 mg/mL GCA; 10 mg/mL RL100 |
| GCA-RL100_80-50 | 80 mg/mL GCA; 50 mg/mL RL100 |
| GCA-RL100_128-10 | 128 mg/mL GCA; 10 mg/mL RL100 |
| GCA-RL100_128-50 | 128 mg/mL GCA; 50 mg/mL RL100 |
| GCA-PEG:1_128-20 | 128 mg/mL GCA; 20 mg/mL PEG-1kDa |
| GCA-PEG:1_160-20 | 160 mg/mL GCA; 20 mg/mL PEG-1kDa |
| PEI:b0.8-GCA_30-32 | 32 mg/mL GCA; 30 mg/mL PEI:b0.8 |
| PEI:b0.8-GCA_30-128 | 128 mg/mL GCA; 30 mg/mL PEI:b0.8 |
| PEI:b0.8-GCA_60-32 | 32 mg/mL GCA; 60 mg/mL PEI:b0.8 |
| GCA-KOLSR_128-10 | 128 mg/mL GCA; 10 mg/mL KOLSR |
| GCA-PMA:5_128-90 | 128 mg/mL GCA; 90 mg/mL PMA:5 |
| GCA-POLD:2_128-90 | 128 mg/mL GCA; 90 mg/mL POLD:2 |
| GCA-VIVA_128-10 | 128 mg/mL GCA; 10 mg/mL VIVA |
| POXZ:25-GCA_90-128 | 128 mg/mL GCA; 90 mg/mL POXZ:25 |
| GCA-POLE:50_128-90 | 128 mg/mL GCA; 90 mg/mL POLE:50 |
| GCA-bPEIPEG:0.5_128-90 | 128 mg/mL GCA; 90 mg/mL bPEIPPEG:0.5 |
| PE10.8-GCA 60-128 | 128 mg/mL GCA, 60 mg/mL PEI:b0.8 |

Sodium Glycocholate (GCA);
Eudragit E PO (EPO);
Eudragit RL 100 (RL100);
Poly(ethylene glycol) 1 kDa (PEG-1kDa);
Branched Poly(ethyleneimine) 800 Da (PEI:b0.8);
Kollidon SR (KOLSR);
Poly(methacrylic acid) 5 kDa (PMA:5);
Poly(aspartic acid) 2 kDa (POLD:2);
Poly(2-ethyloxazoline) 25 kDa (POXZ:25);
Poly(glutamic acid) 50 kDa (POLE:50);
Branched Poly(ethyleneimine)-co-Poly(ethylene glycol) 500 Da (bPEIPPEG:0.5);
Vivacoat (VIVA)

TABLE 9
Vancomycin Formulated with STC and a Polymer

| Formulation | Excipient Concentration |
|---|---|
| STC-POLD:2_128-90 | 128 mg/mL STC; 90 mg/mL POLD:2 |
| STC-PEI:B:25_128-50 | 128 mg/mL STC; 50 mg/mL PEI:b25 |
| STC-bPEIPEG:0.5_128-90 | 128 mg/mL STC; 90 mg/mL bPEIPEG:0.5 |
| STC-PMA:5_128-90 | 128 mg/mL STC; 90 mg/mL PMA:5 |
| STC-POLE:50_128-90 | 128 mg/mL STC; 90 mg/mL POLE:50 |
| STC:POXZ:10_128-20 | 128 mg/mL STC; 20 mg/mL POXZ:10 |
| STC-PEG:1_128-20 | 128 mg/mL STC; 20 mg/mL PEG:1 |
| PE10.8-STC_60-128 | 128 mg/mL STC; 60 mg/mL PEI:b0.8 |
| PE10.8-STC_30-32 | 32 mg/mL STC; 32 mg/mL PEI:b0.8 |
| STC-PVSA:5_128-63 | 128 mg/mL STC; 63 mg/mL PVSA:5 |
| STC-HEP_128-25 | 128 mg/mL STC; 25 mg/mL HEP |
| PE10.8-STC_30-128 | 128 mg/mL STC; 30 mg/mL PEI:b0.8 |
| PE10.8-STC_60-32 | 32 mg/mL STC; 60 mg/mL PEI:b0.8 |

Sodium Taurocholate (STC);
Poly(aspartic acid) 2 kDa (POLD:2);
Branched poly(ethyleneimine) 25 kDa (PEI:b25);
Poly(methacrylic acid) 5 kDa (PMA:5);
Poly(glutamic acid) 50 kDa (POLE:50);
Poly(2-ethyloxazoline) 10 kDa (POXZ:10);
Poly(ethylene glycol) 1 kDa (PEG:1);
Branched poly(ethyleneimine) 800 Da (PEI:b0.8);
Poly(vinylsulfonic acid) 5 kDa (PVSA:5);
Heparin sodium (HEP)

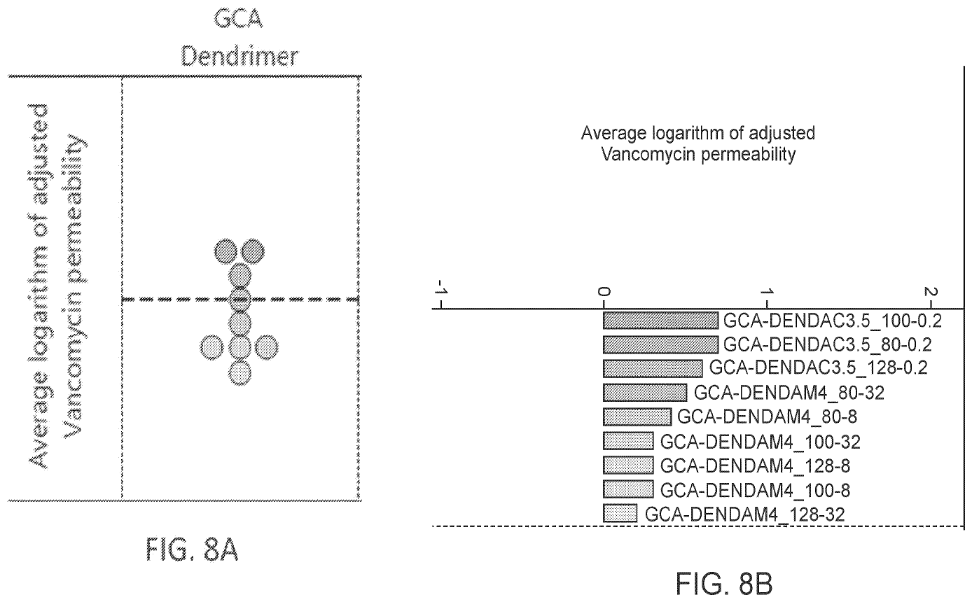

Formulations containing vancomycin, a bile acid, and a co-excipient that was a dendrimer were evaluated. The formulations were prepared in 100 mM PBS pH 7.4 and contained (i) vancomycin at 2 mg/mL; and (ii) GCA and dendrimer, each at concentrations identified in Table 10. The formulations were prepared in 100 mM phosphate buffered saline (PBS) pH 7.4. The average logarithm of inter-experiment scaled vancomycin permeability for formulations containing GCA and dendrimer are shown in FIGS. 8A-8B, with comparison made to vancomycin formulations containing GCA alone. These results demonstrate certain combinations of dendrimer and bile salt enhance vancomycin permeability compared to formulation with bile salt alone.

TABLE 10
Vancomycin Formulated with GCA and a Dendrimer

| Formulation | Excipient Concentration |
|---|---|
| GCA-DENDAC3.5_80-0.2 | 80 mg/mL GCA; 0.2 mg/mL DENDAC3.5 |
| GCA-DENDAC3.5_100-0.2 | 100 mg/mL GCA; 0.2 mg/mL DENDAC3.5 |
| GCA-DENDAC3.5_128-0.2 | 128 mg/mL GCA; 0.2 mg/mL DENDAC3.5 |
| GCA-DENDAM4_80-8 | 80 mg/mL GCA; 8 mg/mL DENDAM4 |
| GCA-DENDAM4_80-32 | 32 mg/mL GCA; 32 mg/mL DENDAM4 |
| GCA-DENDAM4_100-8 | 100 mg/mL GCA; 8 mg/mL DENDAM4 |
| GCA-DENDAM4_100-32 | 32 mg/mL GCA; 32 mg/mL DENDAM4 |
| GCA-DENDAM4_128-8 | 128 mg/mL GCA; 8 mg/mL DENDAM4 |
| GCA-DENDAM4_128-32 | 128 mg/mL GCA; 32 mg/mL DENDAM4 |

Sodium Glycocholate (GCA);
Carboxylate terminal Poly(amidoamine) dendrimer generation 3.5 (DENDAC3.5);
Amine terminal Poly(amidoamine) dendrimer generation 4 (DENDAM4)

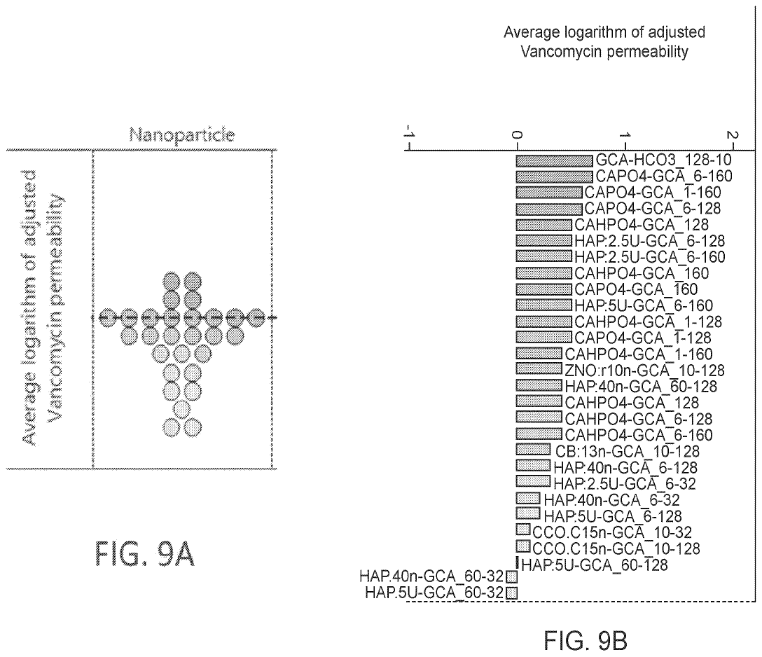

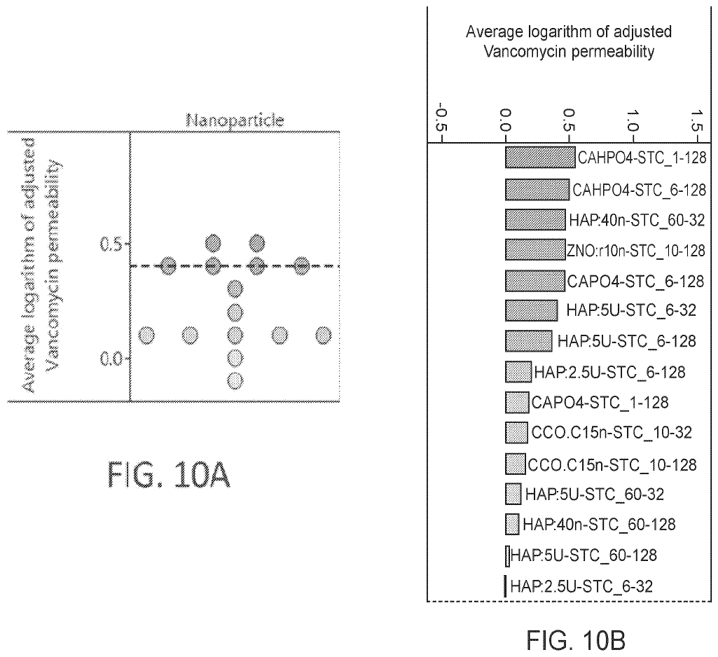

Formulations containing vancomycin, a bile acid, and a co-excipient that was a nanoparticle were prepared with (i) vancomycin at 2 mg/mL; and (ii)(a) GCA and nanoparticle, each at concentrations identified in Table 11; or (b) STC and a nanoparticle, each at concentrations identified in Table 12. The formulations were prepared in 100 mM phosphate buffered saline (PBS) pH 7.4. The average logarithm of inter-experiment scaled vancomycin permeability for formulations containing GCA and nanoparticle are shown in FIGS. 9A-9B and for formulations containing STC and nanoparticle are shown in FIGS. 10A-10B. Comparison is made respectively to vancomycin formulations containing GCA alone or containing STC alone. Nanoparticles have advantageous properties for drug formulation (e.g., as fillers or mucoadhesives). These results demonstrate certain nanoparticles may be included in a drug formulation containing a bile salt without negatively impacting permeability of the drug compared to formulation with the bile salt alone.

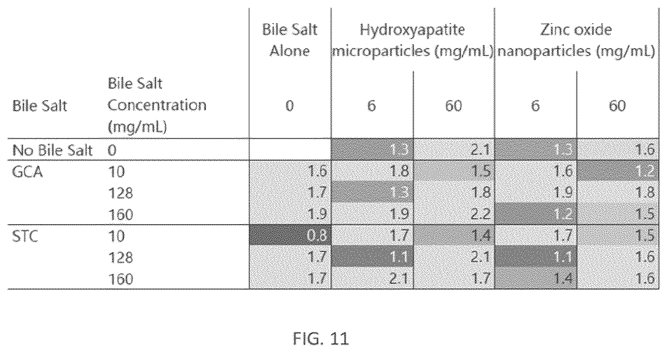

Shown in FIG. 11 is a two-dimensional heat map comparing the average logarithm of inter-experiment scaled vancomycin permeability for formulations containing different concentrations of a bile salt (GCA or STC) alone or bile salt (GCA or STC) and a bioceramic nanoparticle (hydroxyapatite microparticles or zinc oxide nanoparticles) as compared to unformulated vancomycin. Vancomycin permeability was aggregated over 2-4 experiments with 12-24 total replicates. Cells with lighter shading represent formulations having increased vancomycin permeability. These results demonstrate inclusion of the bioceramic nanoparticle does not negatively impact permeability compared to formulation of vancomycin with bile salt alone.

TABLE 11
Vancomycin Formulated with GCA and a Nanoparticle

| Formulation | Excipient Concentration |
|---|---|
| GCA-HCO3_128-10 | 128 mg/mL GCA; 10 mg/mL HCO3 |
| CAPO4-GCA_1-128 | 128 mg/mL GCA; 1 mg/mL CAPO4 |
| CAPO4-GCA_1-160 | 160 mg/mL GCA; 1 mg/mL CAPO4 |
| CAPO4-GCA_6-128 | 128 mg/mL GCA; 6 mg/mL CAPO4 |
| CAPO4-GCA_6-160 | 160 mg/mL GCA; 6 mg/mL CAPO4 |
| CAPO4-GCA_10-128 | 128 mg/mL GCA; 10 mg/mL CAPO4 |
| CAPO4-GCA_10-160 | 160 mg/mL GCA; 10 mg/mL CAPO4 |
| CAHPO4-GCA_1-128 | 128 mg/mL GCA; 1 mg/mL CAHPO4 |
| CAHPO4-GCA_1-160 | 160 mg/mL GCA; 1 mg/mL CAHPO4 |
| CAHPO4-GCA_6-128 | 128 mg/mL GCA; 6 mg/mL CAHPO4 |
| CAHPO4-GCA_6-160 | 160 mg/mL GCA; 6 mg/mL CAHPO4 |
| CAHPO4-GCA_10-128 | 128 mg/mL GCA; 6 mg/mL CAHPO4 |
| CAHPO4-GCA_10-160 | 160 mg/mL GCA; 10 mg/mL CAHPO4 |
| HAP:2.5U-GCA_6-32 | 32 mg/mL GCA; 6 mg/mL HAP:2.5U |
| HAP:2.5U-GCA_6-128 | 128 mg/mL GCA; 6 mg/mL HAP:2.5U |
| HAP:2.5U-GCA_6-160 | 160 mg/mL GCA; 6 mg/mL HAP:2.5U |
| HAP:5U-GCA_6-128 | 128 mg/mL GCA; 6 mg/mL HAP:5U |
| HAP:5U-GCA_6-160 | 160 mg/mL GCA; 6 mg/mL HAP:5U |
| ZNO:r10n-GCA_10-128 | 128 mg/mL GCA; 10 mg/mL ZNO:r10n |
| HAP:r40n-GCA_6-32 | 32 mg/mL GCA; 6 mg/mL HAP:r40n |
| HAP:r40n-GCA_6-128 | 128 mg/mL GCA; 6 mg/mL HAP:140n |
| HAP:r40n-GCA_60-128 | 128 mg/mL GCA; 60 mg/mL HAP:r40n |
| CB:_13n-GCA_10-128 | 128 mg/mL GCA; 10 mg/mL CB:13n |

Sodium Glycocholate (GCA);
Sodium Bicarbonate (HCO3);
Calcium Phosphate Tribasic Microparticles (CAPO4);
Calcium Phosphate Dibasic Microparticles (CAHPO4);
5 μm Spherical Hydroxyapatite Microparticles (HAP:5U);
2.5 μm Spherical Hydroxyapatite Microparticles (HAP:2.5U);
Zinc Oxide Microparticles (ZNO:r10n);
Hydroxyapatite Rod Shaped Nanoparticles 40 nm diameter (HAP:r40n);
Carbon Black Nanoparticles 13 nm diameter (CB:13n)

TABLE 12
Vancomycin Formulated with STC and a Nanoparticle

| Formulation | Excipient Concentration |
|---|---|
| CAHPO4-STC_1-128 | 128 mg/mL STC; 1 mg/mL CAHPO4 |
| CAHPO4-STC_6-128 | 128 mg/mL STC; 6 mg/mL CAHPO4 |
| CAPO4-STC_1-128 | 128 mg/mL STC; 1 mg/mL CAPO4 |
| HAP:r40n-STC_60-32 | 32 mg/mL STC; 60 mg/mL HAP:r40n |
| HAP:r40n-STC_60-128 | 128 mg/mL STC; 60 mg/mL HAP:r40n |
| HAP:2.5U-STC_6-32 | 32 mg/mL STC; 6 mg/mL HAP:2.5U |
| HAP:2.5U-STC_6-128 | 128 mg/mL STC; 6 mg/mL HAP:2.5U |
| HAP:5U-STC_6-32 | 32 mg/mL STC; 6 mg/mL HAP:5U |
| HAP:5U-STC_6-128 | 128 mg/mL STC; 6 mg/mL HAP:5U |
| HAP:5U-STC_60-32 | 32 mg/mL STC; 60 mg/mL HAP:5U |
| HAP:5U-STC_60-128 | 128 mg/mL STC; 60 mg/mL HAP:5U |
| ZNO:r10n-STC_10-128 | 128 mg/mL STC; 10 mg/mL ZNO:r10n |
| CAPO4-STC_6-128 | 128 mg/mL STC; 6 mg/mL CAPO4 |
| CCO:C15n-STC_10-32 | 32 mg/mL STC; 10 mg/mL 15 nm CCO:C15n |
| CCO:C15n-STC_10-128 | 128 mg/mL STC; 10 mg/mL 15 nm CCO:C15n |

Sodium Taurocholate (STC);
Calcium Phosphate Dibasic Microparticles (CAHPO4);
Hydroxyapatite Rod Shaped Nanoparticles 40 nm diameter (HAP:r40n);
Zinc Oxide Microparticles (ZNO:r10n);
Calcium Phosphate Tribasic Microparticles (CAPO4);
5.0 μm Spherical Hydroxyapatite Microparticles (HAP:5U);
2.5 μm Spherical Hydroxyapatite Microparticles (HAP:5U);
Spherical Calcium Carbonate Nanoparticles (CCO:C15n)

Together these results demonstrate intestinal permeability of vancomycin is enhanced when formulated with a bile salt and co-excipient, particularly a co-excipients from certain chemical classes such as chelators, dendrimers, and polymers. Indeed, a 0.5-1 log unit improvement in vancomycin permeability was observed for certain formulations containing a bile salt and a chelator as compared to formulation with bile salt alone. Additionally, it was found that formulation of vancomycin with a bile salt could further include co-excipients such as nanoparticles that have other advantageous properties for drug delivery (e.g., fillers, mucoadhesives) without decreasing permeability compared to formulation with bile salt alone. These results demonstrate vancomycin formulations containing bile salt can be modified to include co-excipients with desirable properties without negatively impacting formulation performance.

### Example 4: In Vitro Screening of Intestinal Permeability of Therapeutic Polypeptide Formulations Containing a Bile Salt and a Calcium Chelating Agent

This Example describes use of the GI-ORIS to evaluate intestinal permeability of a therapeutic polypeptide formulated to have different concentrations of a bile acid and chelator. The formulations contained a therapeutic polypeptide that was vancomycin, insulin, semaglutide, or calcitonin; a bile salt that was glycocholate (GCA) or sodium taurocholate (STC); and a calcium chelating agent that was monosodium glutamate (AAE) or egtazic acid tetrasodium (EGTA). Comparison of formulated versus unformulated therapeutic polypeptide was performed to determine the efficacy of particular bile salt and chelator combinations and the concentration of each excipient for improving intestinal permeability of the therapeutic polypeptide.

The methods section describes the approach taken to measure the quantity of therapeutic polypeptide that perfused through the GI tissue. The GI-ORIS enabled measurement of intestinal permeability for therapeutic polypeptide formulated with a bile acid alone (GCA or STC) or bile acid (GCA or STC) and a chelator (AAE, EDTA, or EGTA). The fold change in peptide permeability for the formulated peptide compared to an unformulated buffer control was used to evaluate enhancement in permeability as a result of formulation (see Materials and Methods section for description of the peptide permeability and fold change calculations). For each formulation of calcitonin, insulin, or semaglutide, the fold change in permeability was averaged over six replicates measured in a single experiment. For formulations of vancomycin, the fold change in permeability was averaged over 12-24 replicates measured in 2-4 experiments. The logarithm of vancomycin permeability for each formulation was normalized to account for inter-experiment tissue permeability variance as described in the Materials and Methods section.

Formulations of therapeutic polypeptide, a bile salt (GCA or STC), and a chelator (EGTA or AAE) prepared in 100 mM phosphate buffered saline (PBS) pH 7.4 were evaluated. The formulations that were tested combined
(i)(a) human calcitonin at 100 μg/mL; (b) human insulin at 100 μg/mL; (c) semaglutide at 1 mg/mL; or (d) vancomycin at 2 mg/mL;
(ii)(a) GCA at 10, 128, or 160 mg/mL, or (b) STC at 5, 10, 25, 50, 128, or 160 mg/mL; and
(iii)(a) EGTA at 50 or 100 mg/mL, or (b) AAE at 50 or 100 mg/mL. Comparison was made to formulations containing therapeutic polypeptide and bile salt alone or therapeutic polypeptide and chelator alone.

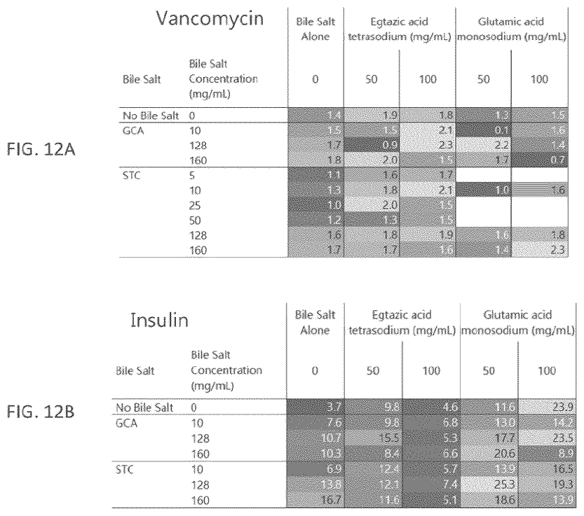

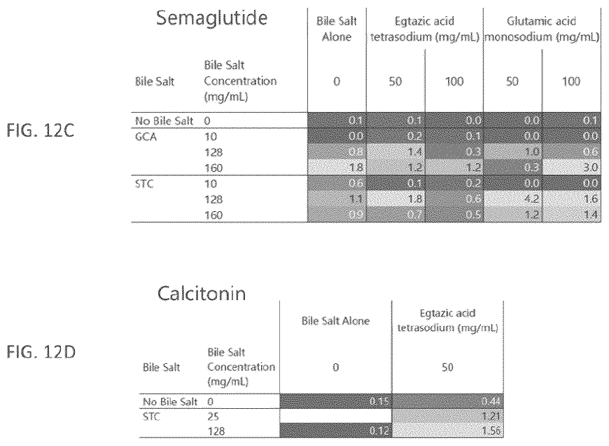

Shown in FIG. 12A is a two-dimensional heat map comparing the average logarithm of inter-experiment scaled vancomycin permeability for formulations containing different concentrations of a bile salt (GCA or STC) alone; chelator (EGTA or AAE) alone; or bile salt (GCA or STC) and chelator (EGTA or AAE) as compared to unformulated vancomycin. Vancomycin permeability was aggregated over 2-4 experiments with 12-24 total replicates. Cells with lighter shading represent formulations having increased vancomycin permeability.

Shown in FIGS. 12B-12D are two-dimensional heat maps comparing the average fold change in insulin permeability (FIG. 12B), semaglutide permeability (FIG. 12C), or calcitonin permeability (FIG. 12D) for formulations containing different concentrations of a bile salt (GCA or STC) alone; chelator (EGTA or AAE) alone; or bile salt (GCA or STC) and chelator (EGTA or AAE) as compared to unformulated buffer control. Cells with lighter shading represent formulations having increased permeability of the therapeutic polypeptide.

Together these results demonstrate enhanced permeability is achieved for combinations of bile salt and calcium chelating agent.

### Example 5: Vancomycin Permeability Enhancement for Formulations Containing a Bile Salt and a Chelator

Having demonstrated vancomycin permeability is improved for formulations containing a bile salt alone or including a chelator compared to unformulated vancomycin, it was further evaluated if this effect is maintained under conditions simulating the dilution that occurs following in vivo administration (i.e., at low excipient concentration). This was done by using GI-ORIS to measure intestinal permeability of vancomycin formulations containing a bile salt (STC) and a chelator, each at concentrations at or below 25 mg/mL.

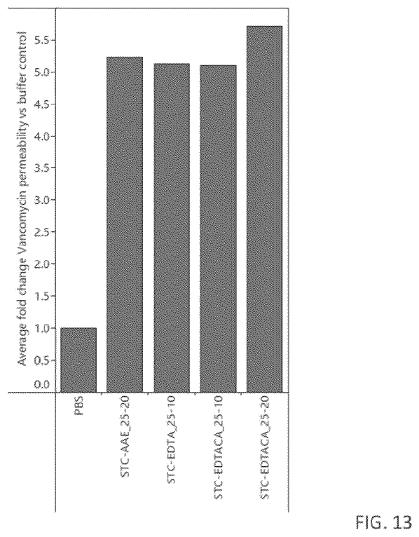

Formulations of vancomycin, a STC, and a chelator (EDTA, AAE, or EDTACA) were evaluated. The formulations were prepared in 100 mM PBS pH 7.4 to contain (i) vancomycin at 2 mg/mL; and (ii) co-excipients at the concentrations listed in Table 13. The average logarithm of inter-experiment scaled vancomycin permeability for formulations containing GCA and chelator are shown in FIG. 13. Comparison was made to unformulated vancomycin.

TABLE 13
Vancomycin Formulated with STC and a Chelator

| Formulation | Excipient Concentration |
|---|---|
| STC-AAE_25-20 | 25 mg/mL STC |
| | 20 mg/mL AAE |
| STC-EDTA_25-10 | 25 mg/mL STC |
| | 10 mg/mL EDTA |
| STC-EDTACA_25-10 | 25 mg/mL STC |
| | 10 mg/mL EDTACA |
| STC-EDTACA_25-20 | 25 mg/mL STC |
| | 20 mg/mL EDTACA |

Sodium Taurocholate (STC);
Ethylenedinitrilotetraacetate (EDTA);
Sodium Glutamate (AAE);
Calcium Disodium Ethylenedinitrilotetraacetate (EDTACA)

Dilution of a formulation occurs following in vivo oral administration. These results demonstrate that even at reduced concentrations of excipient (i.e., 5-25 mg/mL), the enhanced permeability of vancomycin compared to unformulated buffer control is maintained. This data suggests the performance of the formulation following oral administration is not negatively impacted by potential dilution that occurs in vivo.

### Example 6: In Vivo Absorption of Therapeutic Polypeptide Formulations Containing a Bile Salt and a Co-Excipient

Based on the above Examples, it was evaluated whether formulation of a therapeutic polypeptide with a bile salt and an additional co-excipient chelator would result in improved bioavailability in vivo. The therapeutic polypeptides that were evaluated included semaglutide and vancomycin.

The semaglutide formulations were prepared in 100 mM PBS pH 7.4 and contained semaglutide at 1 mg/mL combined with the excipients listed in Table 14. Formulation of semaglutide with sodium caprate (“C10”) represents the benchmark formulation as this excipient is a known peptide permeability enhancer. For example, it has been reported that semaglutide formulated with SNAC (sodium N-(8-[2-hydroxybenzoyl]amino) caprylate) in solid tablet dosage form and administered orally has 1.2% bioavailability in dogs and 0.8% bioavailability in humans (see, e.g., Buckley et al., Sci. Transl. Med. 10, eaar7047 (2018)).

TABLE 14
Semaglutide Formulations

| Formulation | Name | Excipient Concentration |
|---|---|---|
| Benchmark with known | Sodium caprate | 50 mg/mL |
| permeability enhancer | | |
| Bile acid + calcium chelator | STC-EGTA_128-50 | 128 mg/mL STC |
| | | 50 mg/mL EGTA |
| Bile acid + calcium chelator + | SBECD4-STC- | 128 mg/mL STC |
| cyclodextrin | EGTA_90-128-50 | 90 mg/mL SBECD4 |
| | | 50 mg/mL EGTA |

Sodium Taurocholate (STC);
Sodium Ethyleneglycol-bis(β-aminoethyl)-N,N,N′,N′-tetraacetate (EGTA);
Sodium sulfobutylether-b-cyclodextrin (SBECD4)

The vancomycin formulations were prepared in 100 mM PBS pH 7.4 and contained vancomycin at 2 mg/mL combined with the excipients listed in Table 15. Comparison was made to unformulated vancomycin in buffer control. It was evaluated whether vancomycin formulated the indicated excipients would outperform a benchmark reported in the literature, which is 8% vancomycin bioavailability in a similar animal model (i.e., intra-ileum administration in the rat at 20 mg/kg dose; see, e.g., European Patent No. 3,006,045B3).

TABLE 15
Vancomycin Formulations

| Formulation | Name | Excipient Concentration |
|---|---|---|
| Bile acid + calcium chelator | GCA-AAE_128-50 | 128 mg/mL GCA; 50 mg/mL AAE |
| Bile acid + nanoparticle | HAP:5U-GCA_6-160 | 160 mg/mL GCA; 6 mg/mL HAP:5U |
| Bile acid + calcium chelator | STC-EGTA_128-50 | 128 mg/mL STC; 50 mg/mL EGTA |

Sodium Glycocholate (GCA);
Sodium Taurocholate (STC);
Sodium Ethyleneglycol-bis(B-aminoethyl)-N,N,N′,N′-tetraacetate (EGTA);
Sodium Ethylenedinitrilotetraacetate (EDTA);
Sodium Glutamate (AAE);
5 μm Spherical Hydroxyapatite Microparticles (HAP:5U)

Vancomycin and Semaglutide formulations were evaluated in a Sprague Dawley rat intraduodenal pharmacokinetic model to quantify enhancement of intestinal absorption. Anesthetized rats were administered a single bolus dose of formulation (0.5-0.6 mL, 20 mg/kg Vancomycin or Semaglutide) via 22-gauge needle to the duodenum over a 15 second period. Blood samples (300 uL) were collected in K2EDTA tubes by jugular vein sampling prior to dosing and at 0.25, 0.5, 1, 1.5, 2, 4, 6, and 8 hours following administration. The plasma was extracted from the whole blood samples and analyzed by LC-qTOF to quantify Vancomycin or Semaglutide concentration at the respective time point. Pharmacokinetic parameters (Tmax, Cmax, Thalf, AUC0-8, AUCtotal) were calculated by fitting data to a single compartment model.

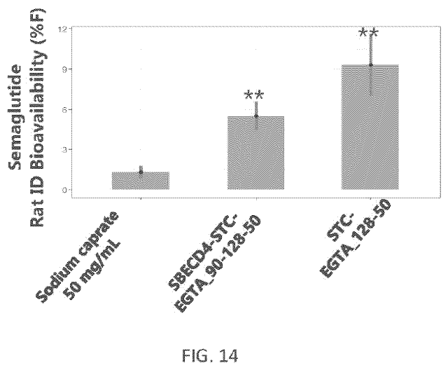

As shown in FIG. 14, semaglutide formulation with a bile salt and chelator had significantly improved bioavailability compared to the benchmark formulation with sodium caprate, which is a well-established permeation enhancer.

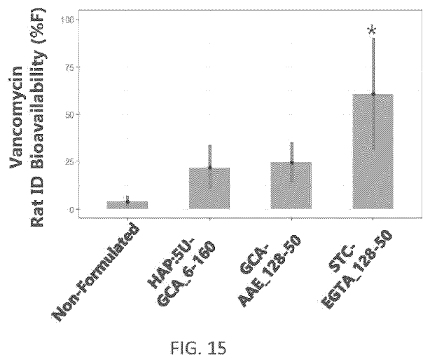

As shown in FIG. 15, vancomycin formulation with a bile salt and chelator (SIC and EGTA) demonstrated significantly improved bioavailability compared to unformulated vancomycin control. The bioavailability observed for this formulation (about 60%) was substantially higher than the benchmark reported in the literature based on vancomycin bioavailability measured in a similar animal model (about 8%).

TABLE 16
Bioavailability evaluation of semaglutide formulations

| Formu- | Avg | SD | Avg | SD | Avg | SD | Avg |
|---|---|---|---|---|---|---|---|
| lation | AUC0-7 | AUC0-7 | Cmax | Cmax | Tmax | Tmax | BA |
| (10 mg/kg) | (ng/mL*h) | (ng/mL*h) | (ng/mL) | (ng/mL) | (hrs) | (hrs) | (%) |
| Sodium | 6613 | 4562 | 1571 | 943 | 0.5 | 0 | 1.29 |
| caprate | | | | | | | |
| 50 mg/mL | | | | | | | |
| STC- | 47854 | 23562 | 10389 | 4786 | 1.3 | 1.2 | 9.30 |
| EGTA_128- | | | | | | | |
| 50 | | | | | | | |
| SBECD4- | 28290 | 10776 | 8039 | 3164 | 0.75 | 0.5 | 5.50 |
| STC- | | | | | | | |
| EGTA_90- | | | | | | | |
| 128-50 | | | | | | | |

TABLE 17
Bioavailability evaluation of vancomycin formulations

| | Avg | SD | Avg | SD | Avg | SD | Avg |
|---|---|---|---|---|---|---|---|
| Formu- | AUCTotal | AUCTotal | Cmax | Cmax | Tmax | Tmax | BA |
| lation | (ng/mL*h) | (ng/mL*h) | (ng/ml) | (ng/ml) | (hrs) | (hrs) | (%) |
| GCA- | 32510 | 28239 | 10911 | 6082 | 0.31 | 0.13 | 24 |
| AAE_128- | | | | | | | |
| 50 | | | | | | | |
| IIAP:5U- | 29150 | 30732 | 12029 | 12526 | 0.50 | 0.35 | 22 |
| GCA_6-160 | | | | | | | |
| Non- | 5015 | 7612 | 2206 | 3500 | 0.63 | 0.60 | 4 |
| Formulated | | | | | | | |
| STC- | 80653 | 78719 | 19817 | 15708 | 0.44 | 0.13 | 61 |
| EGTA_128- | | | | | | | |
| 50 | | | | | | | |

Together these results demonstrate the combination of bile salt and chelator outperforms other bile salt-based formulations in vivo for both vancomycin and semaglutide and support the findings of the GI-ORIS screening.

### Example 7: In Vitro Intestinal Permeability of Antisense Oligonucleotide Formulations Containing a Bile Salt and a Co-Excipient

This Example describes use of the GI-ORIS to evaluate intestinal permeability of formulations containing an antisense oligonucleotide (ASO), a bile acid, and a co-excipient. It was evaluated whether intestinal permeability was improved for an ASO formulated with bile acid (STC or GCA) and co-excipient as compared to ASO formulated with the bile acid alone. Further comparison was made to intestinal permeability of ASO formulated with a known permeability enhancer (sodium caprate or C10) and unformulated ASO in buffer control.

The ASO selected for testing targets human SMAD7 and is under clinical evaluation for treatment of Crohn's disease (Mongersen). Formulations were prepared by mixing 9 parts of excipient solution, prepared in 100 mM phosphate-buffered saline solution (PBS) at 1.1× final intended concentration, with 1 part of mongersen stock solution (at 10× final intended concentration). Mongersen stock solution was prepared in PBS at 3 mg/mL. The formulations contained (i) Mongersen at 0.3 mg/mL; and (ii) a excipient combinations identified in Table 18.

TABLE 18
Mongersen Formulations

| Formulation | Excipient Concentration |
|---|---|
| STC-EGTA_128-50 | 128 mg/mL STC; 50 mg/mL EGTA |
| STC_128 | 128 mg/mL STC |
| GCA-HAP:5U_160-6 | 160 mg/mL GCA; 6 mg/mL HAP:5U |
| STC-EGTA_25-50 | 25 mg/mL STC; 50 mg/mL EGTA |
| GCA_128 | 128 mg/mL GCA |
| GCA-AAE_128-50 | 128 mg/mL GCA; 50 mg/mL AAE |
| STC_EDTACA_25-50 | 25 mg/mL STC; 50 mg/mL EDTACA |
| STC-EDTACA_128-50 | 128 mg/mL STC; 50 mg/mL EDTACA |
| GCA-ZNO:R1ON_160-6 | 160 mg/mL GCA; 6 mg/mL ZNO:r10n |
| C10_50 | 50 mg/mL C10 |

Sodium taurocholate (STC);
Egtazic acid tetrasodium (EGTA);
Sodium glycocholate (GCA);
5 μm Spherical Hydroxyapatite Microparticles (HAP:5U);
Glutamic acid monosodium (AAE);
Calcium disodium edetate (EDTACA)

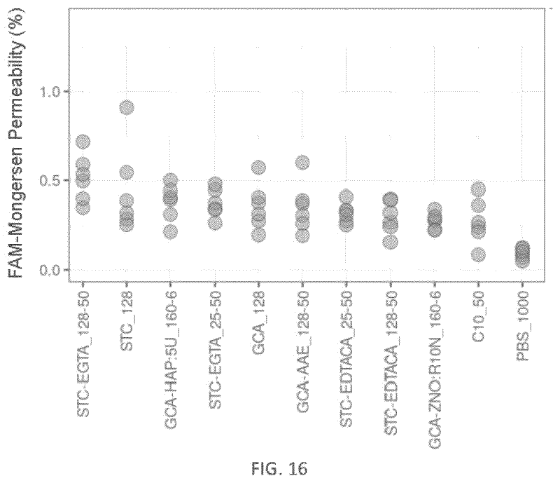

As shown in FIG. 16, formulation of the ASO with a bile salt alone (STC or GCA) or bile salt and a co-excipient improved permeability compared to unformulated ASO. Together, these results demonstrate bile salt formulations are effective for improving permeability of therapeutic oligonucleotides.

### Example 8: Correlation Analysis of In Vitro Permeability Studies and In Vivo Canine Bioavailability Studies

This Example describes a study that was performed to identify a dilution factor to be applied to the concentration of excipient(s) in a liquid formulation demonstrated to improve intestinal permeation (e.g., via GI-ORIS) in order to identify an amount of the same excipient(s) to include in a corresponding solid dosage form.

The study involved a correlation analysis between intestinal permeability as measured by GI-ORIS for an exemplary formulation and oral bioavailability in dogs for a solid dosage form containing the same active ingredient and excipient(s). Fourteen solid dosage formulations were generated containing vancomycin and one or more excipients. The solid dosage forms contained different types of excipients and different excipient amounts. The solid dosage formulations were formulated as immediate release tablets. The formulations were generated based on excipient composition used in the GI-ORIS screening experiments performed as described in the material and methods section The solid dosage formulations were orally administered to cohorts of beagles (n=2-5 per cohort). For absolute oral bioavailability calculation, dogs were also dosed IV with vancomycin at the same dose as the solid dosage forms. Blood samples were collected at 0, 0.33, 0.67, 1, 2, 4, 6, 7, 24, 48 hours following administration. The plasma was extracted from the whole blood samples and analyzed by LC-qTOF to quantify vancomycin concentration at the respective time point. Pharmacokinetic parameters were calculated by fitting data to a single compartment model.

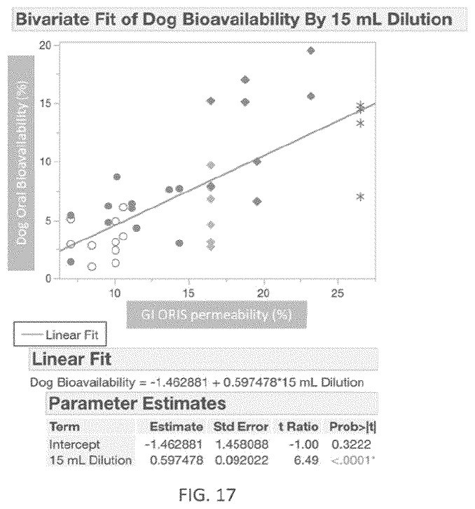

As shown in FIG. 17, a bivariate linear regression was applied to a plot of bioavailability for each solid dosage formulation versus intestinal permeability of its corresponding liquid dosage formulation as measured by GI-ORIS. To achieve a positive correlation, the empirical calculation yielded a dilution factor correction of 15 mL.

### Example 9: Pharmacokinetic Properties of a Vancomycin or Semaglutide Solid Dosage Form in Dogs

The pharmacokinetic properties of a solid dosage form containing vancomycin or semaglutide, a bile acid, and a co-excipient was evaluated in dogs following oral administration. Solid dosage formulations were prepared based upon excipient composition determined by the GI-ORIS screening.

The semaglutide solid dosage forms contained 14-28 mg of semaglutide and the excipients of solid dosage form 1 or 2 as listed in Table 19. The solid dosage forms were formulated for immediate release. The total amount of excipient and semaglutide in the solid dosage form was less than or about 1 g, which was considered a reasonable amount to include in tablet form and that would be tolerated following ingestion.

TABLE 19
Semaglutide Solid Dosage Forms

| Formulation Name | Excipient Amount |
|---|---|
| Semaglutide solid | 700 mg STC, 300 mg EDTA, and 40 mg PVP |
| dosage form 1 | |
| Semaglutide solid | 400 mg AAE, 550 mg STC, 50 mg C10, and |
| dosage form 2 | 100 mg EDTA |

Sodium caprate (C10); Sodium Taurocholate (STC); Sodium Ethylenedinitrilotetraacetate (EDTA); Polyvinyl pyrrolidone (PVP); Sodium Glutamate (AAE).

The vancomycin solid dosage forms contained 350 mg of vancomycin and the excipients of solid dosage forms 1, 2, 3, or 4 as listed in Table 20. The solid dosage forms were formulated for delayed release using an enteric coating or for immediate release by applying no coating, as indicated in the table.

TABLE 20
Vancomycin Solid Dosage Forms

| Formulation Name | Excipient Amount | Coating |
|---|---|---|
| Vancomycin solid | 350 mg GCA, 450 mg AAE, | Enteric |
| dosage form 1 | 70 mg EGTA, 20 mg MGS | coating |
| Vancomycin solid | 350 mg GCA, 450 mg AAE, | None |
| dosage form 2 | 70 mg EGTA, 20 mg MGS | |
| Vancomycin solid | 350 mg STC, 450 mg AAE, | Enteric |
| dosage form 3 | 70 mg EGTA, 20 mg MGS | coating |
| Vancomycin solid | 350 mg STC, 450 mg AAE, | None |
| dosage form 4 | 70 mg EGTA, 20 mg MGS | |

Sodium Taurocholate (STC);
Sodium Glutamate (AAE);
ethyleneglycol-bis(β-aminoethyl)-N,N,N′,N′-tetraacetate (EGTA);
magnesium stearate (MGS)

The semaglutide solid dosage forms were administered to beagles orally. Comparison was made to beagles administered Rybelsus, a commercially available oral semaglutide solid dosage form formulated for immediate release. Blood samples were collected prior to dosing and up to 48 hours following administration. The plasma was extracted from the whole blood samples and analyzed by LC-qTOF to quantify semaglutide concentration at the respective time point. Pharmacokinetic parameters (AUCtotal) were calculated by fitting data to a single compartment model.

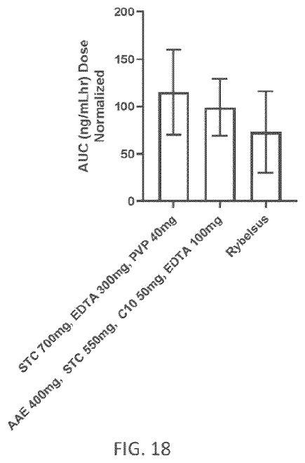

As shown in FIG. 18, the AUC of the dose-normalized semaglutide plasma concentration shows that the bile-acid containing solid dosage forms yielded comparable or higher exposure as compared to Ryblesus.

The vancomycin solid dosage forms were administered to beagles orally. For absolute oral bioavailability calculation, dogs were also dosed IV with vancomycin at the same dose as the solid dosage forms. Blood samples were collected prior to dosing and up to 48 hours following administration. The plasma was extracted from the whole blood samples and analyzed by LC-qTOF to quantify Vancomycin concentration at the respective time point. Bioavailability was calculated by fitting data to a single compartment model.

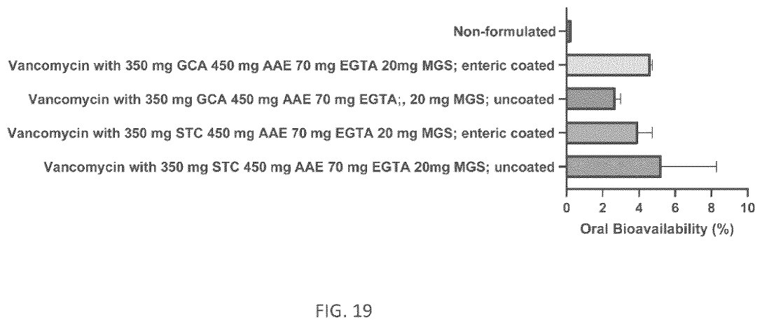

As shown in FIG. 19, the immediate release vancomycin solid dosage form containing STC and enteric coated vancomycin solid dosage form containing GCA yielded the highest level of bioavailability (5%). As comparison, the oral bioavailability of a non-formulated vancomycin solid dosage form (i.e., an oral enteric-coated capsule containing 100 mg vancomycin) orally administered to beagles is included (see Sauter, et al (2020) J. Pharm Sci 109:2090-2094). All solid dosage forms tested yielded bioavailability that was higher than the literature benchmark.

1. A composition comprising
(i) a compound selected from the group consisting of: (A) a polypeptide having a molecular weight of about 1 kDa to about 250 kDa, (B) a nucleic acid, and (C) a class III or class IV compound according to the Biopharmaceutical Classification System (BCS); and
(ii) an amount of at least one bile acid or salt thereof at an effective concentration greater than a critical micelle concentration (CMC) of the bile salt.

2. The composition of claim 1, wherein the amount of the at least one bile acid or salt thereof is effective to increase gastrointestinal (GI) permeability of the compound relative to GI permeability of the compound alone.

3. The composition of claim 1, wherein the polypeptide has a molecular weight greater than about 1 kDa, about 2 kDa, about 3 kDa, about 4 kDa, about 5 kDa, about 10 kDa, about 15 kDa, about 20 kDa, about 25 kDa, or about 30 kDa.

4. (canceled)

5. The composition of claim 1, wherein the polypeptide is an enzyme, an antibody or antigen binding fragment thereof, an antibacterial agent, a hormone, a growth factor, a chemokine, a cell signaling factor, or a cytokine.

6.-10. (canceled)

11. The composition of claim 1, wherein the nucleic acid comprises ribonucleosides, deoxyribonucleosides, or a combination thereof.

12. The composition of claim 1, wherein the nucleic acid is an immunostimulatory oligonucleotide, an mRNA, a plasmid DNA, or an RNA interference oligonucleotide.

13. The composition of claim 12, wherein the RNA interference oligonucleotide is an siRNA, a shRNA, a miRNA, or an antisense oligonucleotide.

14.-21. (canceled)

22. The composition of claim 1, wherein the class III or class IV compound has a molecular weight greater than about 1 kDa, about 2 kDa, about 3 kDa, about 4 kDa, about 5 kDa, about 10 kDa, about 15 kDa, about 20 kDa, about 25 kDa, or about 30 kDa.

23. (canceled)

24. The composition of claim 1, wherein the compound is a polypeptide, a nucleic acid, an oligosaccharide, a small molecule, a lipid, or a combination thereof.

25. The composition of claim 1, wherein the effective concentration of the at least one bile acid or salt thereof is at least about 3-fold, about 4-fold, about 5-fold, about 6-fold, about 7-fold, about 8-fold, about 9-fold, or about 10-fold greater than the CMC.

26. The composition of claim 1, wherein the effective concentration of the at least one bile acid or salt thereof is about 2-fold to about 200-fold greater than the CMC.

27. The composition of claim 1, wherein the effective concentration of the at least one bile acid or salt thereof is about 32 mg/mL to about 160 mg/mL.

28.-37. (canceled)

38. The composition of claim 1, wherein the at least one bile acid or salt thereof is selected from the group consisting of sodium taurocholate (STC), sodium glycocholate (GCA), sodium cholate (CHA), 3-[(3-Cholamidopropyl)dimethylammonio]-1-propanesulfonate (CHAPS), sodium taurodeoxycholate (TDCA), sodium glycodeoxycholate (GDCA), sodium deoxycholate (DCA), sodium taurochenodeoxycholate (TCDCA), sodium glycochenodeoxycholate (GCDCA), and sodium chenodeoxycholate (CDCA).

39. The composition of claim 1, wherein the bile acid or salt thereof has a CMC of less than about 30 mg/mL, about 20 mg/mL, about 15 mg/mL, or about 5 mg/mL.

40. (canceled)

41. The composition of claim 1, wherein the amount (mg) of the at least one bile acid or salt thereof is substantially equivalent to the effective concentration (mg/mL) multiplied by a dilution factor (mL) of about 2 mL to about 30 mL.

42-44. (canceled)

45. The composition of claim 1, wherein the amount of the at least one bile acid or salt thereof is about 60 mg to about 1,200 mg.

46.-109. (canceled)

110. A dosage form comprising a composition comprising
(i) a compound selected from the group consisting of: (A) a polypeptide having a molecular weight of about 1 kDa to about 250 kDa, (B) a nucleic acid, and (C) a class III or class IV compound according to the Biopharmaceutical Classification System (BCS); and
(ii) an amount of at least one bile acid or salt thereof at an effective concentration greater than a critical micelle concentration (CMC) of the bile salt.

111. The dosage form of claim 110, wherein the amount of the at least one bile acid or salt thereof is sufficient to achieve the effective concentration at a site of absorption in the GI tract.

112. The dosage form of claim 110, wherein the dosage form is a solid dosage form or a liquid dosage form.

113. (canceled)

114. The dosage form of claim 1_12, wherein the solid dosage form is formulated for immediate release or delayed release.

115.-124. (canceled)

125. A method of enhancing gastrointestinal (GI) permeability of a compound, the method comprising administering a composition comprising
(i) a compound selected from the group consisting of: (A) a polypeptide having a molecular weight of about 1 kDa to about 250 kDa, (B) a nucleic acid, and (C) a class III or class IV compound according to the Biopharmaceutical Classification System (BCS); and
(ii) an amount of at least one bile acid or salt thereof at an effective concentration greater than a critical micelle concentration (CMC) of the bile salt.

126-127. (canceled)

128. The method of claim 125, wherein the administering is enteral, rectal, intraduodenal, or oral.

129.-131. (canceled)

132. The method of claim 125, wherein the GI permeability of the compound is increased relative to GI permeability of a control composition.

133-134. (canceled)

135. The method of claim 125, wherein GI permeability is measured in vivo or in vitro using a transwell permeability assay, a Ussing chamber assay, or a GI Tract-Organ Robotic Interface System (GI-ORIS)-based permeability assay.

136. A method of increasing bioavailability of a compound in a subject, the method comprising administering the dosage form of claim 110 to the GI tract of the subject, wherein the dosage form comprises an effective dose of the compound.

137. The method of claim 136, wherein the bioavailability of the compound is increased relative to bioavailability of a control composition.

138.-150. (canceled)

151. A kit comprising a container comprising a composition or a dosage form comprising
(i) a compound selected from the group consisting of: (A) a polypeptide having a molecular weight of about 1 kDa to about 250 kDa, (B) a nucleic acid, and (C) a class III or class IV compound according to the Biopharmaceutical Classification System (BCS):
(ii) an amount of at least one bile acid or salt thereof at an effective concentration greater than a critical micelle concentration (CMC) of the bile salt; and
(iii) a package insert comprising instructions for administering the composition for increasing bioavailability of the compound in a subject from the GI tract.

152.-154. (canceled)
